SEQUENCE LISTING (24 sequences)

SEQ ID NO: 1 - DNA, 38 bp, Homo sapiens
  Coagulation factor V
gtaagagcagatccctggacaggcaaggaatacaggta

SEQ ID NO: 2 - DNA, 57 bp, Artificial Sequence
  Description of Artificial Sequence Synthetic first primer
  Description of Combined DNA/RNA Molecule Synthetic first primer
aactcagacaagatgtgatttttttacctgtatcucugaugcuuctacctgtattcc

SEQ ID NO: 3 - DNA, 70 bp, Artificial Sequence
  Description of Artificial Sequence Synthetic second primer
ctacagaactcagacaagatgtgaactacaatgttgctcatactacaatgtcacttactgtaagagcaga

SEQ ID NO: 4 - DNA, 56 bp, Artificial Sequence
  Description of Artificial Sequence Synthetic activator oligonucleotide
  Description of Combined DNA/RNA Molecule Synthetic activator oligonucleotide
uaaucuguaagagcagaucccuggacaggcaaggaauacaggtagaagcatcagag

SEQ ID NO: 5 - DNA, 52 bp, Artificial Sequence
  Description of Artificial Sequence Synthetic matrix oligonucleotide
aagctcgacaaaagaactcagagtgtgctcgacactacctgtattccttgcc

SEQ ID NO: 6 - DNA, 67 bp, Artificial Sequence
  Description of Artificial Sequence Synthetic activator oligonucleotide
gctcatactacaatgtcacttactgtaagagcagatccctggacaggcaaggaatacaggtaaaaaa

SEQ ID NO: 7 - DNA, 67 bp, Artificial Sequence
  Description of Artificial Sequence Synthetic matrix oligonucleotide
gctcatactacaatgtcacttactgtaagagcagatccctggacaggcgaggaatacaggtaaaaaa

SEQ ID NO: 8 - DNA, 70 bp, Artificial Sequence; identical to SEQ ID NO: 3
  Description of Artificial Sequence Synthetic second primer

SEQ ID NO: 9 - DNA, 67 bp, Artificial Sequence; identical to SEQ ID NO: 6
  Description of Artificial Sequence Synthetic matrix oligonucleotide

SEQ ID NO: 10 - DNA, 70 bp, Artificial Sequence; identical to SEQ ID NO: 3
  Description of Artificial Sequence Synthetic second primer

SEQ ID NO: 11 - DNA, 60 bp, Artificial Sequence
  Description of Artificial Sequence Synthetic mismatch activator oligonucleotide
  Description of Combined DNA/RNA Molecule Synthetic mismatch activator oligonucleotide
gctctaatctgtaagagcagatccctggacaggcaaagaauacaggtagaagcatcagag

SEQ ID NO: 12 - DNA, 70 bp, Artificial Sequence
  Description of Artificial Sequence Synthetic FVL variant with mutation
tgggctaataggactacttctaatctgtaagagcagatccctggacaggcaaggaatacaggtattttgt

SEQ ID NO: 13 - DNA, 70 bp, Artificial Sequence
  Description of Artificial Sequence Synthetic oligonucleotide
tgggctaataggactacttctaatctgtaagagcagatccctggacaggcgaggaatacaggtattttgt

SEQ ID NO: 14 - DNA, 59 bp, Artificial Sequence
  Description of Artificial Sequence Synthetic primer
  Description of Combined DNA/RNA Molecule Synthetic primer
cacaatcatcaatacttttttgtcaaaatacucugaugcucugtcaaaatacctgtatt

SEQ ID NO: 15 - DNA, 54 bp, Artificial Sequence
  Description of Artificial Sequence Synthetic primer
  Description of Combined DNA/RNA Molecule Synthetic primer
cacaatcatcaatacttttttgtcaaaatacucugaugcucugtcaaaatacct

SEQ ID NO: 16 - DNA, 28 bp, Artificial Sequence
  Description of Artificial Sequence Synthetic block oligonucleotide
  Description of Combined DNA/RNA Molecule Synthetic block oligonucleotide
cucugaugcucugucaaaatacctgaaa

SEQ ID NO: 17 - DNA, 76 bp, Artificial Sequence
  Description of Artificial Sequence Synthetic activator oligonucleotide
  Description of Combined DNA/RNA Molecule Synthetic activator oligonucleotide
aggacuacuucuaaucuguaagagcagaucccuggacaggcaaggaauacagguauuttgacagagcatcagagag

SEQ ID NO: 18 - DNA, 62 bp, Artificial Sequence
  Description of Artificial Sequence Synthetic second primer
cacaatcatcaatactaataggacctctgggctaataggactacttctaatctgtaagagca

SEQ ID NO: 19 - DNA, 36 bp, Artificial Sequence
  Description of Artificial Sequence Synthetic third primer
ctcgacactacttcaaggacaaaatacctgtattcc

SEQ ID NO: 20 - DNA, 41 bp, Artificial Sequence
  Description of Artificial Sequence Synthetic forth primer
ctctgggctaataggactacttctaatatgtaagagcagat

SEQ ID NO: 21 - DNA, 25 bp, Artificial Sequence
  Description of Artificial Sequence Synthetic third primer
acttcaaggacaaaatacctgtatt

SEQ ID NO: 22 - DNA, 19 bp, Artificial Sequence
  Description of Artificial Sequence Synthetic fourth primer
atatgtaagagcagatccc

SEQ ID NO: 23 - DNA, 15 bp, Artificial Sequence
  Description of Artificial Sequence Synthetic detection probe
ttgacaggcaaggaa

SEQ ID NO: 24 - DNA, 16 bp, Artificial Sequence
  Description of Artificial Sequence Synthetic probe
ttctggacaggcgagg

(19) United States
(12) Patent Application Publication
(10) Pub. No.: US 2021/0189479 A1
(43) Pub. Date: Jun. 24, 2021

(54) METHOD FOR SELECTIVE AMPLIFICATION OF NUCLEIC ACIDS AND KIT FOR PERFORMING THE SAME

(71) Applicant: AGCT GmbH, Luebeck (DE)

(72) Inventors: Christian Grunwald, Giessen (DE); Dmitry Cherkasov, Marburg (DE); Norbert BASLER, Großhansdorf (DE); Claus BECKER, Oetigheim (DE); Hans-Joerg HESS, Berlin (DE); Andreas MUELLER-HERMANN, Muenchen (DE)

(21) Appl. No.: 16/975,494

(22) PCT Filed: Feb. 25, 2019
(86) PCT No.: PCT/EP2019/054545

§ 371 Date: Aug. 25, 2020

(30) Foreign Application Priority Data
Feb. 26, 2018 (EP) 18158518.3

(51) Int. Cl. C12Q 1/6858

(52) U.S. Cl. CPC C12Q 1/6858

(57) ABSTRACT

A method is disclosed for detecting an amplification of nucleic acids in which use is essentially made of the fact that a predefined nucleic acid chain (target sequence) can be multiplied/amplified in the presence of a target-sequence-specific activator oligonucleotide. The target-sequence-specific activator oligonucleotide brings about the separation of de-novo synthesized complementary primer elongation products by means of strand displacement, so that a new primer oligonucleotide can attach to the respective strand of the template. The complex thus formed, of a primer oligonucleotide and a template strand, can initiate a new primer elongation reaction. The primer elongation products thus formed, in turn, act again as templates, the result being an exponentially proceeding amplification reaction. In the method, a selective amplification is effected in that, for the first, second primer and activator oligonucleotide, variants are used which differ from the target sequence by at least one nucleotide.

Today, synthesis of nucleic acid chains plays a central role in biotechnology. Methods like PCR have significantly developed both the research landscape and industrial fields of application such as diagnostics in medicine and food industry. The combination of PCR with other technologies such as sequencing, real-time detection, microarray technology, microfluidic management etc. has contributed to the technological development of the basic technology and was able to partly overcome some barriers of the PCR basic technology. Also, further amplification methods such as isothermal amplification techniques (LAMP, HDA, RPA, TMA, SDA etc.) have been developed. They have especially been intended for use in the field of POCT.

Despite enormous progress in this field PCR plays the central role and thus, defines the individual technological barriers of the applications.

One of the properties of common amplification methods such as PCR is that during the amplification operation of the nucleic acid the amplified sequence parts between both primers are not controlled. Substantially, primer binding is in focus of optimizations of PCR amplification reactions. At the beginning of the PCR amplification and its course continuously more or less specific primer binding and initiation of the synthesis of main products and by-products occur. For example, the by-products may be generated as a result of a non-specific primer extension event in a synthesis cycle. In case of a backward synthesis reaction that optionally takes place the non-specifically extended primer is read as a template what generally results in the formation of a complete primer binding site. Thus, an incorrect sequence information is transferred from one synthesis cycle to the next synthesis cycle what in the sum of synthesis cycles not only results in the initial generation, but above all in the exponential increase of by-products.

Such side reactions may possibly result in the initial generation and exponential increase of fragments that interfere with the main reaction (amplification of a target sequence) and result in interferences in following steps of analysis, respectively. Such by-products typically comprise primer sequences and corresponding primer sequences so that their amplification can be in parallel to the main reaction. Instead of a target sequence, however, such by-products comprise another nucleic acid sequence.

As a matter of priority, specificity of PCR amplification is achieved by optimizing the primer binding to target sequences. Here, for example additional oligonucleotides can be used that are capable of partially binding to primers and thus, competitively take part in the primer binding to other nucleic acid chains. Such probes generally bind to a sequence part of the primer and on the primer leave a single-stranded sequence part unoccupied, so that the primer with this part can bind to the target nucleic acid and initiate a synthesis reaction. Here, specificity of a primer binding is to be improved by the fact that primer template mismatches can competitively be displaced by such oligonucleotides. As a result, it is generally possible to improve the specificity of the initiation of PCR reactions. The effect of such oligonucleotides is limited to the interaction with primer sequences. Such additional oligonucleotides do not interact with the nucleic acid chain to be amplified in sections between both primers. However, due to a molar excess of primers, non-specific interactions of primers with templates can occur during amplification. If such a non-specific event of primer extension takes place (initiation of an exponential side reaction) a fully functional primer binding site is formed as a result or as part of backward synthesis of a complementary strand of the by-product. The presence of such a complete primer binding site in the by-product results in a loss of the competitive effect of such additional oligonucleotides on primer binding. Thus, controlling the specificity of the binding of a primer to the template by such oligonucleotides makes only initiation of a side reaction less likely, but can hardly affect its exponential amplification after a by-product has been generated.

Generally, the specificity of the synthesis of an amplification method can be improved with the reduced generation and co-amplification of by-products that differ from the target sequence, for example contribute to an improvement of diagnostic methods.

It is an object of the present invention to provide a method and components that enable enzymatic synthesis and amplification of nucleic acid chains. It is intended to provide a new enzymatic method and components for the synthesis of nucleic acid chains as well as the amplification of nucleic acid chains.

A further object of the invention is to provide a method with improved specificity of the synthesis of target nucleic acid chains in an exponential amplification.

In particular, single nucleotide differences are to be detected, as known for example for allele variants. Further, sequence differences between sequences having an improved specificity shall be detected that comprise several nucleotides, e.g., deletions/insertions.

It is a further object of the invention to provide methods that are able to simultaneously amplify several amplification fragments in one batch. In this way, multiplex amplification with improved specificity shall be enabled.

A further object of the invention is to provide means for implementing an exponential amplification method with improved specificity of the synthesis.

With the method according to the invention nucleic acid chains of a defined sequence composition are to be synthesized and amplified, respectively.

The problem of the invention is solved by providing amplification methods and corresponding means to perform them. Execution of the amplification method has already been described in PCT application (PCT/EP2017/071011) and the European application (EP-A 16185624.0). For details on performing the amplification the skilled person is referred to said application. The application is herewith entirely incorporated by reference.

Preferably, the amplification is an exponential amplification in which re-synthesized products of both primers (primer extension products) occur as templates for further synthesis steps. Here, primer sequences are at least partially copied, so that complementary primer binding sites are generated that are present as sequence segments of a double strand immediately after having been synthesized. In the amplification method synthesis steps of both strands and double strand opening steps of the re-synthesized sequence parts take place in mutual alternation. A sufficient double strand separation after a synthesis represents an important prerequisite for a further synthesis. Altogether, such an alternation of synthesis and double strand separation steps can result in an exponential amplification.

In the amplification method according to the invention the double strand opening of main products of the amplification (amplification of a target sequence-comprising nucleic acid chains) inter alia is by means of an oligonucleotide, referred to as activator oligonucleotide. The activator oligonucleotide preferably comprises sequence segments that correspond to the target sequence.

In detail, according to the invention strand separation is achieved by employing activator oligonucleotides having pre-defined sequences that preferably separate a re-synthesized double strand consisting of both specific primer extension products by means of a sequence-dependent nucleic acid mediated strand displacement. The resulting single-stranded segments of primer extension products comprise the target sequence as well as corresponding primer binding sites that can serve as binding sites for further primer oligonucleotides, so that an exponential amplification of nucleic acid chains to be amplified is achieved. Basically, the primer extension reactions and strand displacement reactions preferably take place at the same time. Amplification preferably takes place under reaction conditions that do not allow a spontaneous separation of both specific synthesized primer extension products.

Specific exponential amplification of a target sequence-comprising nucleic acid chain comprises a repetition of synthesis steps and double strand opening steps (activation steps for primer binding sites) as a mandatory prerequisite for the multiplication of the nucleic acid chain.

Opening of synthesized double strands is implemented as a reaction step that is to be sequence-specifically affected by the activator oligonucleotide. Said opening can be done completely, up to the dissociation of both complementary primer extension products, or may also be done partial.

According to the invention the activator oligonucleotide comprises sequence parts that can interact with the target sequence and further sequence parts that cause, permit or favor, respectively said interaction. In the course of the interaction with the activator oligonucleotide double-stranded sections of the synthesized primer extension products are converted into a single-stranded form via sequence-specific strand displacement. This process is sequence-dependent: only if the sequence of the synthesized double strand has a certain amount of complementarity with the corresponding sequence of the activator oligonucleotide a sufficient double strand opening occurs, so that the sequence parts essential for continuing the synthesis such as e.g. primer binding sites are converted into the single-stranded form, which corresponds to an “active state”. Thus, the activator oligonucleotide specifically “activates” the re-synthesized primer extension products comprising the target sequence for further synthesis steps.

In contrast, sequence parts that do not comprise a target sequence are not converted into the single-stranded state and remain as double strand, which corresponds to an “inactive” state. The potential primer binding sites in such a double strand are disadvantaged or completely prevented from interaction with new primers, so that further synthesis steps on such “non-activated” strands generally do not take place. This lacking or reduced activation (i.e. conversion into a single-stranded state) of synthesized nucleic acid strands after a synthesis step results in the fact that in the subsequent synthesis step only a reduced amount of primers can successfully take part in a primer extension reaction.

Due to an exponential amplification of main products (a nucleic acid chain to be amplified that comprises target sequences) to be aimed several synthesis steps and activation steps (double strand opening steps) are combined in one amplification method and performed or repeated, respectively until the desired amount of the specific nucleic acid chain is provided.

Here, the reaction conditions (e.g., temperature) are designed such that a spontaneous separation of complementary primer extension products in the absence of an activator oligonucleotide is unlikely or significantly decelerated.

Thus, the increase in the specificity of an amplification to be aimed results from the sequence-dependency of the separation of complementary primer extension products comprising a target sequence: the activator oligonucleotide enables or favors this double strand separation as a result of the matching of its sequence parts with given sequence parts of the primer extension products. This matching is verified after each synthesis cycle by the activator oligonucleotide. The exponential amplification results as a consequence from successful repetitions from synthesis processes and sequence-specific strand displacements by the activator oligonucleotide, i.e. “activations” (double strand openings/double strand separations/strand displacement processes resulting in a single-stranded form of corresponding primer binding sites) of re-synthesized primer extension products.

Mainly sequence-specific separation of the double strand assisted by an activator oligonucleotide may be combined in combination with sequence-specific or less sequence-specific primers.

The use of mainly specific activator oligonucleotides that are able to differ between individual variants for example makes it possible to arrange allele-specific or mutation-specific assays.

The invention is particularly suitable in the amplification of sequence variants of a known locus of a target sequence. Such a locus can comprise several occurring sequence variants of a target sequence and thus, is a polymorphous locus. Such a polymorphous locus for example comprises single nucleotide polymorphisms.

In order to achieve sequence specificity of a sequence variant of a locus of the target sequence components playing an essential role for the specificity of the amplification should be placed in a specific arrangement. Particularly advantageous are arrangements comprising a known sequence variant of a locus, wherein a mainly sequence-specific activator oligonucleotide as well as a corresponding primer can be designed.

Thus, the position of a sequence variant to be expected in the known locus is considered in designing of components in that at least the activator oligonucleotide, better activator oligonucleotide in combination with at least one primer, has a specific sequence composition in an amplification system that is suitable for amplification of a desired variant of the target sequence.

The problem of the invention further is solved in that an amplification system comprises at least one sequence-specific primer (e.g., allele-discriminating primer) in combination with one specific activator oligonucleotide (e.g., allele-specific activator oligonucleotide) each. Thus, for each specific composition of a target sequence and its specific variants (e.g., alleles) one specific combination of at least one specific primer and at least one specific activator oligonucleotide each can be provided. This results in a specific amplification of one of the possible sequence variants of a target sequence.

In addition, the problem of the invention is solved in that an amplification system comprises at least two, better four sequence-specific primers (e.g., allele-discriminating primers) in combination with one specific activator oligonucleotide each (e.g., allele-specific activator oligonucleotides). Thus, for each specific composition of a target sequence and its specific variants (e.g., alleles) one specific combination of at least one specific primer and at least one specific activator oligonucleotide each can be provided, wherein for all four nucleotide variants in a certain position a specific primer activator oligonucleotide is provided. The presence of at least one sequence variant of the target sequence in the reaction batch results in a specific amplification of one of the possible sequence variants of a target sequence.

In addition, the problem of the invention is solved in that an amplification system comprises at least one sequence-specific primer (e.g., allele-discriminating primer) in combination with one specific activator oligonucleotide each (e.g., allele-specific activator oligonucleotides) and at least one further primer (a competitor primer) which has a sequence composition complementary to other sequence variants to be expected. Thus, for each specific composition of a target sequence and its specific variants (e.g., alleles) one specific combination of at least one specific primer and at least one specific activator oligonucleotide can be provided each. This results in a specific amplification of one of the possible sequence variants of a target sequence.

The problem of the invention is further solved in that both strands of a double-stranded nucleic acid chain are analyzed, wherein in a reaction there are provided the components of an amplification system for one strand of a target nucleic acid that is used as a start nucleic acid, and in a separate batch there are provided components for the complementary strand of the target nucleic acid chain that is used as the start nucleic acid. Thus, at least two activator oligonucleotides are used that each can bind to a strand of the target nucleic acid chain (as a first primer extension product). Both activator oligonucleotides are used in separate reaction batches.

In addition, the problem of the invention is solved in that an amplification system comprises at least one primer that is able to support the amplification of several potential sequence variants (e.g., a target sequence-specific primer, but no allele-discriminating primer, a target sequence-specific, but an allele-unspecific primer) in combination with one specific activator oligonucleotide each (e.g., allele-specific activator oligonucleotides). Thus, for each specific composition of a target sequence and its specific variants (e.g., alleles) one specific combination of at least one allele-unspecific primer and at least one allele-specific activator oligonucleotide each can be provided. This results in a specific amplification of one of the possible sequence variants of a target sequence.

In addition, the problem of the invention is solved in that at least one primer that is able to potentially support the amplification of several sequence variants (e.g., a target sequence-specific, but not allele-discriminating primer, allele-unspecific primer) is used in combination with one activator oligonucleotide, wherein the activator oligonucleotide is able to amplify at least two sequence variants of a target sequence. Thus, in the presence of at least one of the variants as a start nucleic acid chain in the reaction one of these two variants can be amplified what is assisted by only one activator oligonucleotide. Thus, such an activator oligonucleotide is able to support amplification of at least two sequence variants. In this way, amplification can also take place if a target sequence is present in the reaction batch, but it is not known exactly which exact composition a polymorphous locus does comprise.

The oligonucleotides required for an amplification (at least one target sequence-specific first primer, at least one target sequence-specific second primer, at least one target sequence-specific activator oligonucleotide) are combined as target sequence-specific amplification system.

Since also several target sequences may be present in the batch by use of several target sequence-specific amplification systems in one reaction batch parallel amplification of more than one target sequence should be made possible, wherein preferably target sequence-specific components are combined each.

Thus, using a pre-defined activator oligonucleotide enables a sequence-dependent verification of the contents of primer extension products between individual synthesis steps during the exponential amplification and obtaining a selection or choice of sequences for subsequent synthesis steps. Here, distinction can be made between “active”, single-stranded states of re-synthesized specific primer extension products as a result of a successful interaction with an activator oligonucleotide, and “inactive”, double-stranded states of re-synthesized non-specific primer extension products as a result of a deficient and/or insufficient and/or reduced and/or decelerated interaction with an activator oligonucleotide.

The following effects result for an exponential amplification:

Under non-denaturant conditions separation of specifically synthesized strands takes place with cooperation of an activator oligonucleotide.

Exponential amplification of target sequence-comprising nucleic acid chains is sequence-controlled (main reaction). Said sequence control takes place after each synthesis step and includes sequence segments lying between primers and comprising a target sequence. The successful verification of the results of the synthesis after each synthesis step results in the separation of both specific primer extension products, which is the prerequisite for further specific synthesis steps.

During the amplification an initial generation of non-specific primer extension products basically cannot be excluded (by-products). Due to a template dependency such non-specific primer extension products immediately after their synthesis are generally present in the double-stranded form. However, the interaction with the activator oligonucleotide either completely fails to come or is limited, so that there is no strand separation or the strand separation is decelerated over the main reaction. Thus, there is no transfer of incorrect sequence information from one synthesis cycle to the next.

By the choice of the reaction conditions and the design of an activator oligonucleotide it is thus possible to specifically affect the efficacy of the regeneration of correct nucleic acid chain templates between single synthesis steps during an amplification method. Generally, the higher the degree of matching of the synthesized sequence with the given sequence of the activator oligonucleotide, the more successful the separation of the synthesized products and in turn the more successful the regeneration of correct templates from one synthesis step to the next. On the other hand, a sequence divergence in by-products results in an insufficient regeneration of template strands and thus, in a deceleration of the synthesis initiation and a reduction of the yield in each subsequent cycle. The whole exponential amplification of by-products either proceeds more slowly or does not takes place at all and/or remains at an undetectable level.

Thus, the method enables a verification of the synthesized sequences in real-time, i.e. without stopping the reactions and thus, represents potential for the development of homogeneous assays in which all components of the assay are already present in the reaction mixture at the beginning of a reaction.

### Terms and Definitions

In the context of the present invention the terms used have the following meaning:

The term “oligonucleotide”, as used here with respect to primers, activator oligonucleotide, probes, nucleic acid chain to be amplified, is defined as a molecule comprising two or more, preferably more than three deoxyribonucleotides and/or ribonucleotides and/or nucleotide modifications and/or non-nucleotide modifications. Its length comprises for example regions between 3 to 300 nucleotide units or analogues thereof, preferably between 5 to 200 nucleotide units or analogues thereof. Its exact size depends on a number of factors that in turn depend on the final function or use of the oligonucleotides.

The term “primer”, as used herein, relates to an oligonucleotide, regardless of whether it is naturally occurring, e.g. in a purified restriction cleavage, or was synthetically produced. A primer is capable of acting as an initiation point of the synthesis if it is used under conditions in which the synthesis of a primer extension product that is complementary to a nucleic acid strand is induced, i.e. in the presence of nucleotides and an inducing agent such as e.g., DNA polymerase at a suitable temperature and a suitable pH value. Preferably, the primer for a maximum efficacy in the amplification is single-stranded. The primer has to be sufficiently long in order to initiate synthesis of the extension product in the presence of the inducing agent. The exact length of the primer depends on a number of factors, including the reaction temperature and the primer source and the application of the method. For example, the length of the oligonucleotide primer in diagnostic applications, according to the complexity of the target sequence, is between 5 to 100 nucleotides, preferably 6 to 40, and especially preferred 7 to 30 nucleotides. Short primer molecules generally require lower reaction temperatures to carry out their primer function in order to form sufficiently stable complexes with the template, or higher concentrations of other reaction components, for example DNA polymerases, so that primer template complexes formed can sufficiently be lengthened.

The primers used here are selected such that they are “substantially” complementary to the various strands of each specific sequence to be amplified. This means, that the primers have to be sufficiently complementary to hybridize with their respective strands and to initiate a primer extension reaction. Thus, for example the primer sequence does not have to reflect the exact sequence of the target sequence. For example, a non-complementary nucleotide fragment can be attached to the 5′ end of the primer, wherein the remaining primer sequence is complementary to the strand. In another embodiment single non-complementary bases or longer non-complementary sequences can be inserted into a primer, provided that the primer sequence has a sufficiently large complementarity with the sequence of the strand to be amplified, in order to hybridize therewith and thus, generate a primer template complex capable for the synthesis of the extension product.

In the course of the enzymatic synthesis of a strand complementary to the template a primer extension product is generated that is completely complementary to the template strand.

Allele-specific primers are primers that due to their sequence composition preferably under stringent reaction conditions are able to hybridize to respective individual sequence variants of a target sequence and can be extended by a polymerase using the target sequence. Individual allele-specific primers may be combined in one group that covers all the variants of a common target sequence. Such a group of allele-specific primers comprises at least two different allele-specific primers, since a polymorphous locus in a given position in the target sequence comprises at least two sequence variants. The allele-specific primers are designed such that under stringent reaction conditions they preferably bind to their respective specific perfect-match template and thus, use this specific perfect-match template to form the respective primer extension products under the catalytic action of the polymerase. Preferably, 3′-terminal segments of allele-specific primers may be used to discriminate variants of target sequences and as a result may be adapted in their sequence composition to the respective variants such that such primers form a perfect-match double strand with the respective variant under stringent conditions. Generally, such perfect-match double strands may be well recognized by a polymerase and under suitable reaction conditions primer extension takes place. Thus, if an allele-specific primer interacts with another variant of a target sequence a mismatch double strand is formed. Generally, such mismatches result in a delay of the extension by a polymerase or in a deceleration of the entire reaction. In one embodiment allele-specific primers in the 3′ segment can comprise at least one phosphorothioate bond which protects allele-specific primers against 3′-5′ nuclease decomposition by a polymerase.

Thus, several allele-specific primers comprise sequence segments which for one group of allele-specific primers are substantially identic or uniform, respectively as well as sequence segments which in the primers of one group are different and characteristic for the respective sequence variant of a target sequence. By including uniform sequence segments such primers are able to hybridize to the respective target sequence under reaction conditions. By including characteristic sequence segments a respective primer can specifically bind to a sequence variant of the target sequence to form a perfect-match double strand. Preferably, the primers are constructed such that under the reaction conditions used binding to a target sequence to form a perfect-match double strand is preferred and binding to a target sequence to form a mismatch double strand is less preferred.

In one embodiment the first primer oligonucleotide is provided as an allele-specific primer in combination with a respective allele-specific activator oligonucleotide. In a further embodiment the second primer oligonucleotide is provided as an allele-specific primer in combination with a respective allele-specific activator oligonucleotide.

Tm—Melting Temperature

The melting temperature of a complementary or partially complementary double strand is generally understood to be a measured value of a reaction temperature at which ca. half of the strands is present as a double strand and the other half is present as a single strand. The system (association and dissociation of strands) is in equilibrium.

Due to a number of factors that can affect the Tm of a double strand (e.g., sequence length, CG content of the sequence, buffer conditions, concentration of divalent metal cations, etc.) the Tm of a nucleic acid to be amplified is to be determined under the same conditions as the intended amplification reaction.

Because the measurable melting temperature depends on multiple reaction parameters, e.g., the respective buffer conditions and respective concentrations of the reaction partners, the melting temperature is meant to be a value that was measured in the same reaction buffer as the exponential amplification, at concentrations of both complementary components of a double strand of about 0.1 μmol/l to about 10 μmol/l, preferably in a concentration of about 0.3 μmol/l to ca. 3 μmol/l, preferably at ca. 1 μmol/l. The respective value of the melting temperature is a guide value that correlates with the stability of a corresponding double strand.

A melting temperature for a double strand can be roughly estimated. Several commercial suppliers enable a theoretic calculation of a melting temperature to be expected. For example, software package OligoAnalyzer 3.1 (on-line accessible at IDT (Integrated DNA-Technologies)) can be used to estimate the strength of the bonds of individual oligonucleotides.

The deoxyribonucleoside triphosphates (dNTPs) dATP, dCTP, dGTP, and TTP (or dUTP, or dUTP/TTP mixture) are added to the synthesis mixture in adequate amounts. In one embodiment at least one further type of dNTP analogues can be added to the synthesis mixture in addition to the dNTPs. In one embodiment, these dNTP analogues comprise for example a characteristic mark (e.g., biotin or fluorescent dye), so that when built into a nucleic acid strand also this mark is integrated in the nucleic acid strand. In another embodiment, these dNTP analogues comprise at least one modification of the sugar phosphate proportion of the nucleotide, e.g., alpha-phosphorothioate-2′-deoxyribonucleoside triphosphates (or other modifications imparting a nuclease resistance to a nucleic acid strand), 2′,3′-dideoxy-ribonucleoside triphosphates, acyclo-nucleoside triphosphates (or other modifications resulting in the termination of a synthesis). In a further embodiment, these dNTP analogues comprise at least one modification of a nucleobase, e.g., iso-cytosines, iso-guanosines (or also other modifications of the nucleobases of the extended genetic alphabet), 2-amino-adenosines, 2-thiouridines, inosines, 7-deaza-adenosines, 7-deaza-guanosines, 5-me-cytosines, 5-propyl-uridines, 5-propyl-cytosines (or also other modifications of nucleobases that can be built in by a polymerase compared to natural nucleobases and result in the change of the strand stability). In a further embodiment, a dNTP analogue comprises both a modification of the nucleobase and a modification of the sugar phosphate proportion. In a further embodiment, at least one further type of dNTP analogues is added to the synthesis mixture instead of the at least one natural dNTP substrate.

The agent inducing the nucleic acid synthesis can be an enzyme-entrapping compound or a system that acts such that as a result the synthesis of the primer extension product is caused. Suitable enzymes for this purpose comprise e.g., DNA polymerases such as Bst polymerase and its modifications, Vent polymerase and other—preferably thermo-stable DNA polymerases that enable the incorporation of the nucleotides in the correct manner, whereby the primer extension products are formed that are complementary to each synthesized nucleic acid strand. Generally, the synthesis is initiated on the 3′ end of each primer and then progresses toward the 5′ direction along the template strand until the synthesis is completed or interrupted.

Preferably, there are used polymerases that are capable of strand displacement. These include for example the large fragment of the Bst polymerase or its modifications (e.g., Bst 2.0 DNA polymerase), the Klenow fragment, Vent exo minus polymerase, Deepvent exo minus DNA polymerase, a large fragment of the Bsu DNA polymerase, a large fragment of the Bsm DNA polymerase.

In one embodiment, there are preferably employed polymerases that have no 5′-3′-exo-nuclease activity or no 5′-3′-FEN activity, respectively.

In one embodiment, at least two different polymerases are employed, for example polymerases capable of strand displacement and such that have a 3′-5′-proof reading activity.

In preferred embodiments, there are employed polymerases with a hot start function that only exert their function after having reached a certain temperature.

First Primer Oligonucleotide:

The first primer oligonucleotide comprises a first primer region and a second region. The first primer region is able to bind to a substantially complementary sequence within the nucleic acid to be amplified or equivalents thereof and to initiate a primer extension reaction. The second region comprises a polynucleotide tail that is able to bind to an activator oligonucleotide and thus, to cause a spatial proximity between the activator oligonucleotide and other parts of the first primer extension product that is sufficient to initiate a strand displacement by the activator oligonucleotide. The second region of the first primer oligonucleotide further comprises at least one modification (a nucleotide modification or non-nucleotide modification) that prevents the polymerase from copying the polynucleotide tail by inhibiting the continuation of the polymerase-dependent synthesis. Said modification is located for example at the transition between the first and the second regions of the first primer oligonucleotide. Accordingly, the first primer region of the first primer oligonucleotide can be copied by a polymerase, so that a sequence complementary to this region can be generated by the polymerase during the synthesis of the second primer extension product. The polynucleotide tail of the second region of the first primer oligonucleotide is preferably not copied by the polymerase. In one embodiment, this is achieved by the modification in the second region that stops the polymerase before the polynucleotide tail. In a further embodiment, this is achieved by nucleotide modifications in the second region, wherein the entire polynucleotide tail substantially consists of such nucleotide modifications and thus, cannot be copied by polymerase.

In one embodiment, each first primer oligonucleotide is specific for one nucleic acid to be amplified each.

In one embodiment, each first primer oligonucleotide is specific for at least two of the nucleic acids to be amplified that each comprise substantially different sequences.

In one embodiment, the first primer oligonucleotide is labeled with a characteristic marker, e.g., a fluorescent dye (e.g., TAMRA, fluorescein, Cy3, Cy5) or an affinity marker (e.g., biotin, digoxigenin) or an additional sequence fragment, e.g., for binding a specific oligonucleotide probe for detection or immobilization or barcode labeling.

In one embodiment at least one first primer oligonucleotide is provided as an allele-specific primer in combination with at least one corresponding allele-specific activator oligonucleotide.

Second Primer Oligonucleotide:

Oligonucleotide that with its 3′ segment is able to bind to a substantially complementary sequence within the nucleic acid to be amplified or equivalents thereof and to initiate a specific second primer extension reaction. Thus, this second primer oligonucleotide is able to bind to the 3′ segment of a first specific primer extension product of the first primer oligonucleotide and to initiate a polymerase-dependent synthesis of a second primer extension product.

The length of the second primer oligonucleotide can be between 15 and 100 nucleotides, preferably between 20 and 60 nucleotides, particularly preferred between 30 and 50 nucleotides.

In one embodiment, each of the second primer oligonucleotides is specific for one nucleic acid to be amplified each.

In one embodiment, each of the second primer oligonucleotides is specific for at least two of the nucleic acids to be amplified that each comprise substantially different sequences.

In one embodiment, the second primer oligonucleotide is labeled with a characteristic marker, e.g., a fluorescent dye (e.g., TAMRA, fluorescein, Cy3, Cy5) or an affinity marker (e.g., biotin, digoxigenin) or an additional sequence fragment, e.g., for binding a specific oligonucleotide probe for detection or immobilization or barcode labeling.

In one embodiment at least one second primer oligonucleotide is provided as an allele-specific primer in combination with at least one corresponding allele-specific activator oligonucleotide.

Primer Extension Product:

A primer extension product (also referred to as primer elongation product) is generated by enzymatic (polymerase-dependent) extension of a primer oligonucleotide as a result of a template-dependent synthesis that is catalyzed by a polymerase.

A primer extension product comprises the sequence of the primer oligonucleotide in its 5′ segment and the sequence of the extension product (also referred to as elongation product) that was synthesized by a polymerase in a template-dependent manner. The extension product synthesized by the polymerase is complementary to the template strand to which it was synthesized.

A specific primer extension product (main product) comprises sequences of the nucleic acid chain to be amplified. It is the result of a specific synthesis or a proper performance of an intended primer extension reaction in which the nucleic acid chain specifically to be amplified serves as a template. In a preferred embodiment, the sequence of the synthesized primer extension products completely corresponds to the expected sequence of a nucleic acid to be amplified. In another embodiment, divergences in the obtained sequence from the theoretically expected sequence can be tolerated. In one embodiment, the degree of matching of the sequence obtained as a result of an amplification with the sequence of the theoretically expected nucleic acid to be amplified is for example between 90% and 100%, preferably the matching is above 95%, ideally the matching is above 98% (based on the proportion of the synthesized bases).

The length of the extension product of a specific primer extension product can be between 10 and 300 nucleotides, better between 10 and 180 nucleotides, preferably between 20 and 120 nucleotides, particularly preferably between 30 and 80 nucleotides.

A non-specific primer extension product (by-product) comprises for example sequences that have been generated as a result of a non-specific or incorrect or unintended primer extension reaction. These include for example primer extension products that have been generated as a result of a false initiation result (false priming) or as a result of other side reactions, including polymerase-dependent sequence changes such as base substitution, deletion etc. The degree of sequence divergences of non-specific primer extension products generally exceeds the ability of activator oligonucleotides to successfully displace such double-stranded by-products from their templates, so that amplification of such by-products proceeds slower or is completely absent. The degree of acceptance or the limit of tolerance for divergences for example depends on reaction temperatures and the type of sequence divergence. Examples of non-specific primer extension products are primer dimers or sequence variants that do not correspond to the nucleic acid to be amplified, e.g., sequences that do not comprise a target sequence.

Assessment as to a sufficient specificity of the amplification is often linked to the problem formulation. In many amplification methods a certain degree of non-specificity of the amplification reaction can be tolerated as long as the desired result can be obtained. In a preferred embodiment, the proportion of nucleic acid chains to be amplified in the total result of the reaction is more than 1%, better more than 10%, more preferably more than 30%, based on the total amount of re-synthesized strands.

Nucleic Acid to be Amplified

The nucleic acid to be amplified is a nucleic acid chain that is to be sequence-specifically or at least mainly sequence-specifically amplified by the polymerase by means of the exponential amplification by employing primers and activator oligonucleotides.

The length of the nucleic acid to be amplified can be between 20 and 300 nucleotides, better between 30 and 200 nucleotides, preferably between 40 and 150 nucleotides, particularly preferred between 50 and 100 nucleotides.

The nucleic acid chain to be amplified can comprise one or more target sequences or equivalents thereof. Furthermore, a nucleic acid to be amplified can comprise the sequences that are substantially complementary to a target sequence and that are multiplied with a similar efficacy such as a target sequence in an amplification reaction and comprises a target sequence or sections thereof. In addition to a target sequence the nucleic acid to be amplified can further include sequence segments, for example primer sequences, sequences with primer binding sites and/or sequence segments for binding detection probes, and/or sequence segments for sequence coding of strands by barcode sequences and/or sequence segments for binding to a solid phase. The primer sequences or sequence portions thereof as well as primer binding sites or sequence portions thereof may for example belong to sequence parts of a target sequence.

In one embodiment, the nucleic acid to be amplified corresponds to the target sequence.

In another embodiment, the target sequence forms a part of the sequence of the nucleic acid chain to be amplified. Such a target sequence can be flanked by the 3′ side and/or 5′ side of further sequences. These further sequences can for example comprise binding sites for primers or portions thereof, and/or primer sequences or portions thereof, and/or binding sites for detection probes, and/or adaptor sequences for complementary binding to a solid phase (e.g., in the context of microarrays, or bead-based analyses) and/or barcoding sequences for a digital signature of sequences.

To start the amplification a nucleic acid chain has to be added to the reaction mixture at the beginning of the reaction that acts as the initial template for the synthesis of the nucleic acid chain to be amplified. Said nucleic acid chain is referred to as the start nucleic acid chain. Said start nucleic acid chain prescribes the arrangement of individual sequence elements that are relevant for the formation/synthesis/exponential amplification of a nucleic acid chain to be amplified.

In a preferred embodiment, the initial template (start nucleic acid chain), that is added to an amplification reaction at the beginning or is added to the reaction mixture, corresponds to the sequence composition of the nucleic acid chain to be amplified.

In initial stages of the amplification reaction and in its further course the respective primers bind to the corresponding binding sites in the start nucleic acid chain and initiate the synthesis of specific primer extension products. Such specific primer extension products during the amplification exponentially accumulate and increasingly take the role of templates for the synthesis of complementary primer extension products in an exponential amplification.

By the repeated template-dependent synthesis processes during an exponential amplification there is formed thus the nucleic acid chain to be amplified.

Toward the end of an amplification reaction the main product of the reaction (the nucleic acid to be amplified) can mainly be single-stranded or mainly form a complementary double strand. This can for example be determined by the relative concentrations of both primers and the appropriate reaction conditions.

Equivalents of the nucleic acid to be amplified comprise nucleic acids of substantially identical information content. For example, complementary strands of a nucleic acid to be amplified have an identical information content and may be referred to as being equivalent.

Target Sequence

In one embodiment, a target sequence is a segment of a nucleic acid chain to be amplified that can serve as the characteristic sequence of the nucleic acid to be amplified. Said target sequence can serve as a marker for the presence or absence of another nucleic acid. Thus, said other nucleic acid serves as a source of the target sequence and for example can comprise a genomic DNA or RNA or parts of the genomic DNA or RNA (e.g., mRNA), or equivalents of the genomic DNA or RNA of an organism (e.g., cDNA, modified RNA such as rRNA, tRNA, microRNA etc.), or defined changes of the genomic DNA or RNA of an organism, for example mutations (e.g., deletions, insertions, substitutions, additions, sequence multiplication, e.g., repeat multiplication in context of microsatellite instability), splice variants, rearrangement variants (e.g., T cell receptor variants) etc. The individual target sequences may stand for a phenotypic feature, for example for antibiotic resistance or have prognostic information and thus, be of interest for diagnostic assays/tests. As the source/origin for a target sequence such a nucleic acid can for example comprise the target sequence as a sequence element of its strand. Thus, a target sequence can serve as a characteristic marker for a certain sequence content of another nucleic acid.

The target sequence can be single-stranded or double-stranded. It can be substantially identical to the nucleic acid to be amplified or only represent a part of the nucleic acid to be amplified.

Equivalents of the target sequence comprise nucleic acids of substantially identical information content. For example, complementary strands of a target sequence have an identical information content and can be referred to as being equivalent. Also, RNA and DNA variants of a sequence are examples of an equivalent information content.

In context of the material preparation for an amplification reaction such a target sequence can be isolated from its original sequence environment and prepared for the amplification reaction.

In a preferred embodiment, a nucleic acid to be amplified comprises a target sequence. In one embodiment, the target sequence corresponds to the nucleic acid to be amplified. In a further preferred embodiment, a start nucleic acid chain comprises a target sequence. In one embodiment, the target sequence corresponds to a start nucleic acid chain.

Allele Variants of a Target Sequence/Polymorphous Locus of a Target Sequence

A target sequence (e.g., a characteristic segment of a DNA or RNA) can comprise several naturally occurring sequence variants or artificially inserted sequence alterations. Such variants are often referred to as polymorphisms of a locus or allele sequences. In addition, said variants of a target sequence can depict specific alterations of the natural state of the DNA that are formed e.g. by mutations/mutagenesis and in propagation of a cell mass represent characteristic features of such a clonal propagation process (e.g., with cancer diseases or in biotechnologically utilizable stems). Individual allele sequences of a target sequence may be sequence differences that comprise single nucleotide positions (e.g., A->G or T->C substitutions or single nucleotide insertions or deletions, respectively) or a sequence difference can also comprise several nucleotides (e.g., 2 to 200 nucleotides). These may be for example deletions/insertions/transversions/duplications etc. A sequence segment comprising such sequence variants of a target sequence is often referred to as a polymorphous locus. A target sequence can comprise one or more polymorphous loci.

Generally, the sequence positions of individual allele variants of a target sequence are known and thus, allow a design of sequence-specific reaction components, e.g., activator oligonucleotides or of combinations comprising an activator oligonucleotide and a first primer or an activator oligonucleotide and a second primer.

Embodiments of Target Sequences:

In one embodiment a target sequence that can comprise several sequence variants in one polymorphous locus further comprises at least one first target sequence segment that is characteristic and uniform for all the target sequence variants of a target sequence (target sequence group). In a further embodiment a target sequence comprises at least two target sequence segments (a first target sequence segment and a second target sequence segment), wherein each of said target sequence segments is characteristic and uniform for all the target sequence variants of a target sequence (target sequence group). Such uniform target sequence segments are preferably located on both sides of a polymorphous locus and thus, flank a polymorphous locus (with sequence variants) of a target sequence from both sides. Preferably, the first uniform target sequence segment in its sequence composition differs from the sequence composition of the second uniform target sequence segment in at least one nucleotide position.

The length of a polymorphous locus of a target sequence can comprise ranges from one nucleotide up to 200 nucleotides, better from one nucleotide up to 100 nucleotides, preferably from one nucleotide up to 50 nucleotides, particularly preferred from one nucleotide up to 20 nucleotides. The lengths of a uniform sequence segment (first and second uniform sequence segments) can comprise the following ranges at least for one of the two segments: from 6 to 300 nucleotides, better from 10 to 200 nucleotides, preferably from 15 to 100 nucleotides, particularly preferred from 20 to 100 nucleotides.

In one embodiment individual sequence variants of a target sequence group can be combined, wherein such variants define at least one sequence segment that is uniform and specific for said target sequence (target sequence group). Thus, individual sequence variants of such a target sequence (target sequence group) differ by sequence segments of the polymorphous locus.

In a further embodiment, individual sequence variants of a target sequence group can be combined, wherein such variants define at least two sequence segments that are uniform and specific for said target sequence (target sequence group). Thus, individual sequence variants of such a target sequence (target sequence group) differ by sequence segments of the polymorphous locus. Preferably, the polymorphous locus is between both uniform sequence segments.

In one embodiment two different target sequences each may specifically be amplified in one amplification reaction.

Different target sequences are characterized in that they preferably can be differentiated among each other by sequences that are specific and characteristic for each target sequence. Preferably, different target sequences can be differentiated in their total length each by specific and characteristic sequences and thus, do not comprise any sequence segments that are identical for both target sequences. In a further embodiment different target sequences can comprise at least one sequence segment that is substantially similar or even identical for at least two different target sequences. The length of such a sequence segment is equal to or less than 19 nucleotides (counted as nucleotide positions coupled to each other), better equal to or less than 14 nucleotides (as nucleotide positions coupled to each other), preferably equal to or less than 9 nucleotides (as nucleotide positions coupled to each other), particularly preferred equal to or less than 5 nucleotides (as nucleotide positions coupled to each other).

Start Nucleic Acid Chain

To start the amplification a nucleic acid chain has to be added to the reaction mixture at the beginning of the reaction that acts as the initial template for the synthesis of the nucleic acid chain to be amplified. Said nucleic acid chain is referred to as the start nucleic acid chain. Said start nucleic acid chain prescribes the arrangement of individual sequence elements that are relevant for the formation/synthesis/exponential amplification of a nucleic acid chain to be amplified.

Such a start nucleic acid chain can be single-stranded or double-stranded at the beginning of the reaction. If the complementary strands of the start nucleic acid chain are separated from each other the strands, regardless of whether the nucleic acid originally was double- or single-stranded, can serve as a template for the synthesis of specific complementary primer extension products.

The start nucleic acid chain in one embodiment comprises a target sequence or its subsegments that comprise at least one sequence variant of a polymorphous locus of the target sequence to be expected. A start nucleic acid chain in a further embodiment comprises a target sequence or its subsegments that comprise at least one sequence variant of a polymorphous locus of the target sequence to be expected and at least one uniform sequence segment of a target sequence.

A start nucleic acid chain in a further embodiment comprises a target sequence or its subsegments that comprise at least one sequence variant of a polymorphous locus of the target sequence to be expected and at least one uniform sequence segment of a target sequence, wherein uniform sequence segments of a target sequence on both sides flank the polymorphous locus with possible sequence variants.

Further, a start nucleic acid comprises at least one mainly single-stranded sequence segment to which at least one of the primers of the amplification system can mainly complementary bind with its 3′ segment, so that the polymerase used can extend such a primer, when hybridized to the start nucleic acid chain, template-specific by incorporating dNTPs.

Activator Oligonucleotide:

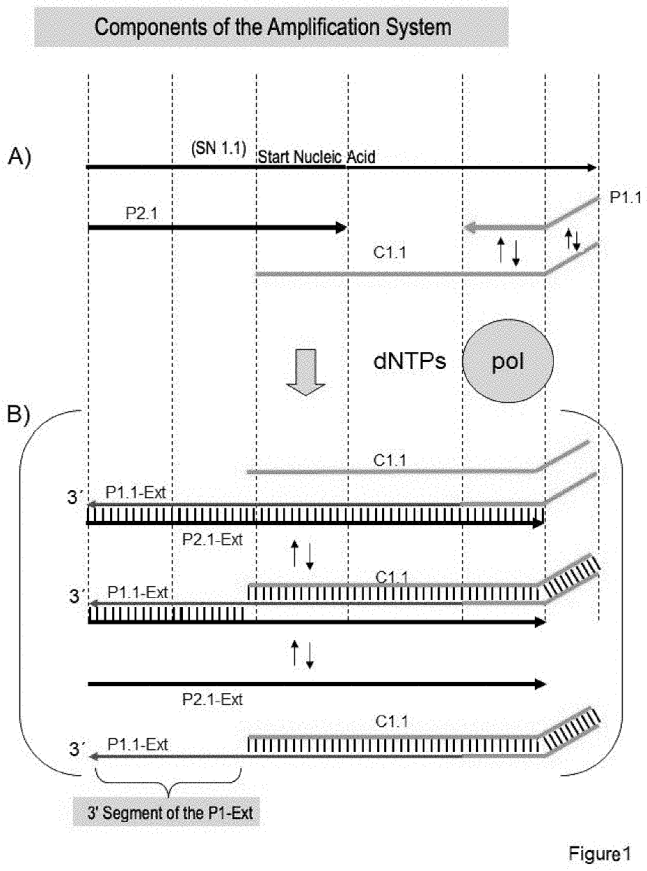

The activator oligonucleotide (FIG. 1) is a preferably single-stranded nucleic acid chain that includes a pre-defined substantially complementary sequence in a part of the first primer extension product that is specifically generated during the amplification of the nucleic acid to be amplified. In this way, the activator oligonucleotide can substantially complementary bind to the first primer oligonucleotide and at least to the 5′ segment of the specific extension product of the first primer oligonucleotide. In one embodiment, the activator oligonucleotide in its inner sequence segment comprises nucleotide modifications that prevent polymerase from synthesizing a complementary strand using the activator oligonucleotide as a template if the first primer oligonucleotide is complementary bound to the activator oligonucleotide. The activator oligonucleotide under the chosen reaction conditions is further able to completely or partially displace the second specific primer extension product from the binding with the first specific primer extension product via strand displacement. Here, the activator oligonucleotide with its complementary regions is attached to the first specific primer extension product. In case of a successful binding between the activator oligonucleotide and the first specific primer extension product this results in a restoration of a single-stranded stage of the 3′-standing segment of the second specific primer extension product that is suitable for the binding of the first primer oligonucleotide, so that a new primer extension reaction can take place. During the synthesis of the second primer extension product the activator oligonucleotide can be separated from the binding with the first primer extension product by means of strand displacement, for example by polymerase and/or by the second primer oligonucleotide.

A target sequence-specific activator oligonucleotide preferably is constructed such that it is able to contribute to the amplification of preferably one defined specific sequence variant of a pre-defined target sequence.

An allele-specific activator oligonucleotide preferably is constructed such that it is able to preferably cause the amplification of a specific sequence variant of a target sequence (e.g., allele 1), wherein with another sequence variant of the same target sequence (e.g., allele 2) amplification proceeds with a reduced efficiency or does not take place in the given time at all or results in a yield of amplification products that is not sufficient for a detection. Thus, measurable differences result during an amplification reaction that depend on whether or not the sequence of an activator oligonucleotide specific for an allele variant specifically matches with the sequence of a nucleic acid chain provided at the beginning of the reaction which is used as a start nucleic acid and comprises the target sequence with a polymorphous locus. In case of a match or perfect-match complementarity between the sequence composition of the polymorphous locus of a start nucleic acid chain and a corresponding sequence segment in the activator oligonucleotide specific characteristic amplification takes place. In case of a lacking match or mismatch complementarity between the sequence composition of the polymorphous locus of a start nucleic acid chain and a corresponding sequence segment in the activator oligonucleotide the amplification proceeds other than a specific characteristic amplification.

Thus, an allele-specific activator oligonucleotide not only is provided target sequence-specific, but also allele-specific. Thus, depending on the complexity of a polymorphous locus of a target sequence several activator oligonucleotides specific for respective allele variants of a target sequence may be constructed.

Preferably, an activator oligonucleotide comprises at least one sequence segment which has a sequence composition complementary to a specific allele variant of a target sequence. In addition, an activator oligonucleotide preferably comprises at least one sequence segment comprising a complementary sequence uniform for all sequence variants of a target sequence. In one embodiment an activator oligonucleotide comprises at least one sequence segment which can complementary bind to a variant of a polymorphous locus of a target sequence.

In one embodiment several activator oligonucleotides are provided that comprise allele-specific sequence segments.

The length of a sequence segment of an activator oligonucleotide complementary to the polymorphous locus of a start nucleic acid chain can comprise a region from one nucleotide up to 100 nucleotides, better from one nucleotide up to 50 nucleotides, preferably from one nucleotide up to 20 nucleotides. The length of at least one sequence segment of an activator oligonucleotide which is complementary to uniform sequence segments of a target sequence (first and second uniform sequence segments of a start nucleic acid) can comprise the following ranges: from 4 to 100 nucleotides, better from 6 to 100 nucleotides, preferably from 8 to 50 nucleotides.

In one embodiment a sequence segment of an activator oligonucleotide which is complementary to at least one variant of a polymorphous locus of a target sequence is located in the third region of the activator oligonucleotide.

In a further embodiment, a sequence segment of an activator oligonucleotide which is complementary to at least one variant of a polymorphous locus of a target sequence is located in the second region of the activator oligonucleotide.

In a further embodiment, a sequence segment of an activator oligonucleotide which is complementary to at least one variant of a polymorphous locus of a target sequence is located in the second and the third region of the activator oligonucleotide.

Formation of base pairs among two nucleic acid strands may be substantially complementary, that is that between 70% and 100%, better between 90% and 100%, preferably between 95% and 100% of the nucleotides of a strand can complementary bind to each other. For example, a nucleotide position in a strand of 20 nucleotides may not have a complementary bond, this corresponds to 95% complementarity.

In case of a perfect-match binding of two sequences there is preferably 100% complementary base pairing between both strands of a duplex. Thus, a perfect-match duplex comprises a mismatch between strands.

Amplification System:

An amplification system comprises at least one target sequence-specific activator oligonucleotide, at least one first primer oligonucleotide, and at least one second primer oligonucleotide as well as at least one template-dependent polymerase which is able to support a mainly specific amplification of the nucleic acid chain to be amplified using a start nucleic acid chain as a template comprising at least one characteristic target sequence or target sequence variant under suitable reaction conditions, wherein mainly target sequence-specific first primer extension products and mainly specific second primer extension products are synthesized.

An allele-specific amplification system comprises at least one allele-specific activator oligonucleotide, at least one first primer oligonucleotide, and at least one second primer oligonucleotide (depending on the design, primers may be target sequence-specific and/or allele-specific) as well as at least one template-dependent polymerase, which is able to support a mainly specific amplification of the nucleic acid chain to be amplified using a start nucleic acid chain as a template comprising at least one characteristic target sequence or target sequence variant (an allele) under suitable reaction conditions, wherein mainly specific first primer extension products and mainly specific second primer extension products are synthesized. Preferably, both primer extension products are allele-specific.

An amplification system can comprise several allele-specific amplification systems, wherein each allele-specific amplification system preferably amplifies at least one of the target sequence variants.

Strand Displacement:

This refers to a process that by action of a suitable means results in a complete or partial separation of a first double strand (for example consisting of A1 and B1 strands) and in simultaneous/parallel formation of a new second double strand, wherein at least one of the strands (A1 or B1) takes part in the formation of said new second strand. Here, distinctions can be made between two types of strand displacement.

In a first type of the strand displacement formation of a new second double strand can be by using an already existing complementary strand that at the beginning of the reaction is generally present as a single-stranded form. Here, the means of the strand displacement (for example, a pre-formed single-stranded strand C1 that has a complementary sequence to strand A1, acts on the first already formed double strand (A1 and B1) and complementary binds to strand A1, whereby strand B1 is displaced from the binding with strand A1. If the displacement of B1 proceeds completely, so the result of the C1 action is a new double strand (A1:C1) and a single-stranded strand B1. If the displacement of B1 proceeds incomplete, so the result depends on several factors. For example, a complex of partially double-stranded A1:B1 and A1:C1 can be present as an intermediate product.

In a second type of the strand displacement the formation of a new second double strand can be with a simultaneously proceeding enzymatic synthesis of the complementary strand, wherein a strand of the first pre-formed double strand is present as a template for the synthesis by the polymerase. Here, the means of the strand displacement (for example, polymerase having a strand displacement ability) acts on the already pre-formed double strand (A1 and B1) and synthesizes a new strand D1 complementary to strand A1, wherein at the same time strand B1 is displaced from the binding with strand A1.

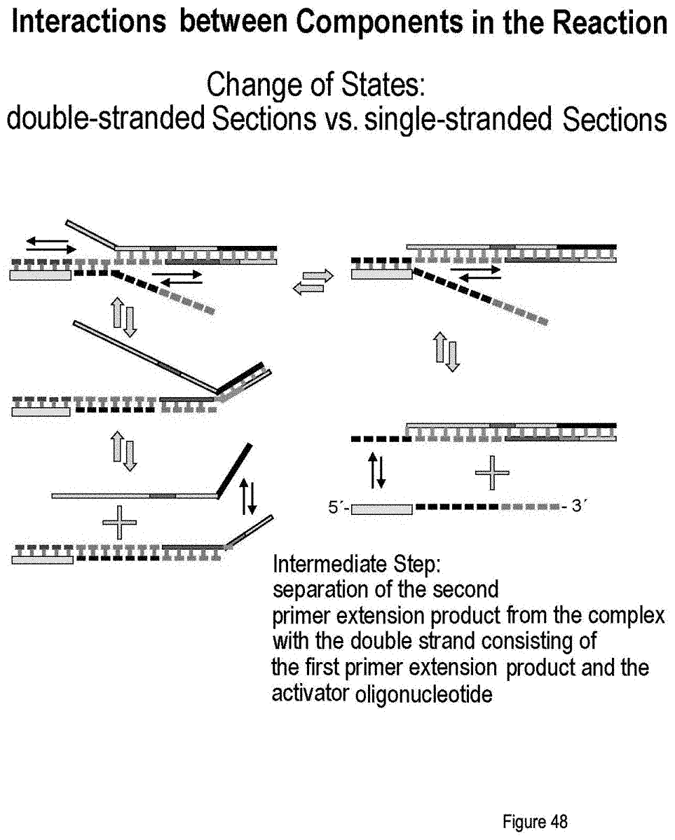

Under the term “nucleic acid-mediated strand displacement” a sum/series of intermediate steps is brought together that can be in equilibrium with each other and as a result lead to the transient or permanent opening of a first pre-formed duplex (consisting of complementary strands A1 and B1) and formation of a new second duplex (consisting of complementary strands A1 and C1), wherein A1 and C1 are complementary. Said process is illustrated in FIG. 48 (see below).

It is known that an essential structural requirement for the initiation of a strand displacement is to cause a spatial proximity between a duplex end (pre-formed first duplex of A1 and B1) and a single-stranded strand (C1) that initiates the strand displacement (wherein A1 and C1 can form a complementary strand). Such a spatial proximity can preferably be caused by means of a single-stranded overhang (in the literature examples with short overhangs are known, in English referred to as “toehold”) that complementary binds the single-stranded strand (C1) transiently or permanently, and thus brings complementary segments of strands C1 and A1 sufficiently close, so that a successful strand displacement of strand B1 can be initiated. Efficacy of the initiation of the nucleic acid-mediated strand displacement generally is the higher the closer the complementary segments of strands A1 and C1 are positioned to each other.

A further essential structural requirement of the efficient continuation of a nucleic acid-mediated strand displacement in inner segments is a high complementarity between strands (e.g., between A1 and C1) that have to form a new double strand. So, for example individual nucleotide mutations (in C1) can result in the disruption of a strand displacement (e.g., described for branch migration).

The present invention uses the ability of complementary nucleic acids for sequence-dependent nucleic acid-mediated strand displacement.

Preferred embodiments of the invention are explained in detail in the figures and examples.

FIG. 1A

schematically shows a start nucleic acid chain and components of the amplification system in the batch before the start of the amplification:

Start nucleic acid (start na) comprising a target sequence.

Primer 1 (P1.1), primer 2 (P2.1), an activator oligonucleotide (C1.1).

Components for primer extension products: polymerase (Pol) and dNTPs

FIG. 1B

schematically shows the result of the amplification:

Primer extension product P1.1-Ext starting from P1.1, primer extension product 2.1-Ext. These products (P1.1-Ext, P2.1-Ext) among themselves and with the activator oligonucleotide can form different complex forms (depending on the concentration ratio and reaction conditions). In detail, these forms can comprise complexes from P1.1-Ext/C1.1 and/or P1.1-Ext and/or P1.1-Ext/C1.1/P2.1-Ext.

P1-Ext comprises a 3′ segment that is not complementary bound by the activator oligonucleotide. Said segment acts as a binding partner for the oligonucleotide probe.

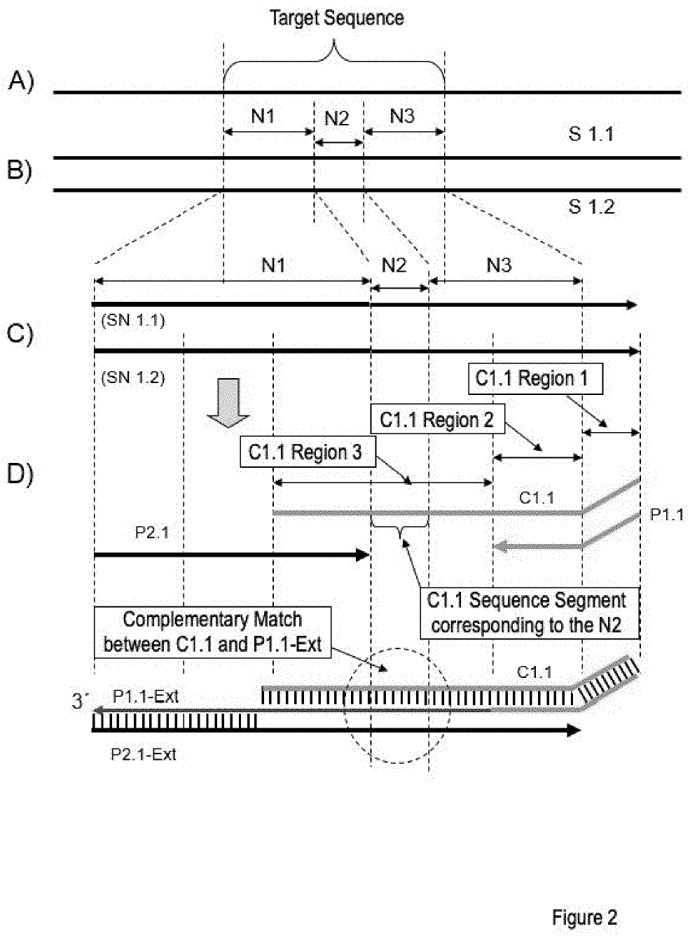

FIGS. 2A to 2C

schematically show the topography of a target nucleic acid chain with a polymorphous locus (N2) and two uniform target sequence segments (N1 and N3). The start nucleic acid chains (SN 1.1 and SN 1.2) schematically show the topography of a target nucleic acid sequence within start nucleic acids. Both start nucleic acid chains comprise target sequence segments N1 and N3 that are identical and uniform in both start nucleic acid chains. The respective sequence segment N2 is characteristic and specific with each start nucleic acid, so that both start nucleic acids can be differed this way.

In this embodiment a target sequence that can comprise several sequence variants in one polymorphous locus (N2) further comprises at least one first target sequence segment (N1) which is characteristic and uniform for all the target sequence variants of a target sequence (here, target sequence group comprising SN 1.1 and SN 1.2). Further, a target sequence comprises at least one second target sequence segment (N3) which is characteristic and uniform for all the target sequence variants of a target sequence (here, target sequence group comprising SN 1.1 and SN 1.2). Such uniform target sequence segments are preferably located on both sides of a polymorphous locus and thus, flank a polymorphous locus (with sequence variants) of a target sequence from both sides. Preferably, the first uniform target sequence segment (N1) differs from the sequence composition of the second uniform target sequence segment (N3) in its sequence composition in at least one nucleotide position.

FIG. 2D: During an amplification a nucleic acid chain to be amplified is exponentially propagated what is assisted by an activator oligonucleotide C1.1, so that a first primer (P1.1) and a second primer (P2.1) are extended and primer extension products are synthesized (P1.1-Ext and P2.1-Ext) that comprise segments N1, N2 and N3 of a target nucleic acid chain.

Here, the activator oligonucleotide comprises a sequence segment corresponding to the polymorphous locus of a target sequence. The sequence composition of said sequence segment of the activator oligonucleotide is characteristic and specific for one of the sequence variants of the target sequence within said locus, so that said segment can complementary bind to the corresponding segment of the first primer extension product which results for example from the copy process of a specific start nucleic acid chain. In this way, the activator oligonucleotide can contribute to a strand separation specific for said sequence variant which results in an amplification.

In this embodiment the activator oligonucleotide is arranged such that a sequence segment of the activator oligonucleotide corresponding to the N2 of the target sequence is in its third region and does not overlap with any of the primer sequences.

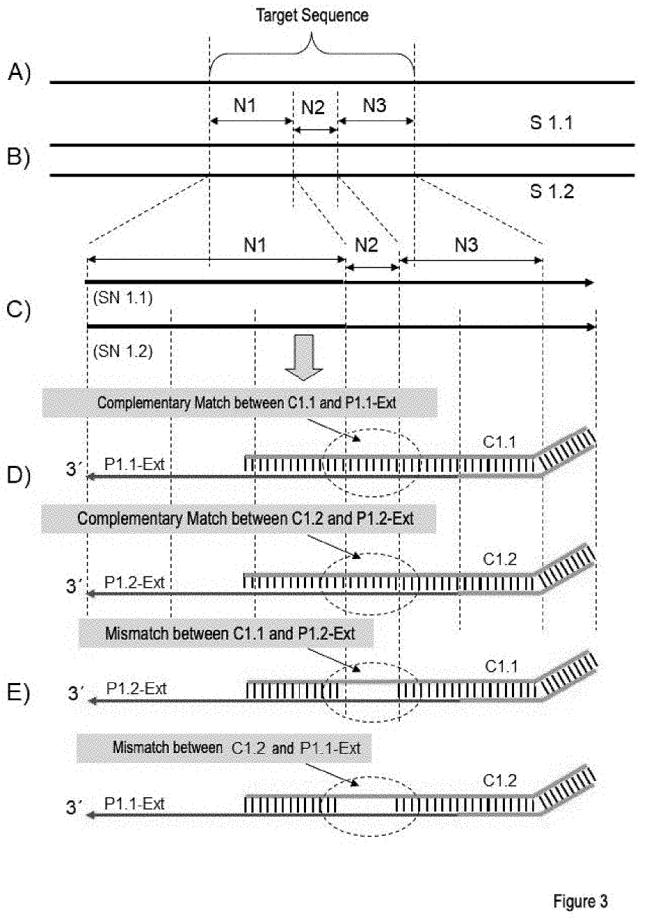

FIG. 3 (A-C)

schematically shows the topography of a target nucleic acid chain with a polymorphous locus (N2) and two uniform target sequence segments (N1 and N3) as illustrated in FIG. 2. FIG. 3D shows the result of the use of two activator oligonucleotides that are characteristic and specific for one sequence variant each (C1.1 and C1.2), wherein each of the activator oligonucleotides comprises a sequence segment corresponding to the polymorphous locus of the target sequence (N2), wherein C1.1 and C1.2 in this sequence segment are different. This results in a specific and characteristic pairing of a specific and characteristic sequence variant (an allele variant) of a specific target sequence and an activator oligonucleotide characteristic and specific for said sequence variant.

The specific activator oligonucleotides are able to form a complementary strand only with one strand of a nucleic acid chain to be amplified (with the respective first primer extension product) comprising only one specific sequence variant each in which the segment of the activator oligonucleotide corresponding to the N2 can form a perfect-match double strand with the first primer extension product (C1.1 with P1.1-Ext and C1.2 with P1.2-Ext) and thus, contribute to the strand separation.

FIG. 3E schematically shows a construct comprising C1.1 and P1.2-Ext or C1.2 and P1.1-Ext. In such duplexes a mismatch between an activator oligonucleotide and a first primer extension product would be formed, namely in the sequence segment corresponding to the locus N2 of the target sequence. Such mismatch including complexes primarily are formed in the absence of a competitor strand (in this case the complementary P1.1-Ext or P1.2-Ext, respectively is lacking). For this reason, in the absence of a second complementary primer extension product a duplex is formed between the 5′ end of an activator oligonucleotide and corresponding portions of the first primer extension product.

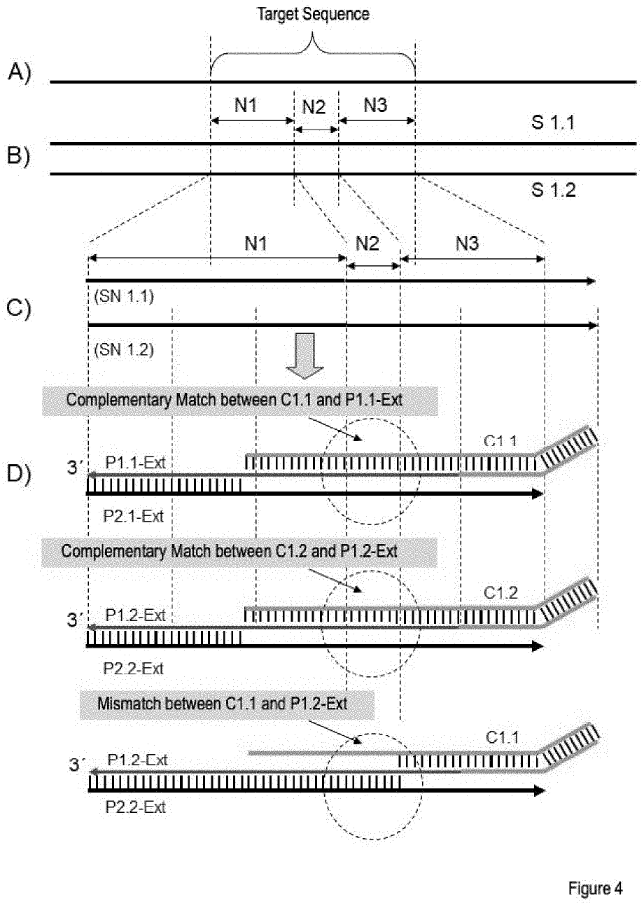

FIG. 4D schematically groups which effect the presence of the fully complementary strand has on the possible mismatch duplex of FIG. 3E: Due to the fully complementary nature of the primer extension product synthesized by a polymerase both primer extension products formed during the synthesis each comprise fully complementary sequences. Binding of such fully complementary sequences to each other is more effective than with sequences which comprise a mismatch. For that reason, in the absence of a second complementary primer extension product no duplex is formed between the 5′ end of an activator oligonucleotide and corresponding portions of the first primer extension product. Thus, strand separation is only over a shorter distance and less effective than with fully complementary strands. This results in a deceleration or even interruption of an amplification.

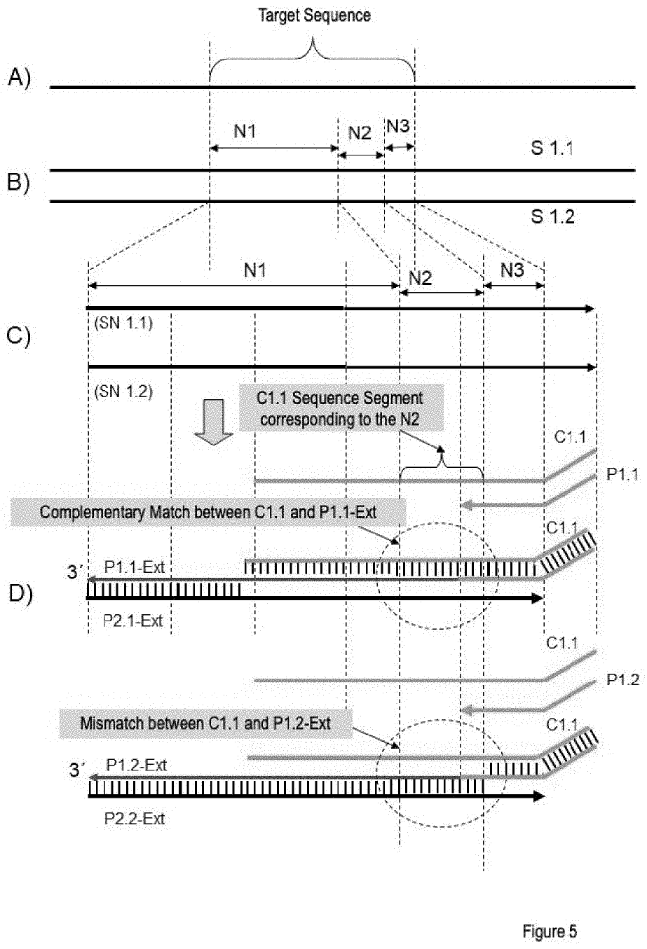

FIG. 5 schematically shows another topography of an N2 of a target nucleic acid chain with respect to an activator oligonucleotide and the first primer.

In this example the sequence segment of the activator oligonucleotide corresponding to the N2 both partially comprises the third region and partially the second region. Also, the 3′ segment of the first primer is designed sequence-specific and characteristic for one specific and characteristic sequence variant of the target nucleic acid. That is, both activator oligonucleotide and first primer oligonucleotide take part in the allele discrimination. Thus, for each specific sequence variant of the polymorphous locus both a specific activator oligonucleotide and a specific first primer oligonucleotide can be constructed.

By using specific and characteristic primers as well as suitable reaction conditions primer extension products are synthesized in a specific manner, so that P1.1 is specifically extended and this results in a P1.1-Ext, and P1.2 is specifically extended and this results in P1.2-Ext. Because the second regions of P1.1 and P1.2 are designed the same, C1.1 can bind to P1.2-Ext and certainly, strand displacement can be initiated, but a complete strand separation cannot take place, since P1.2-Ext contains no fully complementary sequence segment in the sequence segment corresponding to N2.

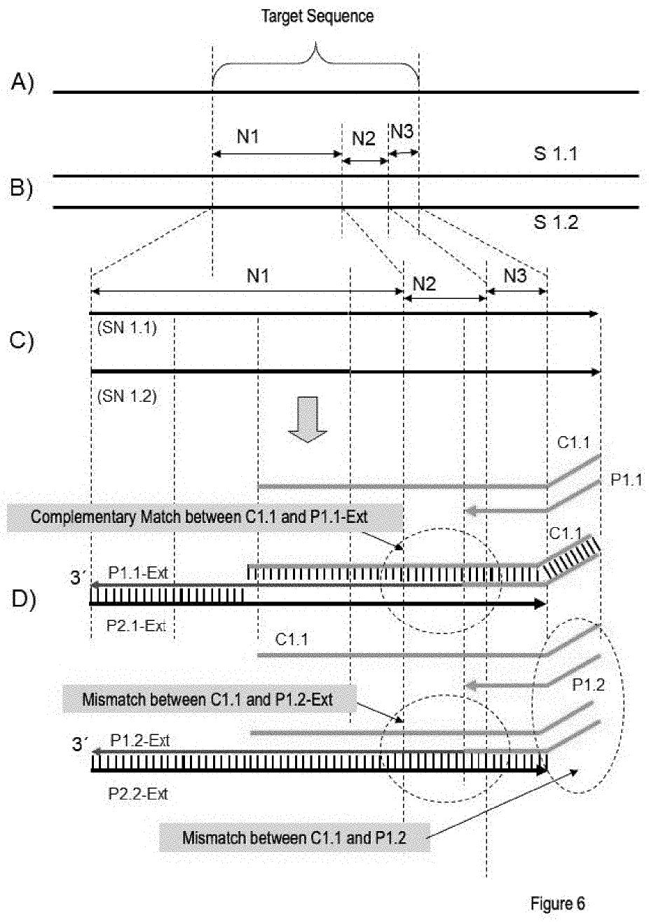

FIG. 6 schematically shows another topography of an N2 of a target nucleic acid chain with respect to an activator oligonucleotide and the first primer, wherein interaction between a specific P1.2-Ext and a specific C1.1 can be reduced by the sequence-specific coding of the complementary portions of the second primer region to the corresponding complementary portions of the first region of the activator oligonucleotide. This results in the possibility for multiple multiplexing, wherein each pair comprising at least one first primer oligonucleotide and the activator oligonucleotide comprises a sequence composition of a duplex that is characteristic for them and comprises the first region of the activator oligonucleotide and the second region of the first primer.

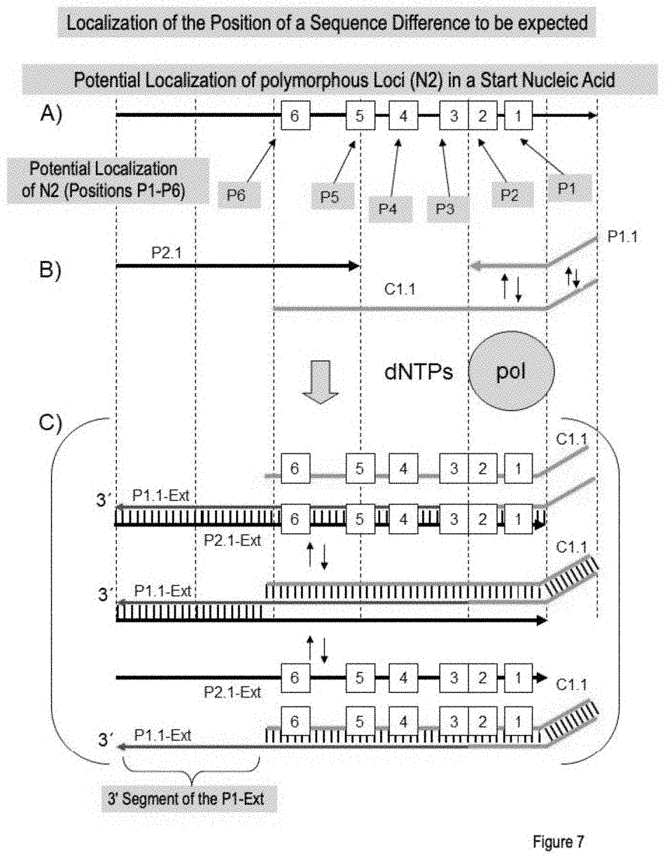

FIG. 7 schematically shows potential positions of a polymorphous locus within a target sequence (positions 1 to 6) and their corresponding sequence segment positions in individual components of an amplification system. The polymorphous loci of a target sequence schematically illustrated here comprise 2 to 50 nucleotides, better 4 to 30 nucleotides, that are characteristic and specific for an allele variant of a target sequence.

Altogether, arrangement of individual amplification components may be designed such that several variants/combinations are possible:

Arrangement 1 (P1): polymorphous locus (N2) of the target sequence overlaps the first primer (first region) and activator oligonucleotide (second region). Thus, these components of the amplification system may be constructed specific and characteristic for respective sequence variants of the target sequence. In this arrangement a target sequence-specific second primer is used which however is not sequence variant-specific.

Arrangement 2 (P2): polymorphous locus (N2) of the target sequence overlaps the first primer (first region) and activator oligonucleotide (second region). Thus, these components of the amplification system may be constructed specific and characteristic for respective sequence variants of the target sequence. In this arrangement a target sequence-specific second primer is used which however is not sequence variant-specific. Arrangement P2 mainly differs from P1 in that N2 mainly is in the 3′-terminal sequence segment of the first primer. In this way, optionally better specificity of the amplification may be achieved.

Arrangement 3 (P3): polymorphous locus (N2) of the target sequence only overlaps the activator oligonucleotide (third region), wherein the corresponding sequence segment of the activator oligonucleotide is in the 5′ segment of the synthesized portion of the first primer extension product. Thus, the activator oligonucleotide of the amplification system may be constructed specific and characteristic for respective sequence variants of the target sequence. In this arrangement target sequence-specific first and second primers are used that however are not sequence variant-specific. Due to the possible proximity of the second blocking unit binding of the activator oligonucleotide to the first primer extension product may be affected by nucleotide modifications of the second blocking unit.

Arrangement 4 (P4): polymorphous locus (N2) of the target sequence only overlaps the activator oligonucleotide (third region). Thus, the activator oligonucleotide of the amplification system may be constructed specific and characteristic for respective sequence variants of the target sequence. In this arrangement target sequence-specific first and second primers are used that however are not sequence variant-specific. This sequence segment of the activator oligonucleotide is in 5′ direction from the second blocking unit and can comprise several DNA nucleotide monomers, e.g., from 5 to 30.

Arrangement 5 (P5): polymorphous locus (N2) of the target sequence overlaps the second primer and activator oligonucleotide (third region). Thus, these components of the amplification system may be constructed specific and characteristic for respective sequence variants of the target sequence. In this arrangement a target sequence-specific first primer is used which however is not sequence variant-specific.

Arrangement 6 (P6): polymorphous locus (N2) of the target sequence overlaps the second primer and activator oligonucleotide (third region, close to the 5′ end). Thus, these components of the amplification system may be constructed specific and characteristic for respective sequence variants of the target sequence. In this arrangement a target sequence-specific first primer is used which however is not sequence variant-specific.

The different designs of individual elements also result in different primer extension products synthesized during an amplification (FIG. 7C).

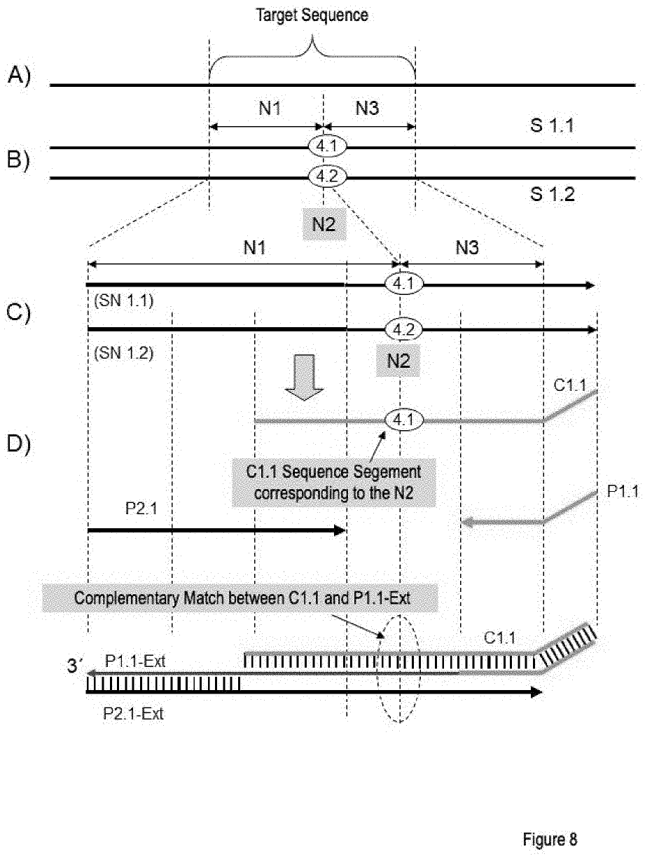

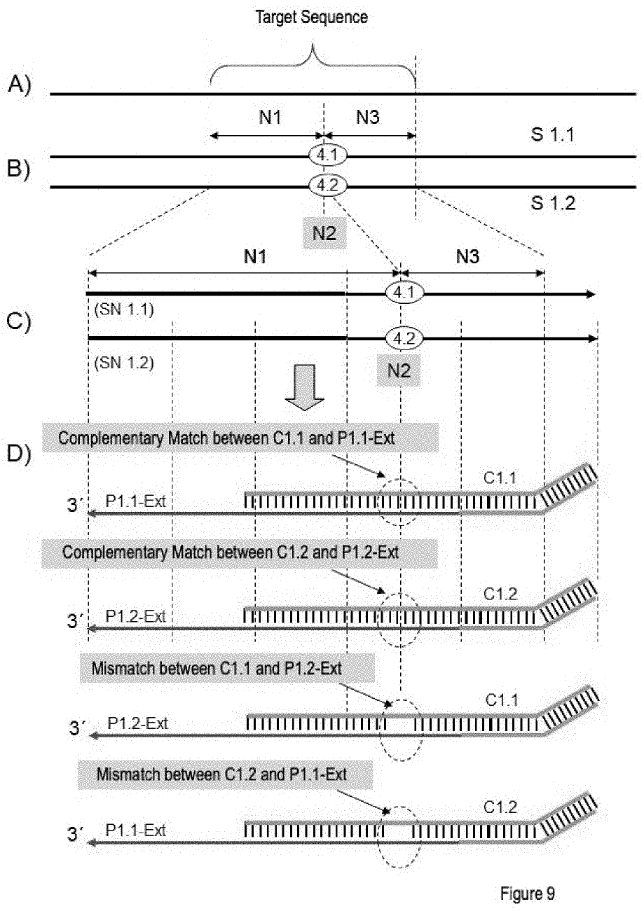

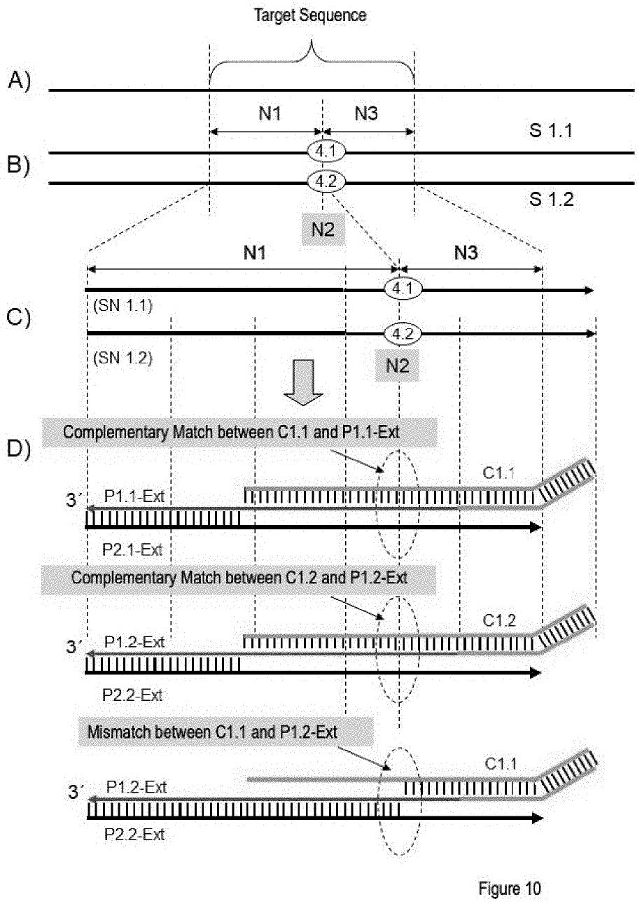

FIGS. 8-10 schematically show the topography of a target nucleic acid chain with a polymorphous locus (N2) and two uniform target sequence segments (N1 and N3), as illustrated in FIG. 2, wherein the polymorphous locus only comprises one nucleotide position (e.g., an SNV or an SNP or a point mutation, etc.). Thus, the differences between individual sequence variants are only in one nucleotide (here designated with 4.1 and 4.2). Such a locus (N2) can result in similar arrangements of individual components as a locus having at least 2 nucleotides. Thus, when the activator oligonucleotide binds to a complementary first primer extension product (perfect-match) an amplification can take place. When a mismatch is formed, amplification is inhibited or decelerated or even interrupted. Like with longer N2 in case of single nucleotide mismatches between the activator oligonucleotide and a first primer extension product strand separation may be interrupted/decelerated.

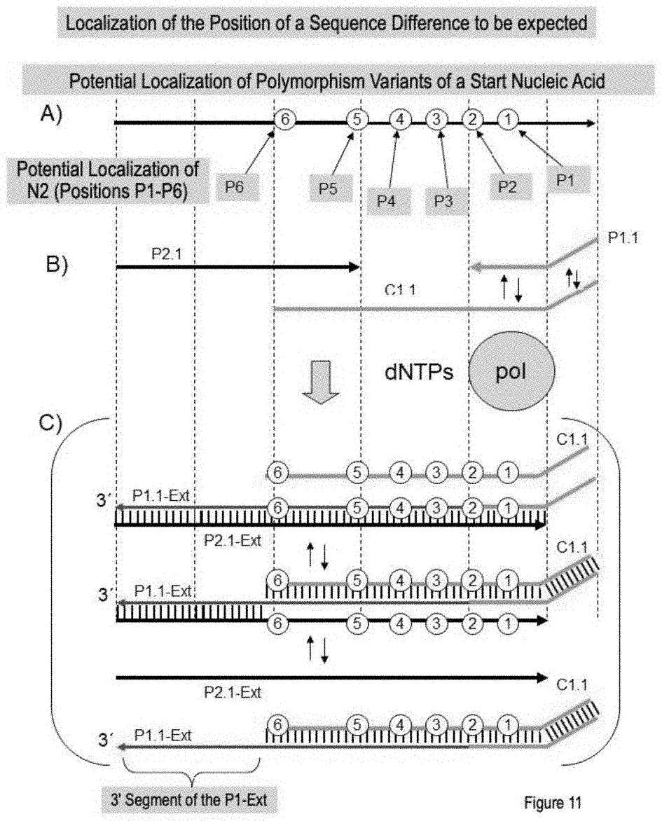

FIG. 11 schematically shows potential positions of a polymorphous locus having sequence variance of only one nucleotide within a target sequence (position 1 to 6) and its corresponding sequence segment-positions in individual components of an amplification system.

Altogether, arrangement of individual amplification components may be designed such that several variants/combinations are possible:

Arrangement 1 (P1): polymorphous locus (N2) of the target sequence overlaps the first primer (first region) and activator oligonucleotide (second region). Thus, these components of the amplification system may be constructed specific and characteristic for respective sequence variants of the target sequence. In this arrangement a target sequence-specific second primer is used which however is not sequence variant-specific.

Arrangement 2 (P2): polymorphous locus (N2) of the target sequence overlaps the first primer (first region) and activator oligonucleotide (second region). Thus, these components of the amplification system may be constructed specific and characteristic for respective sequence variants of the target sequence. In this arrangement a target sequence-specific second primer is used which however is not sequence variant-specific. Arrangement P2 mainly differs from P1 in that N2 mainly can be in the 3′-terminal sequence segment of the first primer or even comprises the 3′-terminal nucleotide. In this way, specificity of the amplification may optionally be further increased.

Arrangement 3 (P3): polymorphous locus (N2) of the target sequence only overlaps the activator oligonucleotide (third region), wherein the corresponding sequence segment of the activator oligonucleotide is in the 5′ segment of the synthesized portion of the first primer extension product. Thus, the activator oligonucleotide of the amplification system may be constructed specific and characteristic for respective sequence variants of the target sequence. In this arrangement target sequence-specific first and second primers are used that however are not sequence variant-specific. Due to the possible proximity of the second blocking unit binding of the activator oligonucleotide to the first primer extension product may be affected by nucleotide modifications of the second blocking unit.

Arrangement 4 (P4): polymorphous locus (N2) of the target sequence only overlaps the activator oligonucleotide (third region). Thus, the activator oligonucleotide of the amplification system may be constructed specific and characteristic for respective sequence variants of the target sequence. In this arrangement target sequence-specific first and second primers are used that however are not sequence variant-specific. This sequence segment of the activator oligonucleotide is in 5′ direction from the second blocking unit. This segment of the activator oligonucleotide can comprise several DNA nucleotide monomers, e.g., from 5 to 30, wherein the N2-corresponding sequence segment may be flanked by at least 3 to 15 DNA nucleotide building blocks on both sides.

Arrangement 5 (P5): polymorphous locus (N2) of the target sequence overlaps the second primer and activator oligonucleotide (third region). Thus, these components of the amplification system may be constructed specific and characteristic for respective sequence variants of the target sequence. The arrangement may be designed such that the 3′-terminal nucleotide of the second primer corresponds to the N2 locus. In this arrangement a target sequence-specific first primer is used that however is not sequence variant-specific.

Arrangement 6 (P6): polymorphous locus (N2) of the target sequence overlaps the second primer and activator oligonucleotide (third region, close to the 5′ end). Thus, these components of the amplification system may be constructed specific and characteristic for respective sequence variants of the target sequence. In this arrangement a target sequence-specific first primer is used which however is not sequence variant-specific.

The different designs of individual elements also result in different primer extension products synthesized during an amplification (FIG. 11C).

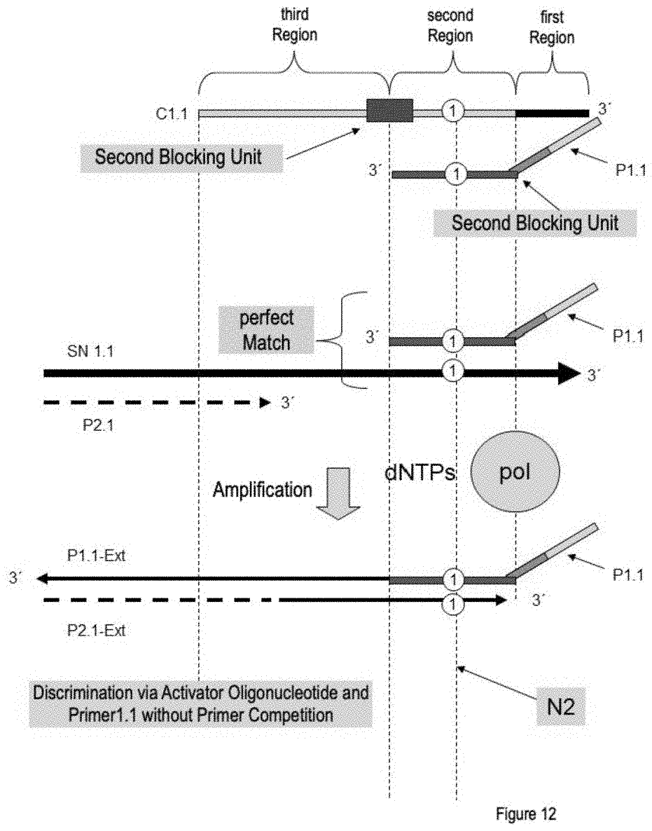

FIG. 12 schematically shows an embodiment with the topography of a specific amplification system and a start nucleic acid chain having a polymorphous locus (N2) in an embodiment in which the segment of the activator oligonucleotide corresponding to N2 is in the second region. A specific P1.1 under reaction conditions preferably may specifically bind to the complementary position of a specific sequence variant of the start nucleic acid (SN 1.1) and initiate the synthesis of the P1.1-Ext. Thereby, P1.1-Ext and P2.1-Ext are formed during the amplification. No competitor primer is used.

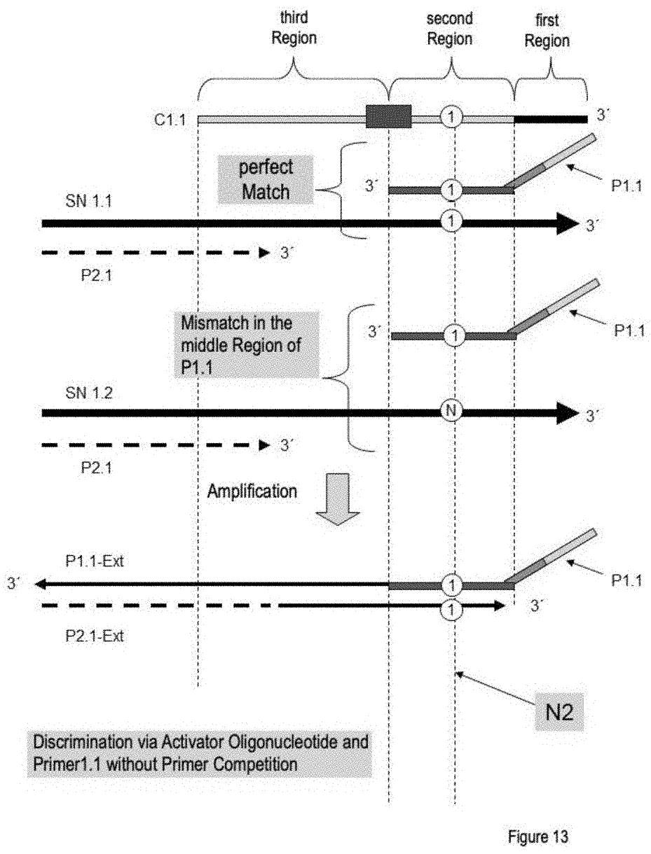

FIG. 13 schematically shows an embodiment with the topography of a specific amplification system and a start nucleic acid chain having a polymorphous locus (N2) in an embodiment in which the segment of the activator oligonucleotide corresponding to N2 is in the second region. A specific P1.1 under reaction conditions preferably may specifically bind to the complementary position of a specific sequence variant of the start nucleic acid (SN 1.1) and initiate the synthesis of the P1.1-Ext. Thereby, P1.1-Ext and P2.1-Ext are formed during the amplification. No competitor primer is used. Binding to another sequence variant (SN 1.2) is less efficient due to the mismatch with position (N) within the primer binding site of SN 1.2. Thus, amplification is started less efficient.

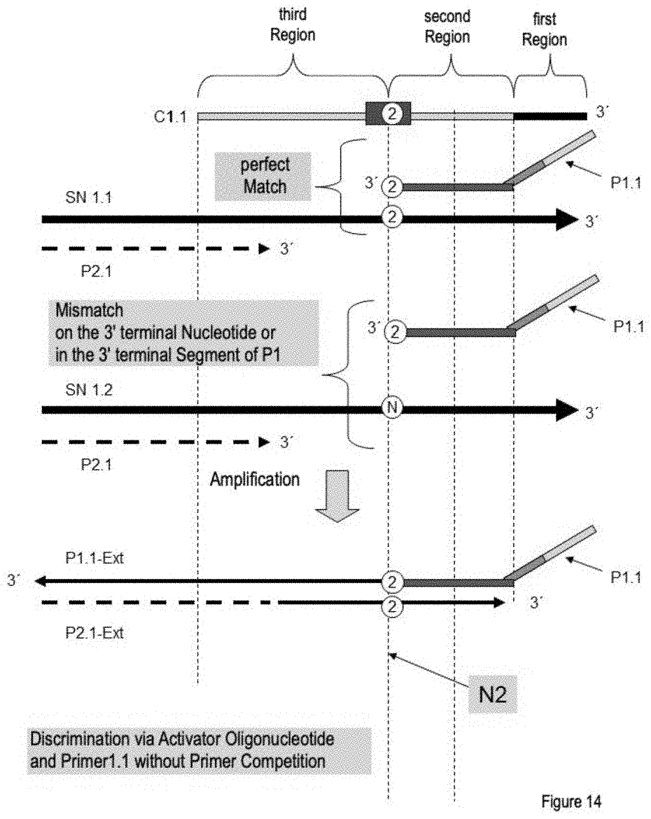

FIG. 14 schematically shows an embodiment with the topography of a specific amplification system and a start nucleic acid chain having a polymorphous locus (N2) in an embodiment in which the segment of the activator oligonucleotide corresponding to N2 is in the second region (2). N2 locus also comprises the 3′-terminal nucleotide and/or the 3′-terminal segment of the first primer. A specific P1.1 under reaction conditions preferably may specifically bind to the complementary position of a specific sequence variant of the start nucleic acid (SN 1.1) and initiate the synthesis of the P1.1-Ext. Thereby, P1.1-Ext and P2.1-Ext are formed during the amplification. No competitor primer is used. Binding and initiation of the primer extension to another sequence variant (SN 1.2) is less efficient due to the mismatch with position (N) within the primer binding site of SN 1.2. Thus, amplification is started less efficient.

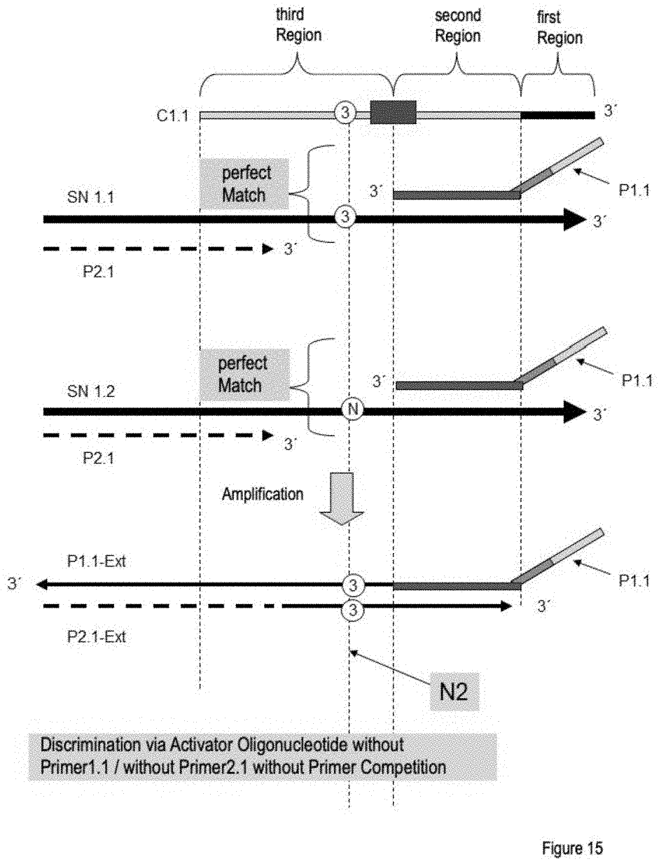

FIG. 15 schematically shows an embodiment with the topography of specific amplification system and a start nucleic acid chain having a polymorphous locus (N2) in an embodiment in which the segment of the activator oligonucleotide corresponding to N2 is in the third region (3). Target sequence-specific but not sequence variant-specific first and second primers are used. A P1.1 and P2.1 uniform for all the sequence variants of a target nucleic acid can bind to the start nucleic acid chain and initiate the synthesis of the P1.1-Ext or P2.1-Ext, respectively. Thereby, P1.1-Ext and P2.1-Ext are formed during the amplification. No competitor primer is used. Separation of both synthesized complementary primer extension products P1.1-Ext and P2.1-Ext preferably takes place specifically by complementary binding of the P1.1-Ext to the activator oligonucleotide. Separation of strands of another sequence variant that was synthesized using (SN 1.2) is less efficient due to the mismatch with position (N) to the corresponding sequence segment of the activator oligonucleotide. Thus, amplification of SN 1.2 sequence variant is less efficient or only marginal or not at all. The position of the sequence segment (3) corresponding to N2 of the target sequence is close to the second blocking unit, so that interaction between the activator oligonucleotide and the respective first primer extension product can be affected by modifications in the second blocking unit that are used.

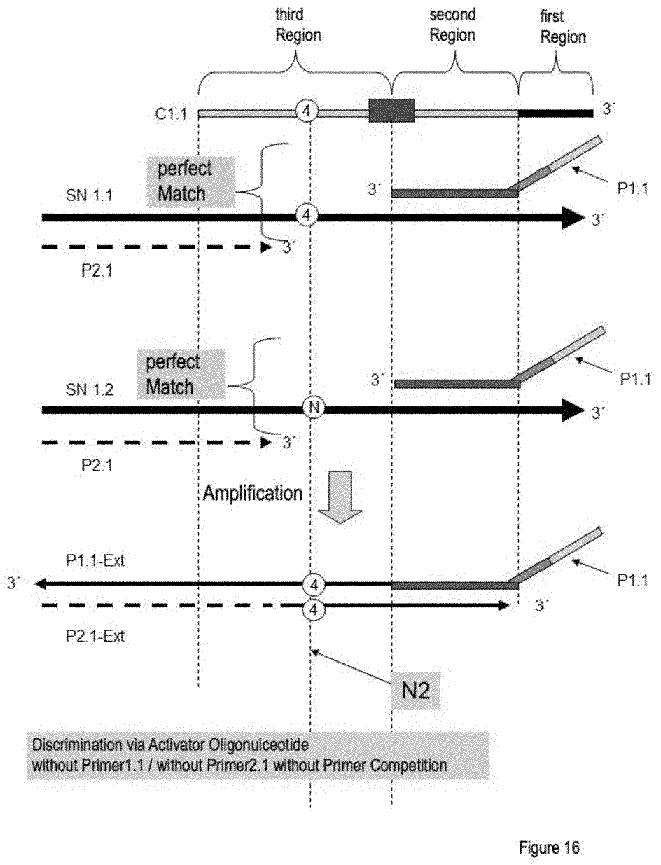

FIG. 16 schematically shows an embodiment with the topography of specific amplification system and a start nucleic acid chain having a polymorphous locus (N2) in an embodiment in which the segment of the activator oligonucleotide corresponding to N2 is in the third region (4). Target sequence-specific, but not sequence variant-specific first and second primers are used. A P1.1 and P2.1 uniform for all the sequence variants of a target nucleic acid can bind to the start nucleic acid chain and initiate the synthesis of the P1.1-Ext or P2.1-Ext, respectively. Thereby, P1.1-Ext and P2.1-Ext are formed during the amplification. No competitor primer is used. Separation of both synthesized complementary primer extension products P1.1-Ext and P2.1-Ext preferably takes place specifically by complementary binding of the P1.1-Ext to the activator oligonucleotide. Separation of strands of another sequence variant that was synthesized using (SN 1.2) is less efficient due to the mismatch with position (N) to the corresponding sequence segment of the activator oligonucleotide. Thus, amplification of SN 1.2 sequence variant is less efficient or only marginal or not at all. The position of the sequence segment (4) corresponding to N2 of the target sequence is located at a distance from the second blocking unit, so that interaction between the activator oligonucleotide and the respective first primer extension product is substantially unaffected by the used modifications in the second blocking unit. The segment of the activator oligonucleotide that corresponds to N2 preferably consists of DNA monomers, wherein between 4 and 20 DNA monomers are used and at least 4 to 10 DNA monomers are located around position 4 on both sides.

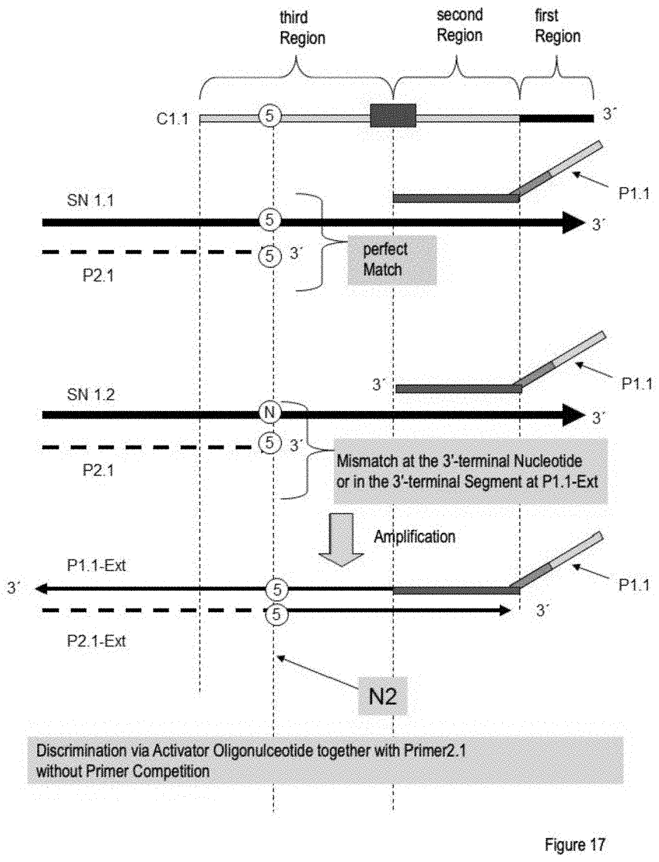

FIG. 17 schematically shows an embodiment with the topography of a specific amplification system and a start nucleic acid chain having a polymorphous locus (N2) in an embodiment in which the segment of the activator oligonucleotide corresponding to N2 is in the third region (5). The N2 locus also comprises the 3′-terminal nucleotide of the second primer. A specific P2.1 having a complementary 3′-terminal nucleotide under reaction conditions can preferably specifically bind to the complementary position of a specific sequence variant starting from strands complementary to start nucleic acid (SN 1.1) and preferably specifically initiate the synthesis of the P2.1-Ext. Thereby, P1.1-Ext and P2.1-Ext are formed during the amplification. No competitor primer is used. Binding to another sequence variant (SN 1.2) is less efficient due to the mismatch with position (N) within the primer binding site of the SN 1.2. Thus, amplification is started less efficient.

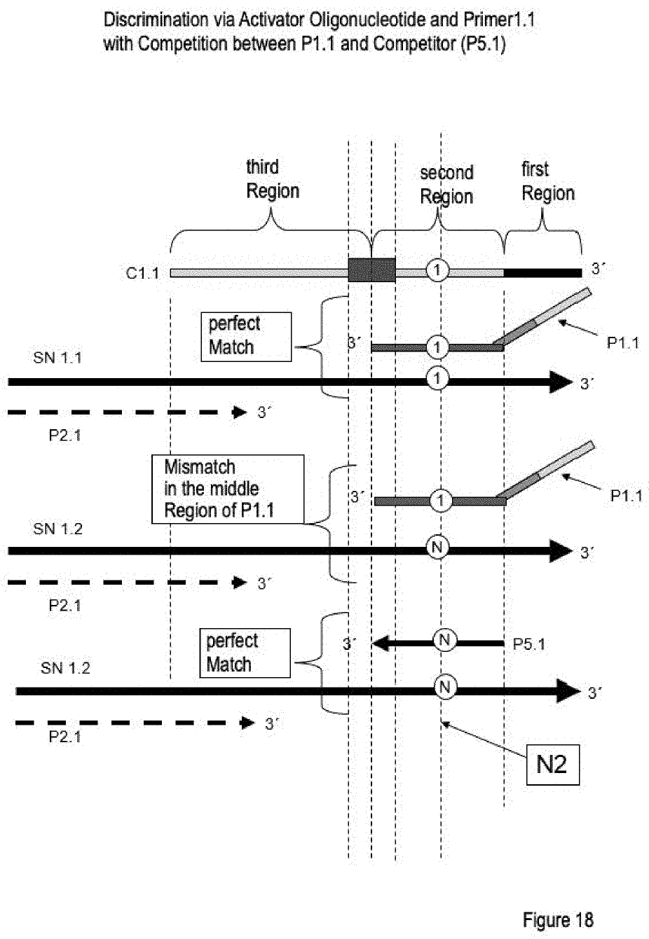

FIG. 18 schematically shows an embodiment with the topography of a specific amplification system and a start nucleic acid chain having a polymorphous locus (N2) in an embodiment, in which the segment of the activator oligonucleotide corresponding to N2 is in the second region. A specific P1.1 under reaction conditions preferably may specifically bind to the complementary position of a specific sequence variant of the start nucleic acid (SN 1.1) and initiate the synthesis of the P1.1-Ext. Thereby, P1.1-Ext and P2.1-Ext are formed during the amplification. Binding to another sequence variant (SN 1.2) is less efficient due to the mismatch with position (N) within the primer binding site of SN 1.2. Thus, amplification is started less efficient.

To increase the specificity a competitor primer (P 5.1) is added to the reaction that is able to mainly complementary bind to the sequence variant of the target sequence the amplification of which has to be suppressed. Due to a complementary binding with such primer binding sites a competitor primer may preferably bind and be extended by polymerase. The thus resulting product blocks the single-stranded primer binding sites for an interaction of the first amplification primer. In one embodiment, the 3′ end of a competitor primer binds within the second blocking unit of the activator oligonucleotide, so that no extension of said primer on the activator oligonucleotide may take place. The competitor primer comprises no second region and thus, cannot interact with the first region of the activator oligonucleotide. In this way it is prevented that the extension product of the competitor oligonucleotide is detached by a template with the help of an activator oligonucleotide.

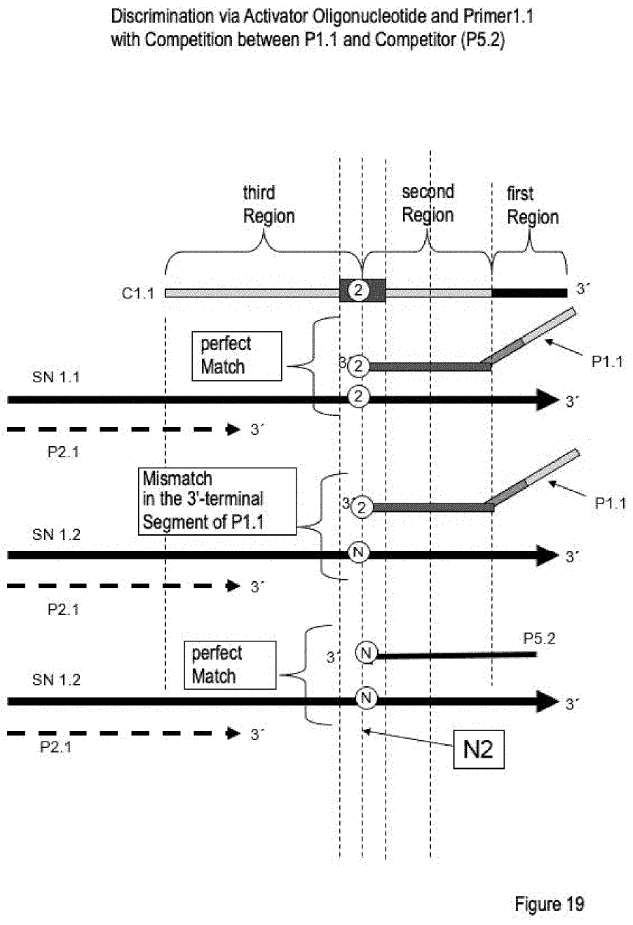

FIG. 19 schematically shows an embodiment with the topography of a specific amplification system and a start nucleic acid chain having a polymorphous locus (N2) in an embodiment in which the segment of the activator oligonucleotide corresponding to N2 is in the second region (2). The N2 locus also comprises the 3′-terminal nucleotide of the first primer. A specific P1.1 having a complementary 3′-terminal nucleotide under reaction conditions can preferably specifically bind to the complementary position of a specific sequence variant of the start nucleic acid (SN 1.1) and initiate the synthesis of the P1.1-Ext. Thereby, P1.1-Ext and P2.1-Ext are formed during the amplification. Binding to another sequence variant (SN 1.2) and extension by a polymerase is less efficient due to the mismatch with position (N) within the primer binding site of the SN 1.2. Thus, amplification is started less efficient.

To increase the specificity a competitor primer (P 5.2) is added to the reaction that is able to mainly complementary bind to the sequence variant of the target sequence the amplification of which has to be suppressed. Due to a complementary binding with such a primer binding site a competitor primer may preferably bind to certain sequence variants (here, designated with N) and extended by polymerase. The thus resulting product blocks the single-stranded primer binding site for an interaction of the first amplification primer.

In one embodiment the 3′ end of a competitor primer binds within the second blocking unit of the activator oligonucleotide, so that no extension of said primer on the activator oligonucleotide may take place. The competitor primer comprises no second region and thus, cannot interact with the first region of the activator oligonucleotide. In this way it is prevented that the extension product of the competitor oligonucleotide is detached by a template.

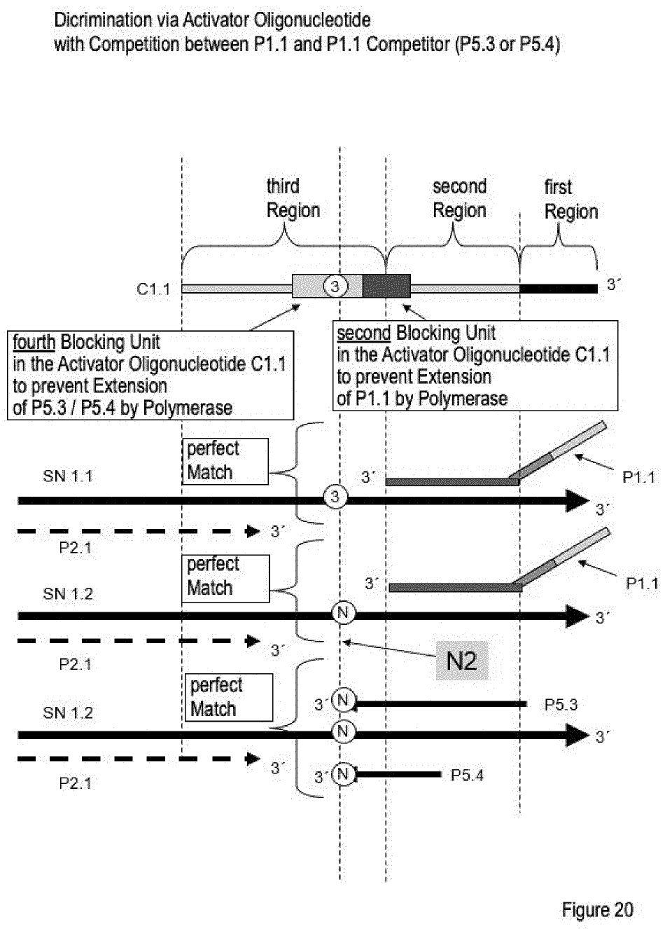

FIG. 20 schematically shows an embodiment with the topography of a specific amplification system and a start nucleic acid chain having a polymorphous locus (N2) in an embodiment in which the segment of the activator oligonucleotide corresponding to N2 is in the third region (3). Target sequence-specific, but not sequence variant-specific first and second primers are used. A P1.1 and P2.1 uniform for all the sequence variants of a target nucleic acid can bind to the start nucleic acid chain and initiate the synthesis of the P1.1-Ext or P2.1-Ext, respectively. Thereby, P1.1-Ext and P2.1-Ext are formed during the amplification. Separation of both synthesized complementary primer extension products P1.1-Ext and P2.1-Ext preferably takes place specifically by complementary binding of the P1.1-Ext to the activator oligonucleotide. Separation of strands of another sequence variant that was synthesized using (SN 1.2) is less efficient due to the mismatch with position (N) to the corresponding sequence segment of the activator oligonucleotide. Thus, amplification of SN 1.2 sequence variant is less efficient or only marginal or not at all. The position of the sequence segment (3) corresponding to N2 of the target sequence is close to the second blocking unit, so that interaction between the activator oligonucleotide and the respective first primer extension product can be affected by modifications in the second blocking unit that are used.

To increase the specificity a competitor primer (P 5.3 or P 5.4) is added to the reaction that is able to mainly complementary bind to the sequence variant of the target sequence (N) the amplification of which has to be suppressed. Due to a complementary binding with such primer binding sites a competitor primer may preferably bind to certain sequence variants (here, designated with N) and extended by polymerase. The thus resulting product blocks the single-stranded primer binding site for an interaction of the first amplification primer.

In one embodiment the competitor primer oligonucleotide (P5.3) is longer than the first region of the first primer oligonucleotide, so that its 3′ end binds within the fourth blocking unit of the activator oligonucleotide (the fourth blocking unit is made up in analogy to the second blocking unit and blocks a primer extension at the activator oligonucleotide), so that no extension of said primer on the activator oligonucleotide may take place. The competitor primer can completely (P5.3) or partially (P5.4) cover the segment of the template that can mainly complementary bind P1.1. The competitor primer comprises no second region and thus, cannot interact with the first region of the activator oligonucleotide. In this way it is prevented that the extension product of the competitor oligonucleotide is detached by a template with the help of an activator oligonucleotide.

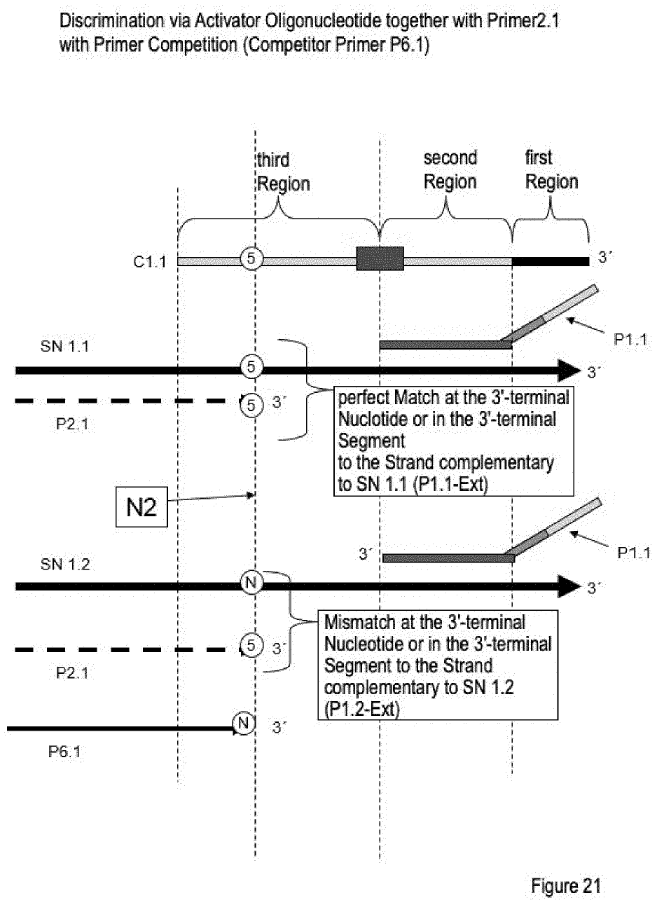

FIG. 21 schematically shows an embodiment with the topography of a specific amplification system and a start nucleic acid chain having a polymorphous locus (N2) in an embodiment in which the segment of the activator oligonucleotide corresponding to N2 is in the third region (5). The N2 locus also comprises the 3′-terminal nucleotide of the second primer. A specific P2.1 having a complementary 3′-terminal nucleotide under reaction conditions can preferably specifically bind to the complementary position of a specific sequence variant starting from strands complementary to start nucleic acid (SN 1.1) and preferably specifically initiate the synthesis of the P2.1-Ext. Thereby, P1.1-Ext and P2.1-Ext are formed during the amplification. Binding to another sequence variant (SN 1.2) is less efficient due to the mismatch with position (N) within the primer binding site of the SN 1.2. Thus, amplification is started less efficient.

To increase the specificity a competitor primer (P 6.1) is added to the reaction that is able to mainly complementary bind to the sequence variant of the target sequence the amplification of which has to be suppressed. Due to a complementary binding with such primer binding sites a competitor primer may preferably bind to certain sequence variants (here, designated with N) and extended by polymerase. The thus resulting product blocks the single-stranded primer binding sites for an interaction of the first amplification primer.

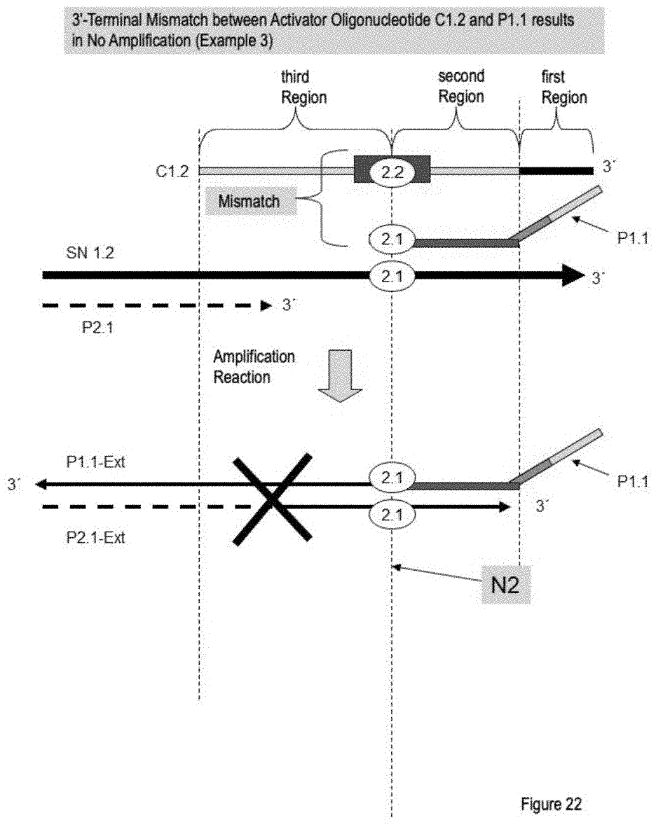

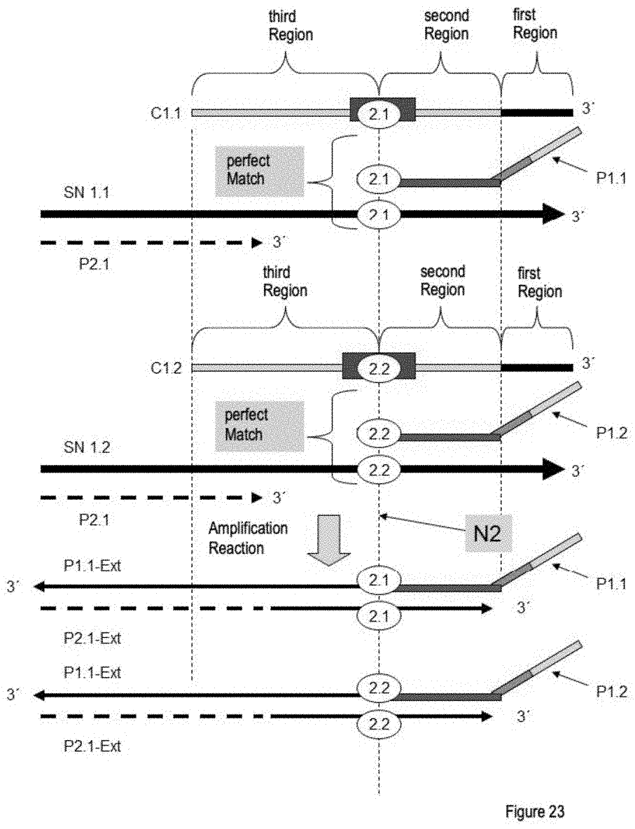

FIGS. 22-23 schematically show an embodiment and the requirement of a match in the topography of a specific amplification system:

In case of a mismatch between a first primer (here, P1.1 with perfect-match position to the template) and an activator oligonucleotide differing in the sequence (here, C1.2 with a mismatch to the 3′ end of the first primer) there is no exponential amplification. Strand displacement with each interaction between the primer and the activator oligonucleotide is hindered by such a mismatch, even though the first primer extension is successful.

This results in specific combinations (FIG. 23) between a specific and characteristic primer and a specific and characteristic activator oligonucleotide.

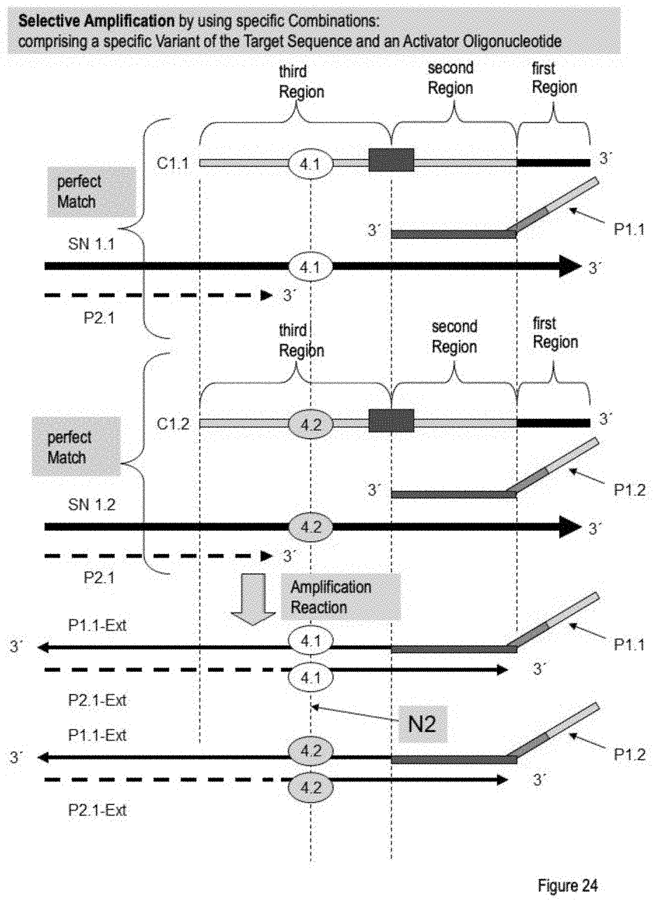

FIG. 24 schematically shows a selective amplification of several sequence variants using several selective activator oligonucleotides.

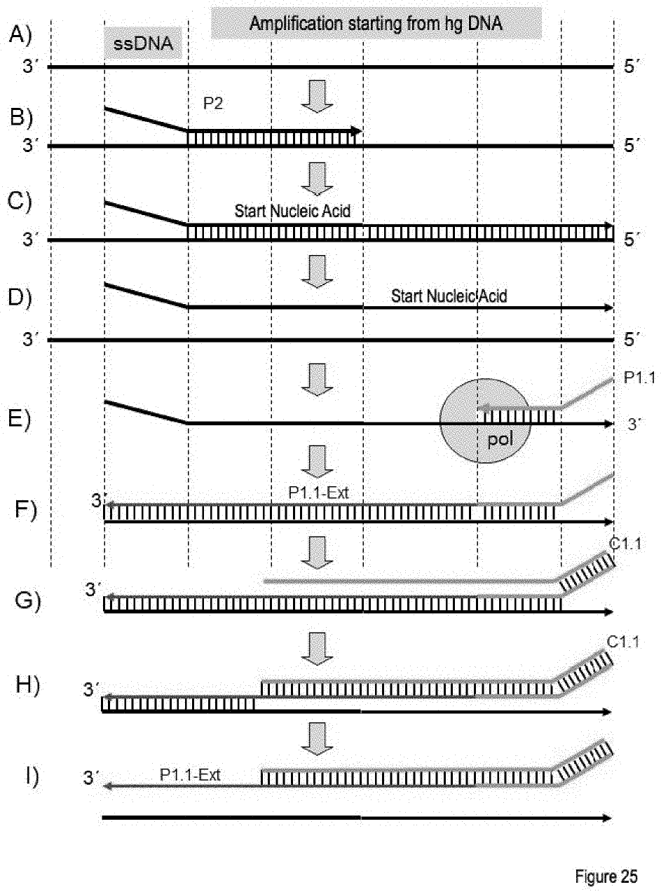

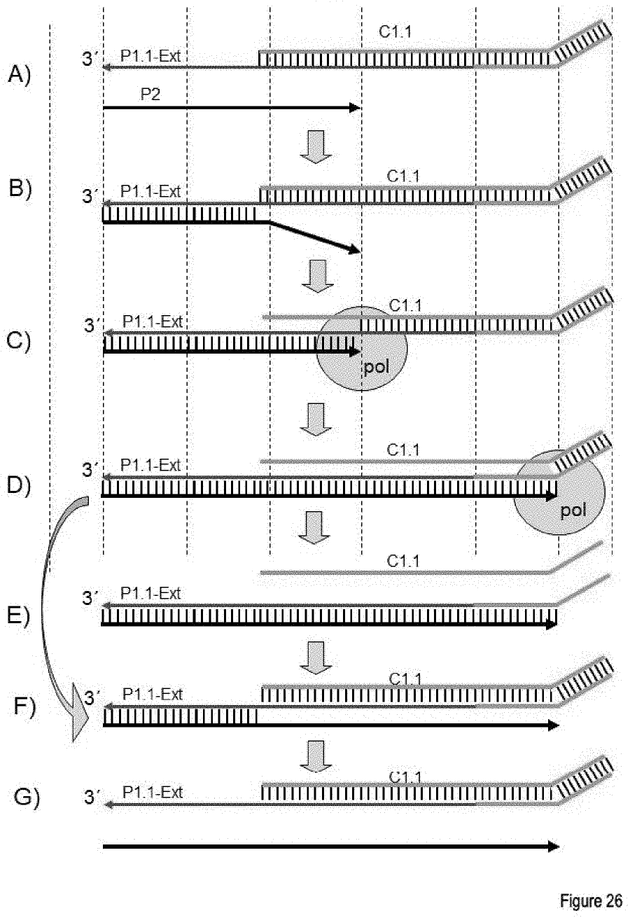

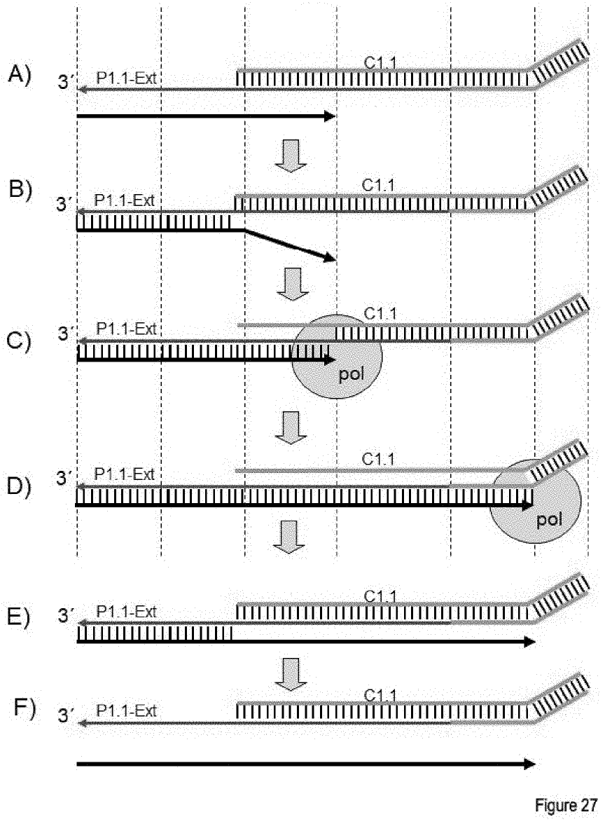

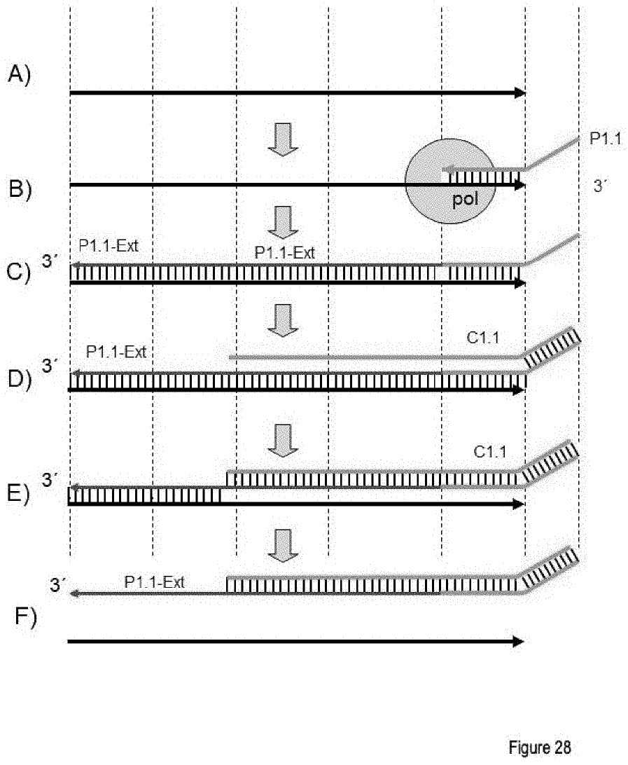

FIGS. 25 to 28 schematically show the interaction of components in an exponential amplification of a nucleic acid chain to be amplified in individual steps.

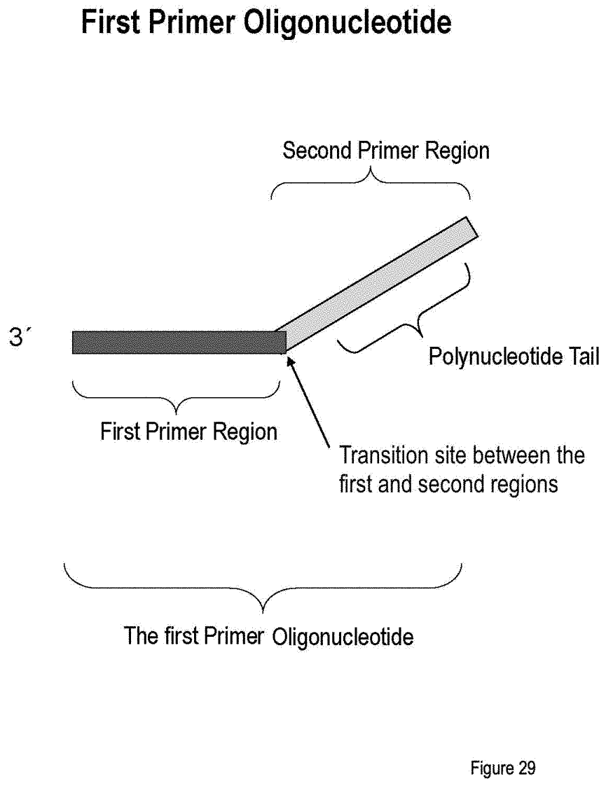

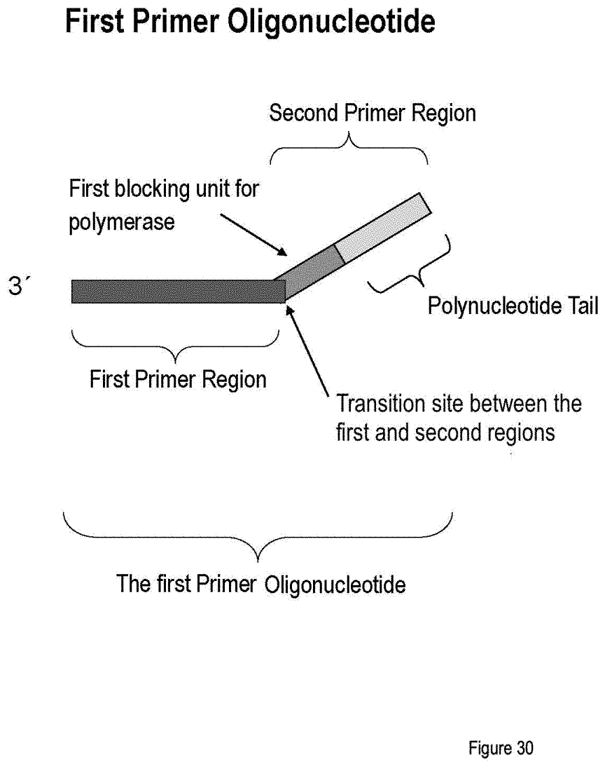

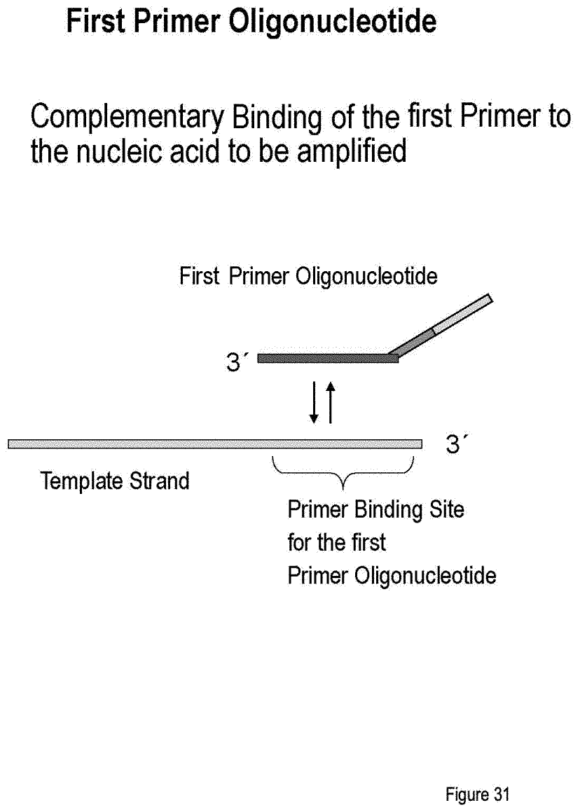

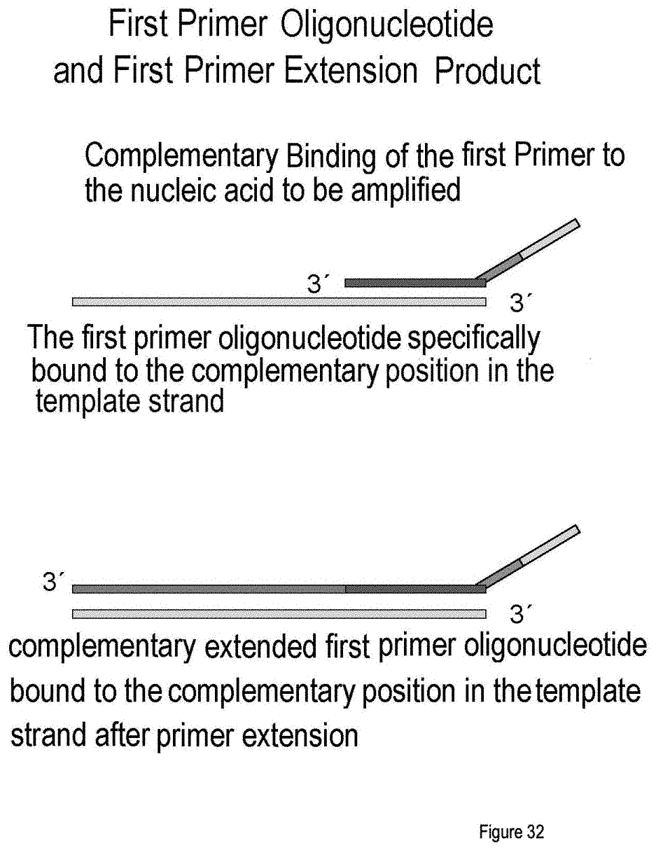

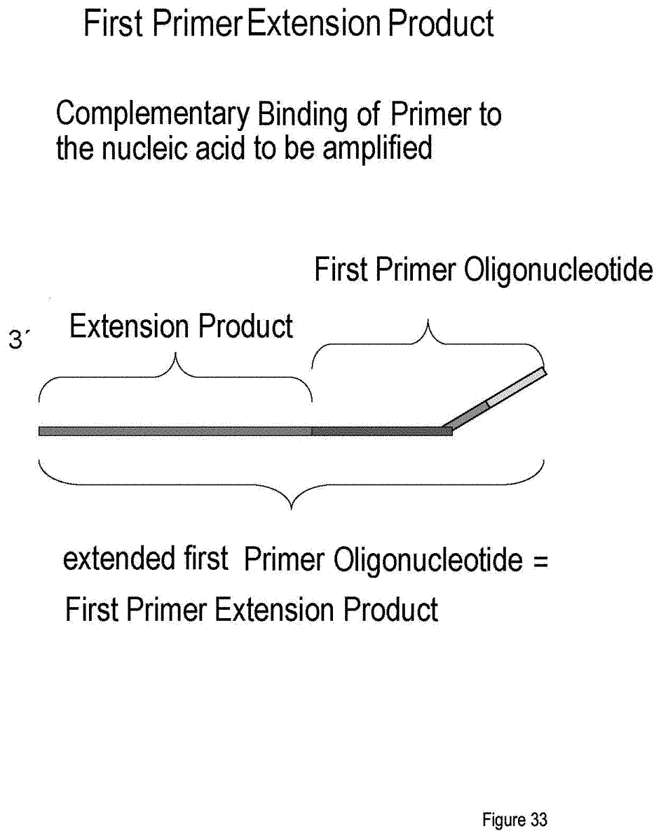

FIGS. 29-34 schematically show the structure of the first primer oligonucleotide and the activator oligonucleotide as well as the interaction between the first primer oligonucleotide and the template as well as the synthesis of the first primer extension product.

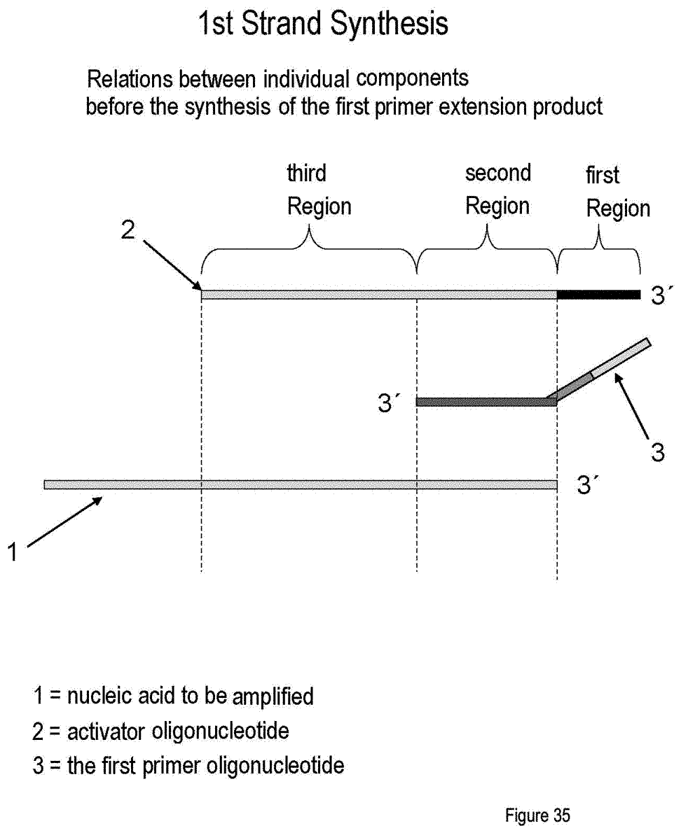

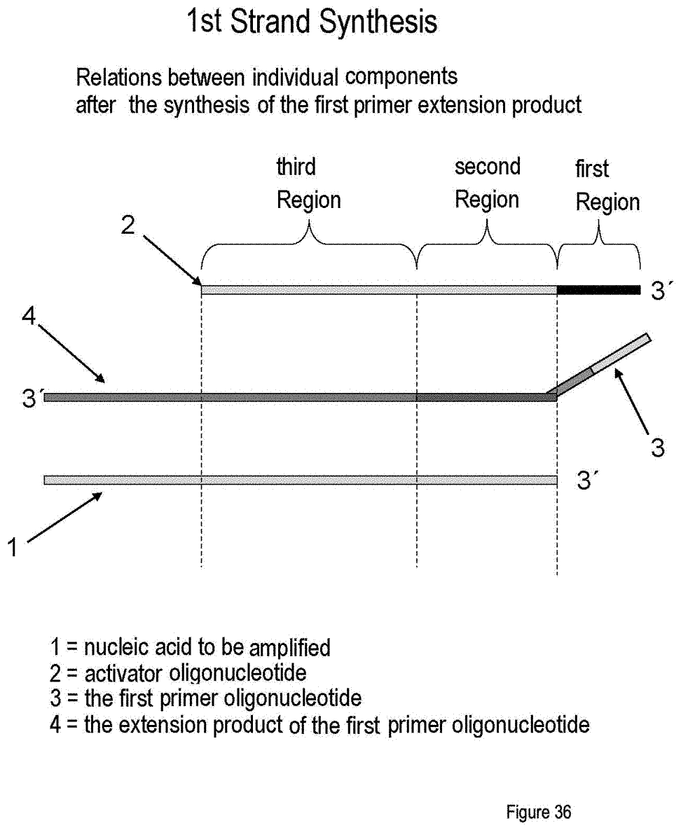

FIGS. 35-36 schematically show the interaction between structures during primer extension of the first primer oligonucleotide.

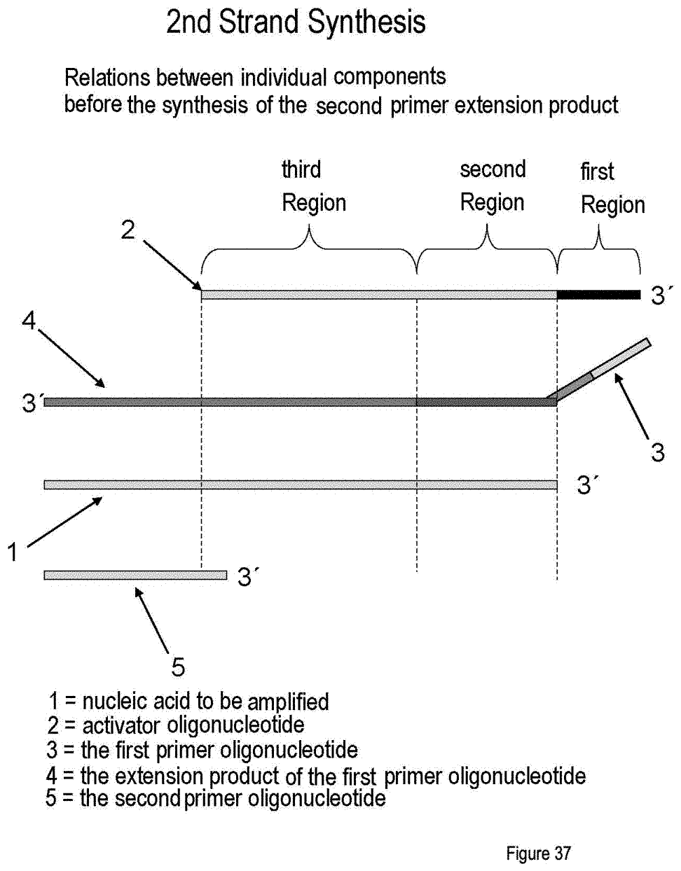

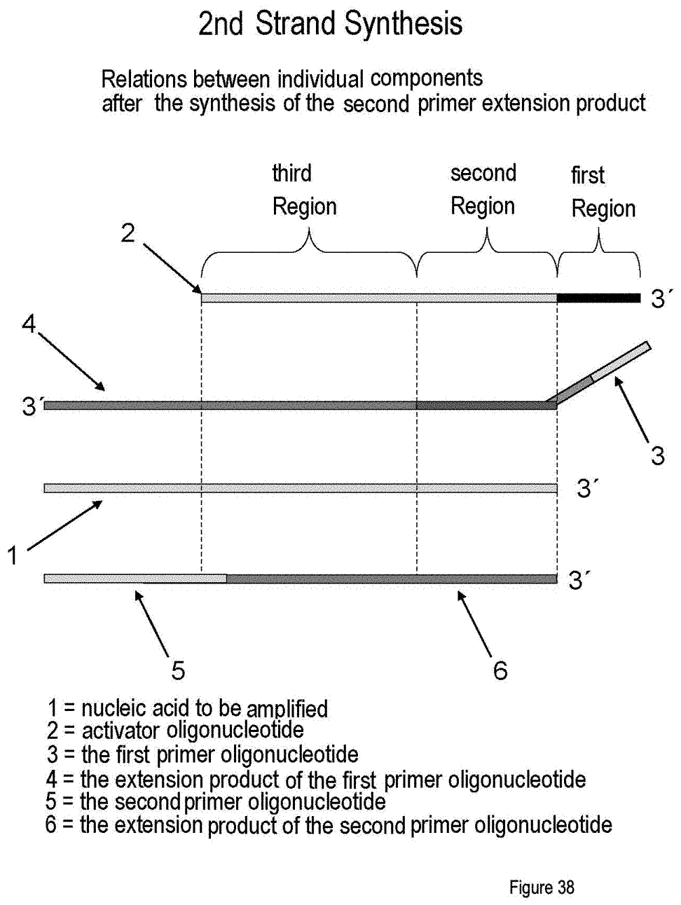

FIGS. 37-38 schematically show the interaction between structures during primer extension of the second primer oligonucleotide.

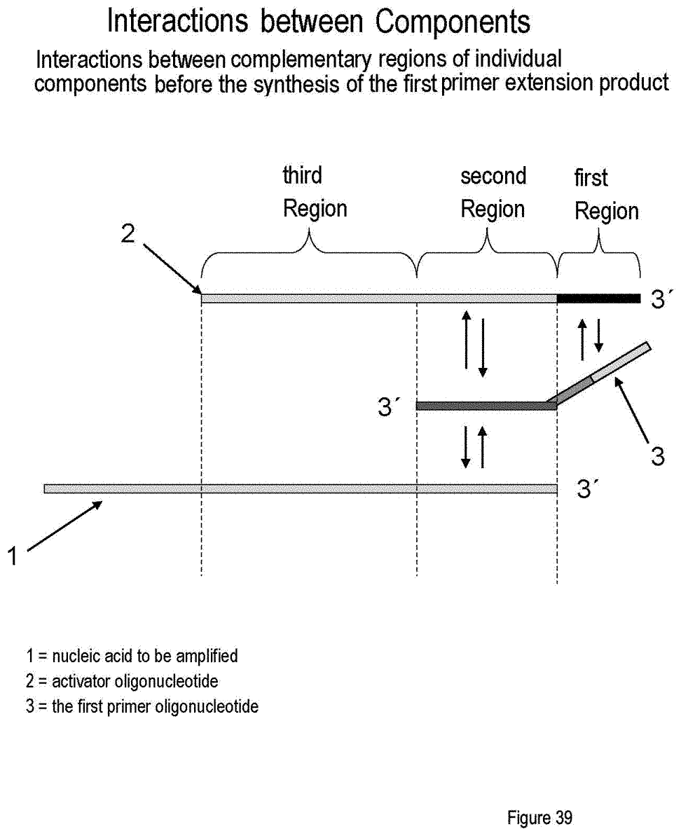

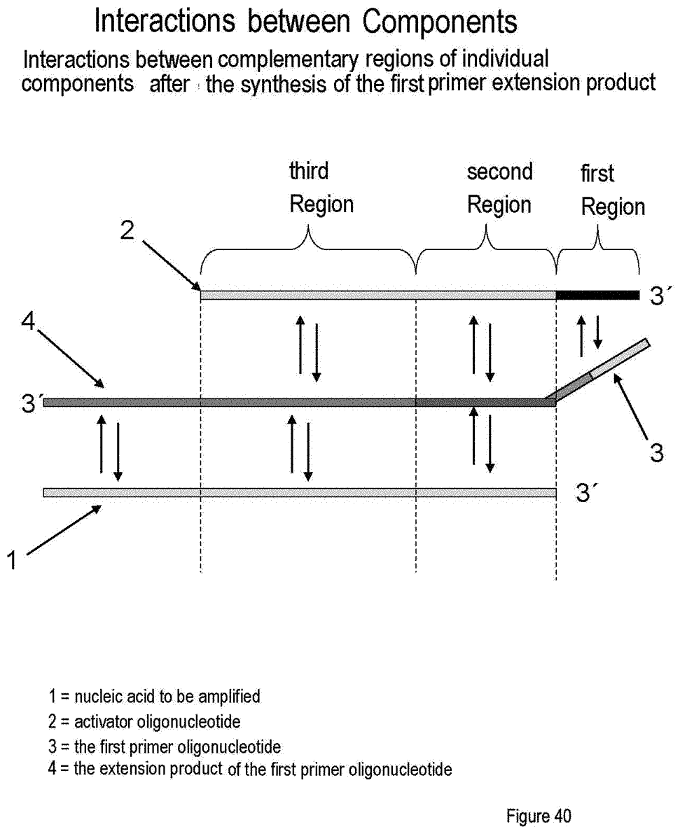

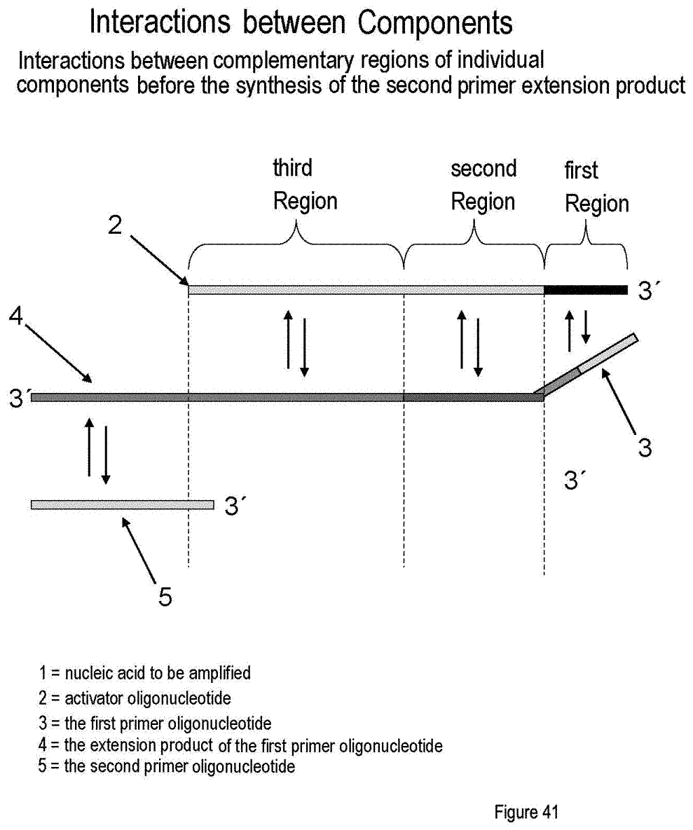

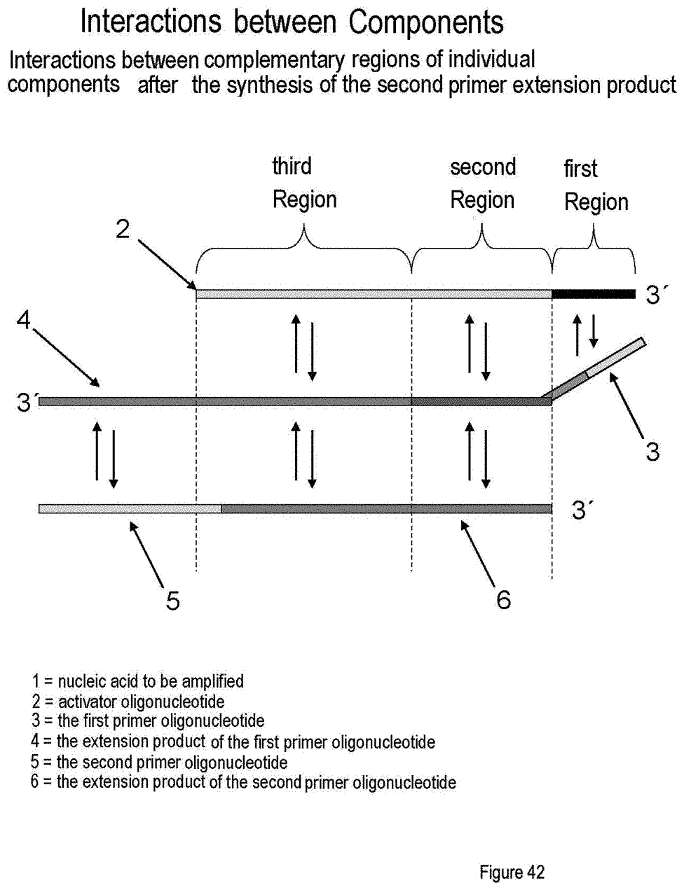

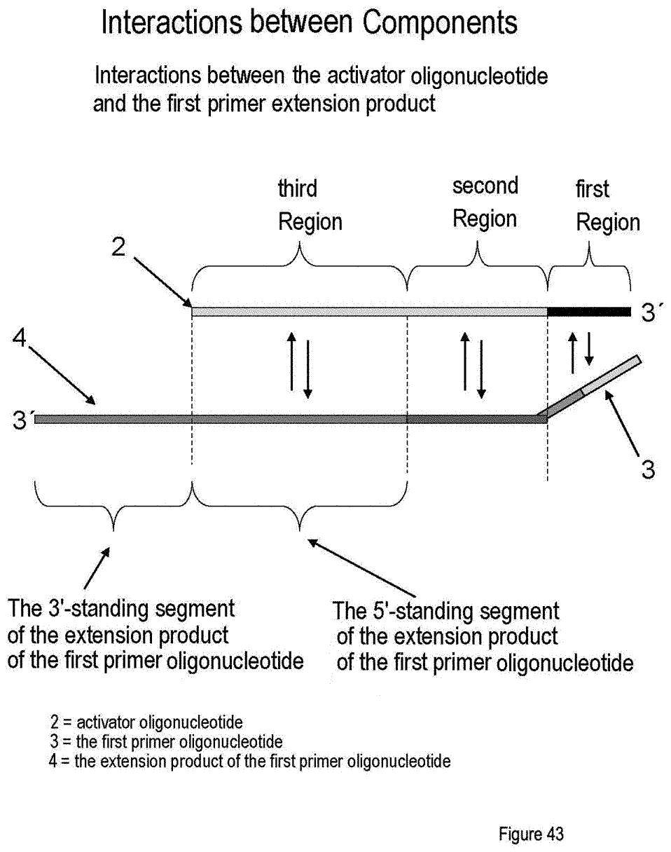

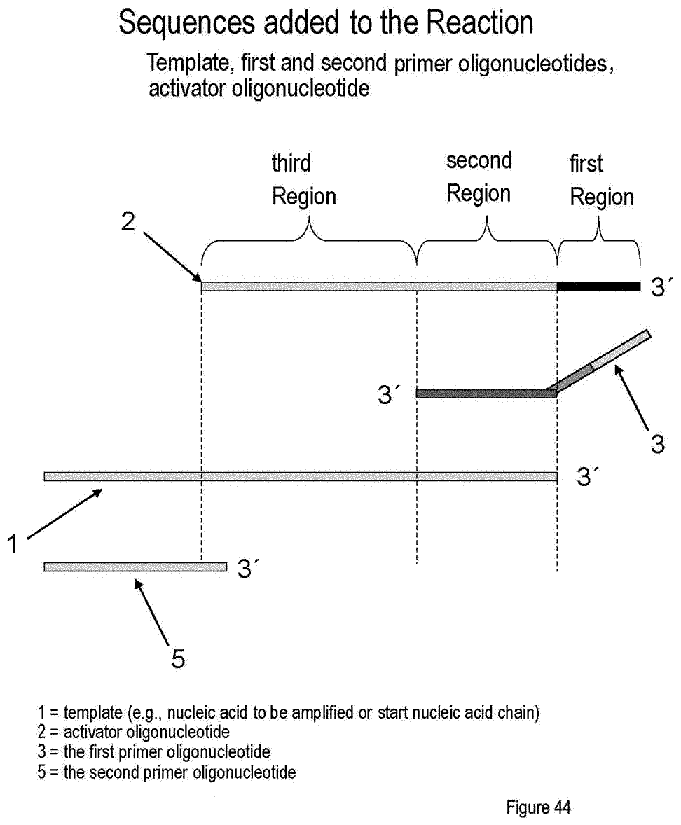

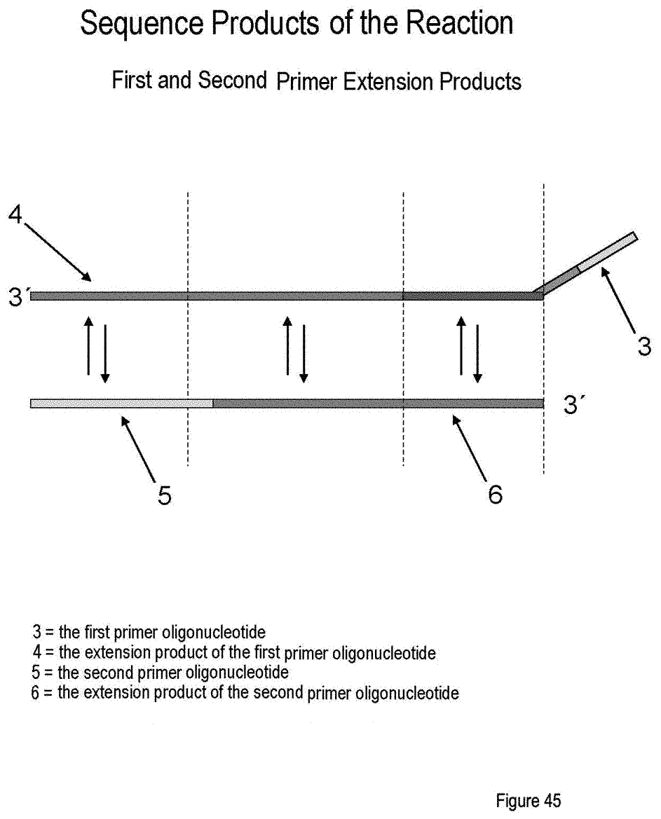

FIGS. 39-45 schematically show the interaction between single regions of components during amplification.

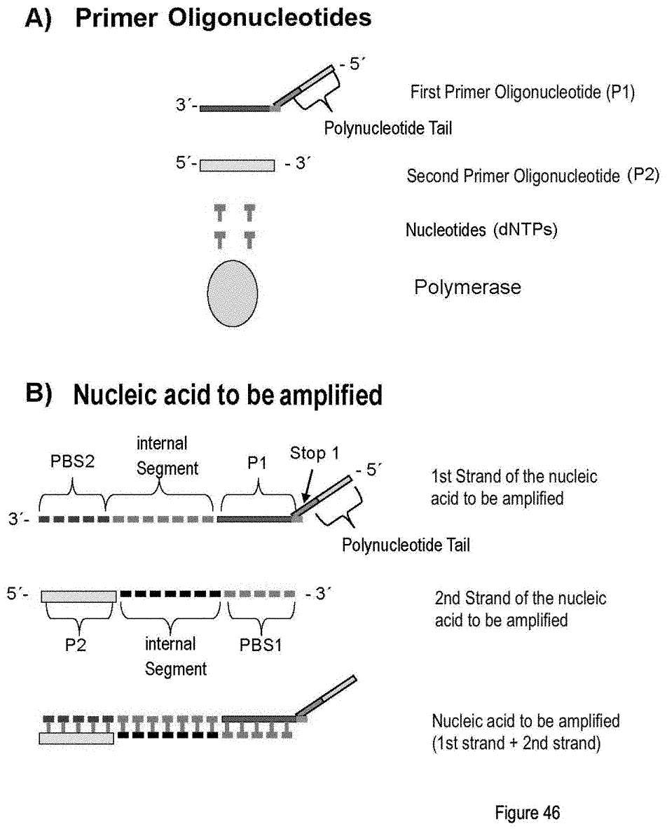

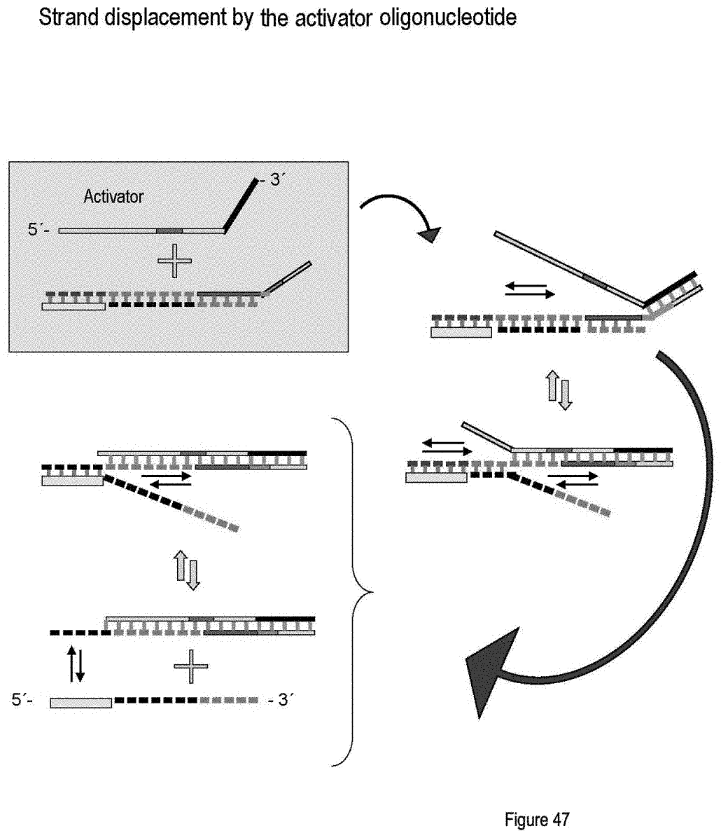

FIG. 46 describes the components of the structures illustrated in FIGS. 47-51 in more detail.

FIG. 47 schematically shows the strand displacement mechanism.

FIG. 48 schematically shows the interaction of the structures in strand displacement.

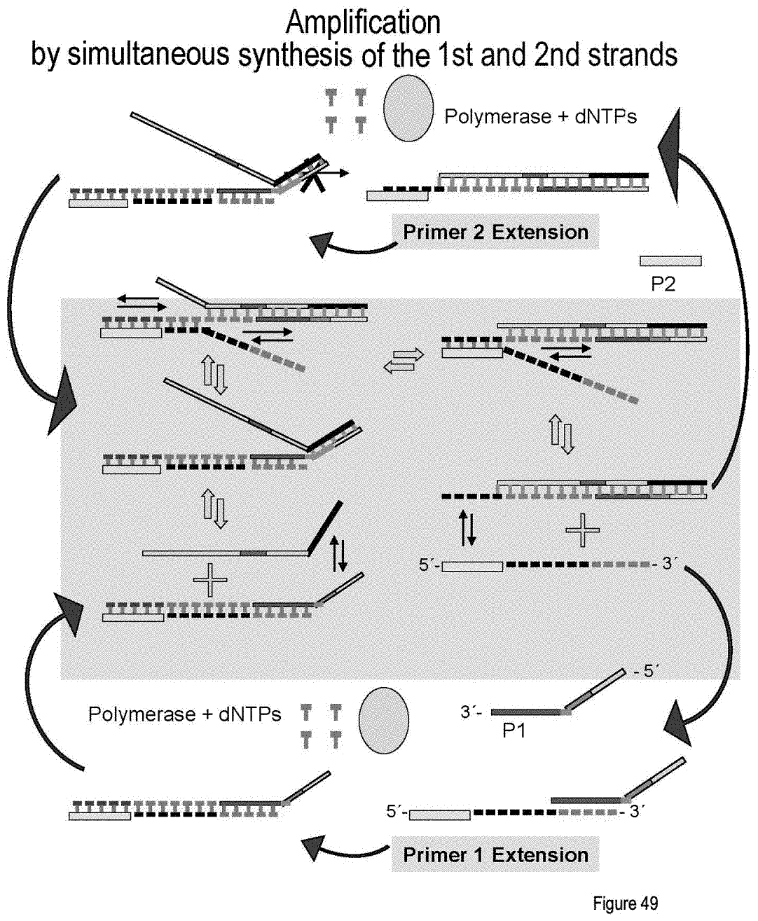

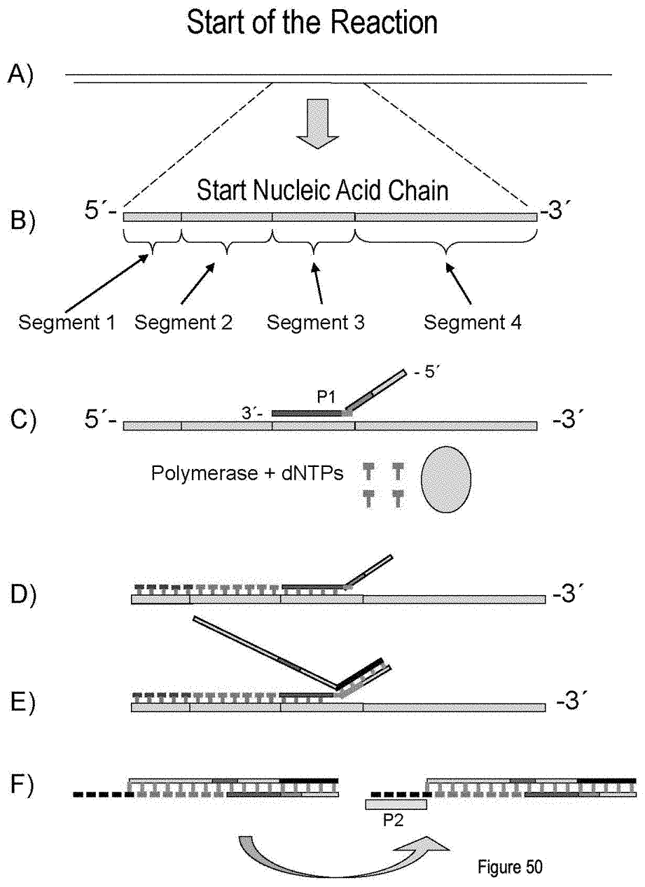

FIG. 49 schematically shows the interaction of the structures during nucleic acid amplification by amplification of the first primer oligonucleotide and the second primer oligonucleotide and further the effect of the activator oligonucleotide and the resulting strand displacement.

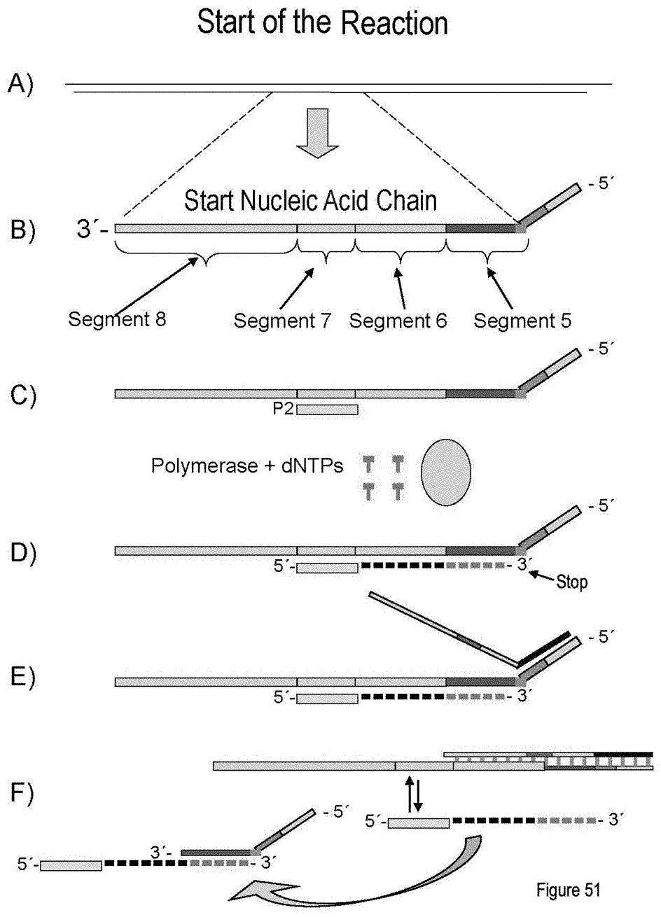

FIGS. 50-51 schematically show some embodiments of structures of a start nucleic acid chain and its use as a template at the beginning of the reaction.

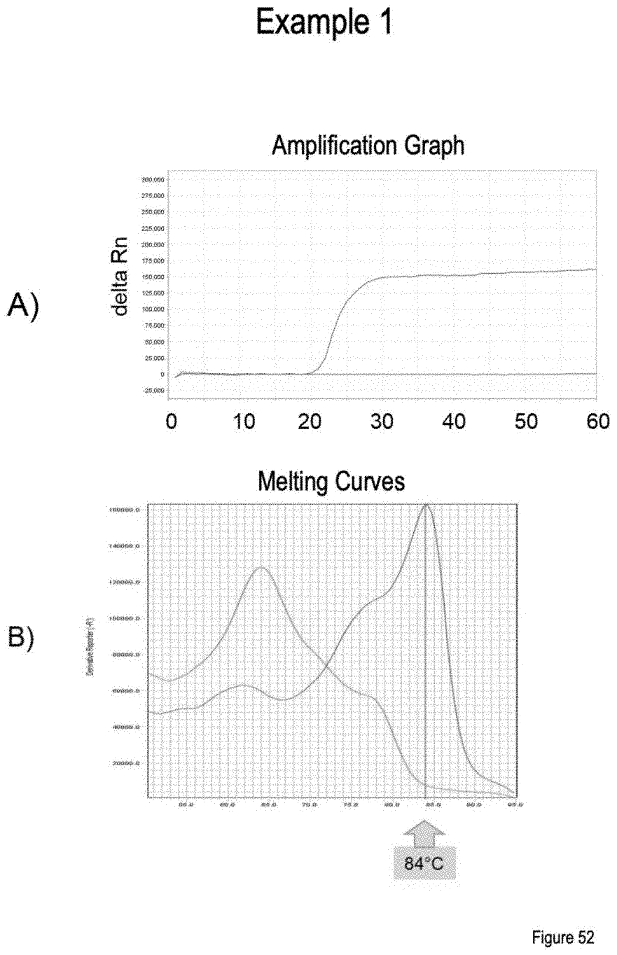

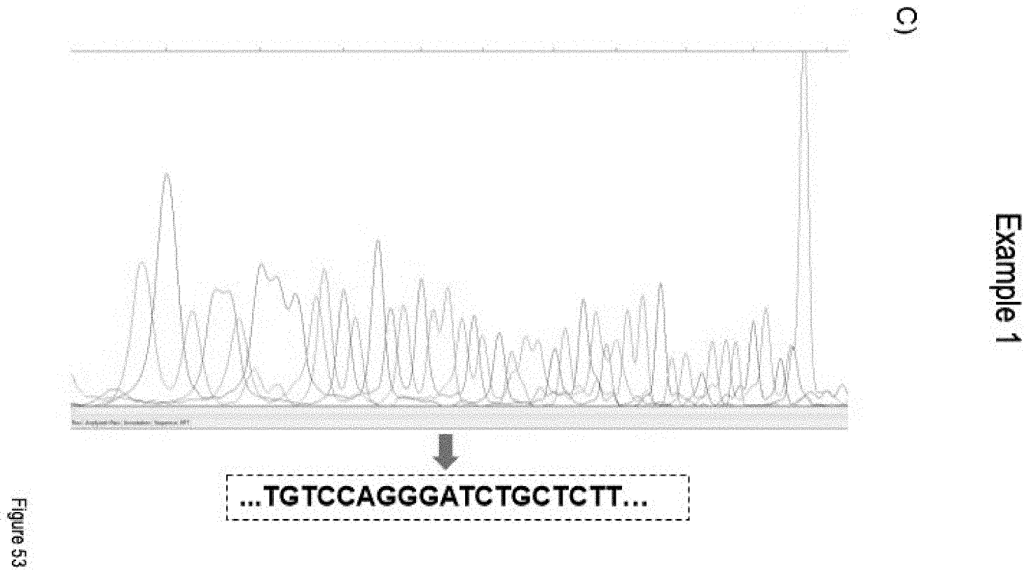

FIG. 52 shows results from example 1.

FIG. 53 shows results from example 2.

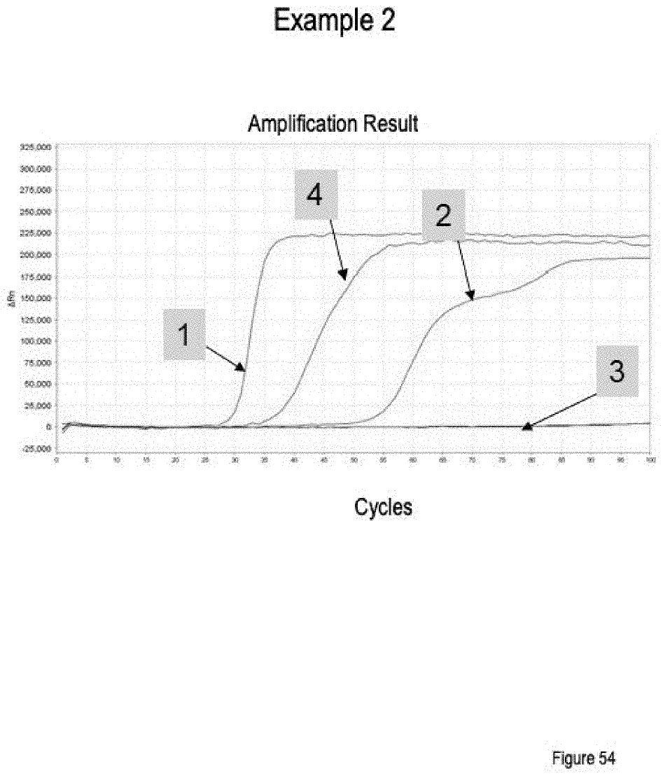

FIG. 54 shows results from example 3.

### DETAILED DESCRIPTION OF THE INVENTION

In the method according to the invention a single-stranded nucleic acid chain or a double-stranded nucleic acid that was converted into the single-stranded form can serve as a starting material. The amplification preferably is sequence-specific, i.e. preferably the nucleic acid to be amplified is multiplied.

A nucleic acid chain to be amplified, a first specific primer oligonucleotide, a second primer, and an activator oligonucleotide that takes part in the separation of the re-synthesized strands as well as a suitable polymerase and substrates such as dNTPs serve as components of the amplification system. The amplification takes place in a buffer solution under conditions that allow a primer extension reaction of both primers as well as support a strand displacement by the activator oligonucleotide for the separation of both primer extension products.

In one embodiment, all the method steps are performed under conditions that do not allow a separation of synthesized primer extension products in the absence of a suitable activator oligonucleotide. For example, the temperature of the reaction solution is selected such that the Tm of a double strand of both primer extension products is significantly above the reaction temperature.

Under these conditions a separation of both primer extension products takes places depending on the effect of the activator oligonucleotide. Said activator oligonucleotide is able to complementary bind to the first primer extension product and thereby displace the second primer extension product from its binding with the first primer extension product. In order to initiate said strand displacement reaction the first primer oligonucleotide is provided with a polynucleotide tail in its second region that can transiently bind to the activator oligonucleotide under reaction conditions and thus, causes a spatial proximity to other regions of the first primer extension nucleotide. After the initiation of the strand displacement by the activator oligonucleotide the second primer extension product is displaced from its binding with the first primer extension product. So, its 3′-standing segment becomes free and is available for further binding of a first primer oligonucleotide.

The polynucleotide tail of the first primer oligonucleotide preferably cannot be copied by a polymerase. This can be achieved either by using appropriate modifications in this region or by inserting a first blocking unit between the first primer region and the second primer region of the first primer oligonucleotide.

The synthesis of the second primer extension product takes place after the second primer oligonucleotide has been bound to the first primer extension product in its 3′-standing segment. Said segment preferably does not bind to the activator oligonucleotide and is sufficiently long to bind the second primer oligonucleotide and support a successful primer extension reaction. The synthesis of the second primer extension product takes place by displacing the activator oligonucleotide from the binding with the first primer extension product. For example, this can be done by polymerase-dependent strand displacement or also by strand displacement by means of the second primer.

Both primer extension products include copyable regions and mutually serve as a template. The activator oligonucleotide does not serve as a template. This can preferably be achieved by the use of nucleotide modifications that certainly can complementary bind to the first primer extension product, but are not accepted as a template by the polymerase. Examples of such nucleotide modifications are nucleotide compounds having modified phosphate sugar backbone portions, e.g., 2′-O-Alkyl-RNA modifications (e.g., 2′-OMe), LNA modifications, or morpholino modifications. In general, the presence of such modifications in a strand prevents a DNA-dependent polymerase from reading such a strand. The number of such modifications can be different, generally a few modifications (between 1 and 20) may be sufficient in order to prevent a polymerase from reading such a strand. Such nucleotide modifications can for example be used at or around the site of binding of the first primer oligonucleotide to the activator oligonucleotide and/or as constituents of the second region of the first primer oligonucleotide.

Owing to the use of such modifications the polymerase function is locally hindered, so that certain segments of the structures used cannot be copied by the polymerase and mainly remain single-stranded. In this single-stranded form they can further bind reaction components and thus, exercise their function.

Under reaction conditions that do not denature a double strand the use of the sequence-dependent nucleic acid-mediated strand displacement results in the sequence-specific separation of both primer extension products during the amplification reaction in the described method: a sufficient complementarity between re-synthesized extension fragments of the primer oligonucleotides with the sequence of an activator oligonucleotide given at the beginning of an amplification is a prerequisite for a successful strand displacement and thus, can have influence on the efficacy of the strand separation of a double strand (consisting of the first and second primer extension products). In case of minor divergences strand displacement and thus, also strand separation are decelerated. This can cause a deceleration of the entire reaction. With an increase in the difference in the sequence of the re-synthesized extension products and the sequence of the activator oligonucleotide given at the beginning of the reaction there is an increasing disability of the strand displacement that ultimately is no longer able to bring about a sufficient separation of both primer extension products. Both re-synthesized strands can no longer sufficiently be separated from each other, so that their binding sites are no longer accessible for primer oligonucleotides. In general, this leads to the termination of an amplification of sequences having sequence divergences.

In summary, not only the specificities of the binding of both primers with their templates, but also the nature of the sequence segments between the primers can have influence on the amplification, namely in that these sections allow a sufficient strand displacement or not, in accordance with their matching in the sequence of the activator oligonucleotide given at the beginning of the reaction. Thereby, the described method possibly overall can have a higher specificity than the conventional amplification methods.

The specific amplification further results by using the components at reaction conditions that preferably do not allow a spontaneous separation of re-synthesized primer extension products.

The method comprises several processes that are described below. These processes can be performed in one batch or in separate batches. If the processes are to be performed in one batch, so they can be performed under the same conditions, e.g., isothermal, or under different conditions, e.g., in thermocycling. Preferably, primer oligonucleotides and the activator oligonucleotide are present at the beginning of the reaction. However, a sequential addition of individual reagents is also possible.

Also, combinations with other amplification methods are possible, e.g., with PCR, wherein the PCR for example first is performed over 1 to 10 cycles and subsequently, it is for example went on working under isothermal conditions.

Essential Aspects of the Invention

### Embodiment 1

In an advantageous form the method for selective amplification of nucleic acid chains comprises the following steps:
1. Providing a nucleic acid chain to be amplified (a start nucleic acid chain) that comprises a target sequence, wherein said target sequence comprises at least one sequence segment (referred to as polymorphous locus) that can comprise at least two characteristic and different sequences (referred to as allele variants), further the target sequence comprises at least one further target sequence segment that is no polymorphous locus and is uniform and characteristic for said target sequence;
2. Providing at least one amplification system, each amplification system comprising:
   a. at least one first primer oligonucleotide
   b. at least one second primer oligonucleotide
   c. at least one polymerase
   d. at least one activator oligonucleotide
   wherein at least the activator oligonucleotide of an amplification system comprises a sequence composition that is specific and characteristic for the respective amplification system and enables the amplification of a characteristic and specific allele variant of a target sequence;
3. Realization of an amplification of a nucleic acid chain to be amplified, which comprises the following steps:
a) hybridizing a first primer oligonucleotide to the 3′ segment of a strand of a nucleic acid chain to be amplified, wherein the nucleic acid chain to be amplified comprises a target sequence with at least one polymorphous locus, wherein the first primer oligonucleotide comprises:
   a first primer region in the 3′ segment of the first primer oligonucleotide that can sequence-specifically bind to a strand of a nucleic acid chain to be amplified,
   a second region that is directly or via a linker linked to the 5′ end of the first primer region of the first primer oligonucleotide and that comprises a polynucleotide tail which is suitable for binding an activator oligonucleotide and supporting strand displacement (step c) by the activator oligonucleotide, wherein the polynucleotide tail remains substantially uncopied by polymerase under the selected reaction conditions,
b) template-dependent extension of the first primer oligonucleotide using a nucleic acid chain as a template strand (comprising either a nucleic acid chain to be amplified and/or a start nucleic acid chain and/or a second primer extension product) with the help of a template-dependent polymerase to form a first primer extension product comprising sequence parts complementary to the target sequence and/or to the nucleic acid chain to be amplified (a),
c) binding the activator oligonucleotide to the polynucleotide tail of the second region of the first extended primer oligonucleotide, wherein the activator oligonucleotide comprises:
   a first single-stranded region that can bind to the polynucleotide tail of the second region of the first primer oligonucleotide,
   a second single-stranded region that is complementary and can bind to the first region of the first primer oligonucleotide,
   a third single-stranded region that is substantially complementary to at least a segment of the extension product, which has been synthesized by polymerase, of the first primer extension product,
   wherein the activator oligonucleotide does not serve as template for a primer extension of the first primer oligonucleotide,
d) binding the activator oligonucleotide to the first primer region of the first extended primer oligonucleotide by displacing the strand of the nucleic acid chain to be amplified that is complementary to said first primer region,
e) binding the activator oligonucleotide to the complementary segment of the extension product of the first extended primer oligonucleotide by displacing the strand of the nucleic acid chain to be amplified that is complementary to said extension product, wherein the 3′ segment of the first primer extension product becomes single-stranded,
f) hybridizing a second oligonucleotide primer to the first primer extension product, wherein the 3′ segment of the second oligonucleotide primer comprises a sequence that can hybridize to the first primer extension product; and
g) template-dependent extension of the second oligonucleotide primer using the first primer extension product as a template with a template-dependent polymerase to form a second primer extension product, wherein extension is up to and including the first primer region of the first primer oligonucleotide and said first primer region is copied by the polymerase, wherein the polynucleotide tail of the second region remains uncopied,
h) repeating steps a)-g) until the desired degree of amplification has been achieved.

A preferred embodiment according to aspect 3 is characterized in that at least one of the components first primer, activator oligonucleotide, and second primer has a variation to a sequence variant of a polymorphous locus of the target sequence of at least one nucleotide to the perfect-match sequence of the target sequence, so that there is no perfect attachment of this component to the target sequence.

A preferred embodiment according to aspect 3 is characterized in that at least two variants of at least one of the components first primer oligonucleotide, activator oligonucleotide, and second primer oligonucleotide are present, wherein each of said variants differs from the other variants by at least one nucleotide.

A preferred embodiment according to aspect 3 is characterized in that at least two variants of at least two of the components selected from first primer oligonucleotide, activator oligonucleotide, and second primer oligonucleotide are present, wherein each of said variants differs from the target sequence to which the variants are substantially complementary by at least one oligonucleotide.

A preferred embodiment according to aspect 3 is characterized in that the target sequence comprises at least one polymorphous locus, wherein each polymorphous locus comprises at least two sequence variants of the target sequence.

A preferred embodiment according to aspect 3 is characterized in that at least two variants of a target sequence comprising at least one polymorphous locus are present in the reaction batch at the same time.

A preferred embodiment according to aspect 3 is characterized in that at least two variants of a target sequence are present in the reaction batch at the same time, wherein preferably one of the variants of the target sequence is amplified.

A preferred embodiment according to aspect 3 is characterized in that at least two variants of a target sequence are present in the reaction batch at the same time, wherein one of the variants of the target sequence is preferably not amplified.

A preferred embodiment according to aspect 3 is characterized in that at least two variants of a target sequence are present in the reaction batch at the same time, wherein preferably one of the variants of the target sequence is amplified and at the same time at least one of the other variants of the target sequence is preferably not amplified.

In one embodiment a target sequence comprises a polymorphous locus that comprises at least one nucleotide position that comprises at least two characteristic sequence variants in one target sequence.

In a further embodiment a target sequence comprises a polymorphous locus that comprises at least two nucleotide positions that comprise at least two characteristic sequence variants in one target sequence.

In one embodiment a target sequence comprises a polymorphous locus that comprises a length between one nucleotide and 100 nucleotides, wherein such a locus comprises at least two characteristic sequence variants of a target sequence.

In one embodiment in step (1) at least two start nucleic acid chains are provided, wherein each start nucleic acid chain comprises a variant of a target sequence having a specific and characteristic sequence variant of a polymorphous locus.

In a further embodiment in step (1) at least two start nucleic acid chains are provided, wherein each start nucleic acid chain comprises an own target sequence and said target sequences of the first and of one further start nucleic acid chain are different.

### Embodiment 2

Method according to embodiment 1 that in step 2 comprises at least one amplification system comprising a specific activator oligonucleotide that comprises a sequence segment corresponding to the polymorphous locus that can form a complementary perfect-match bond with a characteristic and specific sequence variant of the polymorphous locus of a nucleic acid chain to be amplified provided in step 1 and comprising a target nucleic acid, or with its complementary strand.

### Embodiment 3

Method according to embodiment 2 that in step 2 comprises at least one amplification system comprising a specific activator oligonucleotide that comprises a characteristic and specific complementary sequence segment corresponding to the polymorphous locus, and further comprises at least one further sequence segment that can substantially complementary bind to at least one specific segment of a target sequence, wherein said segment of the target sequence is not a polymorphous locus.

In one embodiment the amplification system provided in step 2 comprises
an activator oligonucleotide as defined in embodiment 2 or 3 and
a first primer oligonucleotide that can substantially complementary bind to the sequence fragment that is uniform for the provided target sequence and
a second primer oligonucleotide that can substantially complementary bind to the sequence fragment that is uniform for the provided target sequence
wherein the first primer oligonucleotide and the second primer oligonucleotide comprise no sequence segments that are specific for a sequence variant of the target sequence. Rather, the first primer oligonucleotide and the second primer oligonucleotide can completely complementary or substantially complementary bind to the target sequence, without differentiating the sequence variants.
Thus, such an amplification system is constructed such that the polymorphous locus is located in the sequence segment that is between both primers. Thus, the target sequence comprises one target sequence-specific sequence segment each on both sides of a polymorphous locus.
Thus, the segment of the activator oligonucleotide corresponding to the polymorphous locus is in the third region of the activator oligonucleotide.

In a further embodiment the amplification system provided in step 2 comprises
an activator oligonucleotide as defined in embodiment 2 or 3 and
a first sequence variant-specific primer oligonucleotide that at least partially (e.g., with its 3′-terminal nucleotide) can fully complementary bind to the specific sequence segment of a polymorphous locus of a provided target sequence
a second primer oligonucleotide that binds to the sequence fragment that is uniform for the provided target sequence and
wherein the second primer oligonucleotide comprises no sequence segments that are characteristic and specific for only one sequence variant of the target sequence. Rather, the second primer oligonucleotide can completely complementary or substantially complementary bind to the target sequence, without differentiating the sequence variants.
Thus, in one embodiment such an amplification system is constructed such that the sequence of the first primer oligonucleotide at least with one nucleotide position overlaps with a characteristic sequence of the polymorphous locus.
In one embodiment the segment of the activator oligonucleotide corresponding to the polymorphous locus completely lies in the second region of the activator oligonucleotide.
In a further embodiment, the segment of the activator oligonucleotide corresponding to the polymorphous locus partially lies in the second and partially in the third regions of the activator oligonucleotide.

In a further embodiment the amplification system provided in step 2 comprises
an activator oligonucleotide as defined in embodiment 2 or 3 and
a first primer oligonucleotide that binds to the sequence fragment that is uniform for the provided target sequence; and
a second sequence variant-specific primer oligonucleotide that at least partially (e.g., with its 3′-terminal nucleotide) can fully complementary bind to the specific sequence segment of a polymorphous locus of a provided target sequence
wherein the first primer oligonucleotide comprises no sequence segments that are characteristic and specific for only one sequence variant of the target sequence. Rather, the first primer oligonucleotide can completely complementary or substantially complementary bind to the target sequence, without differentiating the sequence variants.
Thus, in one embodiment such an amplification system is constructed such that the sequence of the second primer oligonucleotide at least with one nucleotide position overlaps with a characteristic sequence of the polymorphous locus.
In one embodiment the segment of the activator oligonucleotide corresponding to the polymorphous locus completely lies in the third region of the activator oligonucleotide.

In a further embodiment the amplification system provided in step 2 comprises
an activator oligonucleotide as defined in embodiment 2 or 3 and
a first sequence variant-specific primer oligonucleotide that at least partially (e.g., with its 3′-terminal nucleotide) can fully complementary bind to the specific sequence segment of a polymorphous locus of a provided target sequence
a second sequence variant-specific primer oligonucleotide that at least partially (e.g., with its 3′-terminal nucleotide) can fully complementary bind to the specific sequence segment of a polymorphous locus of a provided target sequence.
Thus, in one embodiment such an amplification system is constructed such that the sequence of the first and second primer oligonucleotide at least with one nucleotide position overlaps with a characteristic sequence of the polymorphous locus.
In one embodiment the segment of the activator oligonucleotide corresponding to the polymorphous locus lies in the second and third region of the activator oligonucleotide. Here, the length of the polymorphous locus preferably comprises regions between 5 and 100 nucleotides.

### Embodiment 4

Method according to embodiment 1 in which only one start nucleic acid chain provided in step 1 and comprising a specific and characteristic sequence variant of a polymorphous locus is preferably to be amplified as a nucleic acid to be amplified that comprises a first specific and characteristic sequence in the polymorphous locus.

Method according to embodiment 4 in which only one start nucleic acid chain provided in step 1 and comprising a specific and characteristic sequence variant of a polymorphous locus is preferably to be amplified as a nucleic acid to be amplified that comprises a first specific and characteristic sequence in the polymorphous locus, wherein optionally at least one second specific start nucleic acid chain provided in step 1 and comprising a second characteristic sequence in the polymorphous locus in its amplification is less preferably to be amplified or its amplification is to be suppressed, respectively. Here, the first and second characteristic sequence variants are not identical.

### Embodiment 5

Method according to embodiment 4 which in step 1 comprises at least two start nucleic acid chains, wherein each start nucleic acid chain comprises a characteristic and specific sequence variant of a target nucleic acid, and

in step 2 only one specific amplification system is provided that comprises an activator oligonucleotide that is specific and characteristic for one sequence variant of the target sequence, so that in step 3 only one characteristic and specific nucleic acid chain to be amplified is preferably amplified, wherein the specific amplified nucleic acid chain can form a perfect-match bond with the sequence segment of an activator oligonucleotide used that is characteristic for said nucleic acid chain to be amplified and corresponds to the polymorphous locus.

### Embodiment 6

Method according to embodiment 4 which in step 1 comprises at least two start nucleic acid chains, wherein each start nucleic acid chain comprises a characteristic and specific sequence variant of a target nucleic acid, and

in step 2 only one specific amplification system is provided that comprises an activator oligonucleotide that is specific and characteristic for one sequence variant of the target sequence (as described in embodiment 5) and further at least one primer oligonucleotide specific for a sequence variant of a target sequence (the first primer oligonucleotide or the second primer oligonucleotide), so that in step 3 only one characteristic and specific nucleic acid chain to be amplified is preferably amplified, wherein the specific amplified nucleic acid chain can form a perfect-match bond with the sequence segment of an activator oligonucleotide used that is characteristic for said nucleic acid chain to be amplified and corresponds to the polymorphous locus.

### Embodiment 7

Method according to embodiment 6, wherein the 3′-terminal segment or the 3′-terminal nucleotide of a primer (a first primer or a second primer) can form a complementary perfect-match bond with a characteristic and specific sequence variant of the polymorphous locus of a start nucleic acid chain comprising a target sequence provided in step 1 or with its complementary strand and under reaction conditions, if complementary bound to a template strand comprising said sequence variant, can be extended by a polymerase, so that this results in a specific and characteristic primer extension product.

### Embodiment 8

Method according to any one of embodiments 1 to 7 that in step 2 comprises at least one amplification system that additionally comprises at least one competitor primer oligonucleotide that is not identical to the first or second primer of an amplification system; and

said competitor primer comprises at least one sequence segment corresponding to the polymorphous locus, which can form a complementary perfect-match bond with a characteristic and specific sequence variant of the polymorphous locus of a target nucleic acid provided in step 1 or with its complementary strand and under reaction conditions can be extended by a polymerase, so that this results in a specific and characteristic competitor primer extension product.

In one embodiment, the competitor oligonucleotide used can completely or partially bind to the segment of the target sequence that can form a complementary bond with the first region of the first primer oligonucleotide.

In one embodiment the competitor oligonucleotide used can completely or partially bind to the segment of the target sequence that can form a complementary bond with the second region of the first primer oligonucleotide.

In one embodiment said competitor primer oligonucleotide is fully complementary to a sequence variant of a target sequence that is not identical to the sequence variant of the nucleic acid chain to be amplified.

In a further embodiment amplification of the sequence variant which can form perfect match to the competitor primer oligonucleotide is selectively suppressed using the competitor oligonucleotide.

In one embodiment of the method a competitor primer oligonucleotide comprises no sequence segment which can substantially complementary bind with the first region of the activator oligonucleotide.

### Embodiment 9

Method according to embodiment 1 in which at least two nucleic acid chains to be amplified provided in step 1 are to be amplified, wherein

a first nucleic acid chain to be amplified comprises a first specific and characteristic sequence in the polymorphous locus and each further nucleic acid chain to be amplified comprises a sequence in the polymorphous locus that is specific and characteristic for it, and

in step 2 for each nucleic acid chain to be amplified a specific amplification system is provided in which at least the activator oligonucleotide comprises a segment that is specific for each nucleic acid chain to be amplified and corresponds to the polymorphous locus,

in step 3 specific amplification of each nucleic acid chain to be amplified takes place with the help of a corresponding specific activator oligonucleotide.

### Embodiment 10

Method according to embodiment 9 in which for each specific and characteristic variant of a target sequence provided in step 1 in step 2 at least two specific amplification systems are provided, wherein each specific amplification system comprises a specific activator oligonucleotide for one of the provided nucleic acid chains to be amplified comprising a specific and characteristic sequence variant of a target nucleic acid chain.

### Embodiment 11

Method according to embodiment 1 in which at least two different target sequences are provided and in step 2 at least two amplification systems each specific for individual target sequences are provided, wherein each specific amplification system comprises one target sequence-specific activator oligonucleotide for one of the provided nucleic acid chains to be amplified each and two target sequence-specific primers for one of the provided nucleic acid chains to be amplified each comprising a specific and characteristic target sequence. As a result, different target sequences are amplified with different amplification systems each.

In one embodiment, the method is performed under conditions that do not allow a separation of complementary strands of the nucleic acid to be amplified in the absence of activator oligonucleotide.

In one embodiment, copying the polynucleotide tail is caused in the second primer region by a stopping region for the polymerase that is arranged between the first and the second regions.

In one embodiment, the third single-stranded region of the activator oligonucleotide is substantially complementary to the segment of the extension product, which has been synthesized by the polymerase, of the first primer extension product, which immediately follows the first primer region, wherein:

In one embodiment, the third single-stranded region of the activator oligonucleotide is completely complementary to the mentioned 5′ segment of the extension product of the first primer extension product, wherein the length of said complementary sequence part comprises the following ranges: of at least 3 to 70 nucleotides, better of at least 5 to 50 nucleotides, preferably of 5 to 40 nucleotides, further preferably of 5 to 30 nucleotides, particularly preferred of 5 to 20 nucleotides. Particularly preferred is the embodiment in which the completely complementary segment lies correspondingly to the polymorphous locus.

In a further embodiment, the sequences of the third single-stranded region of the activator oligonucleotide and the corresponding sequence of the mentioned 5′ segment of the extension product of the first primer extension product comprise complementary sequences except for one sequence position (a pair of nucleotides/bases) having a non-complementary base pairing (in the meaning of Watson-Crick base pairing) over a length of at least 3 to 70 nucleotides, better of at least 5 to 60 nucleotides, preferably of 10 to 40 nucleotides, particularly preferred of 10 to 20 nucleotides. Thus, binding of the activator oligonucleotide is substantially complementary. Preferably, the sequence position with non-complementary base pairs lies outside the sequence segment that corresponds to the polymorphous locus.

In a further embodiment, the sequences of the third single-stranded region of the activator oligonucleotide and the corresponding sequence of the mentioned 5′ segment of the extension product of the first primer extension product comprise complementary sequences except for two sequence positions (a pair of nucleotides/bases) having a non-complementary base pairing (in the meaning of Watson-Crick base pairing) over a length of at least 3 to 70 nucleotides, better of at least 5 to 60 nucleotides, preferably of 10 to 40 nucleotides, particularly preferred of 10 to 20 nucleotides. Thus, binding of the activator oligonucleotide is substantially complementary. Preferably, the sequence position with non-complementary base pairs lies outside the sequence segment that corresponds to the polymorphous locus.

In a further embodiment, the sequences of the third single-stranded region of the activator oligonucleotide and the corresponding sequence of the mentioned 5′ segment of the extension product of the first primer extension product comprise complementary sequences (in the meaning of Watson-Crick base pairing) over a length of at least 3 to 70 nucleotides, better of at least 5 to 60 nucleotides, preferably of 10 to 40 nucleotides, particularly preferred of 10 to 20 nucleotides, further said segments comprise non-complementary regions in at least three sequence positions, wherein said positions are within the 5′ segment of the third section of the activator oligonucleotide.

Thus, binding of the activator oligonucleotide is substantially complementary. Preferably, the sequence position with non-complementary base pairs lies outside the sequence segment that corresponds to the polymorphous locus.

In a further embodiment, the sequences of the third single-stranded region of the activator oligonucleotide and the sequence of the mentioned 5′ segment of the extension product of the first primer extension product comprise complementary sequences except for at least one and at most ten sequence positions having a non-complementary base pairing (in the meaning of Watson-Crick base pairing) over a length of at least 3 to 70 nucleotides, better of at least 5 to 60 nucleotides, preferably of 10 to 40 nucleotides, particularly preferred of 10 to 20 nucleotides, wherein in sequence positions having non-complementary base pairing (in the meaning of Watson-Crick base pairing) at least one modified nucleotide having modified nucleobases is involved. Such modified nucleobases comprise for example nucleobases with enhanced binding of natural nucleobases (e.g., 2-amino adenines), or with attenuated binding such as for example so-called universal bases such as inosines or 5-nitroindole. The modified nucleobases are preferably located in the third sequence region of the activator oligonucleotide. Thus, binding of the activator oligonucleotide is substantially complementary. Preferably, the sequence position with non-complementary base pairs lies outside the sequence segment that corresponds to the polymorphous locus.

In a further embodiment of the method step (e) of the method is further modified and comprises:

the binding of the activator oligonucleotide to the complementary segment of the extension product of the first extended primer oligonucleotide by displacing the strand of the nucleic acid chain to be amplified that is complementary to said extension product until said complementary strand of the nucleic acid to be amplified is detached from the first primer extension product, wherein the 3′ segment of the first primer extension product becomes single-stranded.

In a further embodiment of the method step (f) of the method is further modified and comprises:

the hybridization of a second oligonucleotide primer to the first primer extension product, wherein at the same time there is at least a partial displacement of the activator oligonucleotide from the binding with the first extension product by strand displacement.

In a further embodiment of the method step (g) of the method is further modified and comprises a displacement of the activator oligonucleotide from the binding with the first primer extension product with the participation of the polymerase.

In a further embodiment of the method step (h) of the method is further modified and comprises: optionally the binding of the activator oligonucleotide to the uncopied polynucleotide tail of the first extended primer oligonucleotide and a displacement of the second primer extension product from the binding to the first primer extension product with the simultaneous formation of a complementary double strand with a segment of the first specific extension product of the first primer oligonucleotide.

In a further embodiment of the method the method is further modified and comprises: h) continuation of the reaction under conditions that allow a repletion of steps (a) to (g).

In a further embodiment of the method the method is further modified and comprises: the simultaneous amplification of the first and second primer extension products in an exponential reaction by using the first and second primer oligonucleotides and the activator oligonucleotide, wherein the formed primer extension products function as a template for the mutual synthesis.

Reaction Conditions

The reaction conditions comprise among others buffer conditions, temperature conditions, duration of the reaction, and concentrations of respective reaction components.

During the reaction the amount of the specifically produced nucleic acid to be amplified accumulates in an exponential manner. The reaction comprising the synthesis of the extension products can be carried out for the production of the desired amount of the specific nucleic acid sequence as long as needed. The method according to the invention is preferably carried out continuously. In a preferable embodiment, the amplification reaction proceeds at the same reaction temperature, wherein the temperature is preferably between 50° C. and 70° C. In another embodiment, the reaction temperature can also be controlled variably, so that single steps of the amplification each proceed at different temperatures.

The reagents needed for the exponential amplification are preferably present already at the beginning of a reaction in the same batch. In another embodiment, reagents can also be added in later stages of the method.

Preferably, no helicases or recombinases are used in the reaction mixture for the separation of the newly synthesized double strands of the nucleic acid to be amplified.

In a preferred embodiment, the reaction mixture does not contain biochemical energizing compounds such as ATP.

The amount of the nucleic acid to be amplified that is present at the beginning of the reaction can be present in one batch between a few copies and several billions of copies. In case of diagnostic use the amount of the nucleic acid chain to be amplified can be unknown.

In the reaction also further nucleic acids not to be amplified can be present. These nucleic acids can be derived from natural DNA or RNA or their equivalents. In one embodiment, control sequences are present in the same batch that have to be amplified in parallel to the nucleic acid to be amplified.

Preferably, a molar excess of approximately 10³:1 to approximately 10¹⁵:1 (ratio of primer:template) of the primers used and of the activator oligonucleotide is added to the reaction mixture that comprises template strands for the synthesis of the nucleic acid chain to be amplified.

The amount of the target nucleic acids may not be known if the method according to the invention is used in diagnostic applications, so that the relative amount of the primer and of the activator oligonucleotide with respect to the complementary strand cannot certainly be determined. The amount of the primer added will generally be present in the molar excess with respect to the amount of the complementary strand (template) if the sequence to be amplified is contained in a mixture of complex long-chain nucleic acid strands. A large molar excess is preferred in order to improve efficacy of the method.

The concentrations of primer 1, primer 2 and activator oligonucleotide used are for example in ranges between 0.01 μmol/land 100 μmol/l, preferably between 0.1 μmol/land 100 μmol/l, preferably between 0.1 μmol/l and 50 μmol/l, better between 0.1 μmol/l and 20 μmol/l. The high concentration of components can increase the rate of the amplification. The respective concentrations of individual components can independently be varied in order to achieve the desired reaction result.

The concentration of polymerase is in the range between 0.001 μmol/l and 50 μmol/l, preferably between 0.01 μmol/l and 20 μmol/l, better between 0.1 μmol/l and 10 μmol/l.

The concentration of individual dNTP substrates is in ranges between 10 μmol/l and 10 mmol/l, preferably between 50 μmol/l and 2 mmol/l, better between 100 μmol/l and 1 mmol/l. The concentration of dNTP can affect the concentration of divalent metal cations. Optionally, this is correspondingly adjusted.

As divalent metal cations there are for example used Mg2+. As the corresponding anion Cl, acetate, sulphate, glutamate, etc. can be used, for example.

The concentration of divalent metal cations is adapted for example to the region that is optimal for the corresponding polymerase and comprises regions between 0.1 mmol/l and 50 mmol/l, better between 0.5 mmol/l and 20 mmol/l, preferably between 1 mmol/l and 15 mmol/l.

In general, enzymatic synthesis takes place in a buffered aqueous solution. As buffer solutions dissolved conventional buffer substances such as Tris HCl, Tris acetate, potassium glutamate, HEPES buffer, sodium glutamate in common concentrations can be used. The pH value of said solutions is usually between 7 and 9.5, preferably about 8 to 8.5. The buffer conditions may be adapted for example in accordance with the recommendations of the manufacturer of the polymerase used.

Further substances such as so-called Tm depressors (e.g., DMSO, betaines, TPAC), etc. can be added to the buffer. Such substances decrease the melting temperature (“Tm depressors”) of double strands and thus, can have a positive influence on the opening of double strands. Also, polymerase-stabilizing components such as Tween 20 or Triton 100 can be added to the buffer in the usual amounts. EDTA or EGTA can be added in conventional amounts for complexation of heavy metals. Also, polymerase-stabilizing substances such as trehalose or PEG 6000 can be added to the reaction mixture.

Preferably, the reaction mixture does not contain any inhibitors of the strand displacement reaction and no inhibitors of a polymerase-depending primer extension.

In one embodiment, the reaction mixture contains DNA-binding dyes, preferably intercalating dyes such as e.g., EvaGreen or SybrGreen. Such dyes can optionally enable the detection of the reproduction of nucleic acid chains.

The reaction mixture can further contain proteins or other substances that for example originate from an original material and that preferably do not affect the amplification.

Preferable Embodiments of Reaction Conditions

The temperature has a substantial influence on the stability of the double strands.

In a preferred embodiment, during the amplification reaction no temperature conditions are used that substantially result in a separation of double strands of the nucleic acid to be amplified in the absence of an activator oligonucleotide. In this way, it is to be ensured that the double strand separation of nucleic acid chains to be amplified depends on the presence of the activator oligonucleotide throughout the amplification.

At a temperature approximately equal to the measured melting temperature (Tm) of the nucleic acid to be amplified a spontaneous separation of both strands of the nucleic acid to be amplified occurs, so that the influence of the activator oligonucleotide on the separation of synthesized strands and thus, on the sequence specificity of the amplification is minimally limited.

In an exponential amplification that has to proceed less sequence-specifically (i.e. little activator oligonucleotide-dependent) the reaction temperature can be for example around the melting temperature (i.e. Tm plus/minus 3° C. to 5° C.) of the nucleic acid to be amplified. At such a temperature sequence differences between activator oligonucleotide and the synthesized primer extension product generally can be well tolerated during a strand displacement reaction.

Also in the temperature range of ca. (Tm minus 3° C.) to ca. (Tm minus 10° C.) there can still be a spontaneous strand separation of synthesized primer extension products, although with less efficacy. The influence of the activator oligonucleotide on the sequence specificity of the nucleic acid to be amplified is higher than at temperature conditions around the melting temperature (Tm) of the nucleic acid chain to be amplified.

Certainly, with a decreasing reaction temperature strand separation substantially takes place owing to the interaction of the re-synthesized double strand with the activator oligonucleotide, but duplexes of primers can spontaneously decompose at an extension temperature under the mentioned conditions, i.e. without sequence-dependent strand displacement by the activator oligonucleotide. For example, the reaction temperature in a less sequence-specific amplification is in ranges between ca. (Tm minus 3° C.) and ca. (Tm minus 10° C.), preferably between ca. (Tm minus 5° C.) and ca. (Tm minus 10° C.). At such a temperature sequence differences between the activator oligonucleotide and the synthesized primer extension product are tolerated less well during a strand displacement reaction.

A high sequence specificity of the amplification of the method is achieved above all when the re-synthesized strands of the nucleic acid to be amplified under reaction conditions cannot spontaneously dissociate into single strands. In such a case, sequence-specific strand displacement by the activator oligonucleotide plays a decisive role for a sequence-specific strand separation and is mainly responsible for the sequence specificity of the amplification reaction. This can generally be achieved when the reaction temperature is significantly below the melting temperature of both strands of the nucleic acid to be amplified and no further components are used for a strand separation, for example no helicases or recombinases. For example, the reaction temperature in a sequence-specific amplification is in ranges between ca. (Tm minus 10° C.) and ca. (Tm minus 50° C.), preferably between ca. (Tm minus 15° C.) and ca. (Tm minus 40° C.), better between ca. (Tm minus 15° C.) and ca. (Tm minus 30° C.).

In a preferred embodiment of the amplification the maximum reaction temperature during the whole amplification reaction will not be increased above the melting temperature of the nucleic acid chain to be amplified.

In a further embodiment of the amplification the reaction temperature can be increased above the melting temperature of the nucleic acid chains to be amplified at least once. The increase in temperature may be for example at the beginning of the amplification reaction and result in a denaturation of double strands of a genomic DNA. Here, it has to be noted that during such a step the dependency of double strand separation on the effect of the activator oligonucleotide is canceled or at least significantly reduced.

The reaction temperatures of the individual steps of the amplification reaction can be in the range of ca. 15° C. to ca. 85° C., better in the range of ca. 15° C. to ca. 75° C., preferably in the range of ca. 25° C. to ca. 70° C.

Generally, the reaction temperature can optimally be adjusted for each individual reaction step, so that for each reaction step such a temperature is brought about. Thus, the amplification reaction comprises a repeating change in temperatures that is repeated cyclically. In an advantageous embodiment of the method reaction conditions for several reaction steps are unified, so that the number of temperature steps is lower than the number of reaction steps. In such a preferred embodiment of the invention at least one of the steps of the amplification takes place at a reaction temperature that differs from the reaction temperature of other steps of the amplification. Thus, the reaction does not proceed isothermal, but the reaction temperature is cyclically changed.

For example, during amplification at least two temperature ranges are used that are mutually brought about (cyclic change in temperatures between individual temperature ranges). In one embodiment, for example the lower temperature range comprises temperatures between 25° C. and 60° C., better between 35° C. and 60° C., preferably between 50° C. and 60° C., and the upper temperature range comprises temperatures between 60° C. and 75° C., better between 60° C. and 70° C., for example.

In a further embodiment, for example the lower temperature range comprises temperatures between 15° C. and 50° C., better between 25° C. and 50° C., preferably between 30° C. and 50° C., and the upper temperature range comprises temperatures between 50° C. and 75° C., better between 50° C. and 65° C., for example.

In a further embodiment, for example the lower temperature range comprises temperatures between 15° C. and 40° C., better between 25° C. and 40° C., preferably between 30° C. and 40° C., and the upper temperature range comprises temperatures between 40° C. and 75° C., better between 40° C. and 65° C., for example.

The temperature can be maintained constant in the respective range or changed as a temperature gradient (falling or rising).

Further explanations on the temperature adjustments are given in detail in the following sections in the embodiments.

Each temperature brought about can be maintained for a certain period of time, so that in this way an incubation step results. Thus, the reaction mixture can be incubated during an amplification at a selected temperature for a certain period of time. This time can be different for the respective incubation step and can depend on the progress of the respective reaction at a given temperature (e.g., primer extension or strand displacement etc.). The time of an incubation step can comprise the following ranges: between 0.1 sec and 10.000 sec, better between 0.1 sec and 1000 sec, preferably between 1 sec and 300 sec, more preferably between 1 sec and 100 sec.

By such a temperature change individual reaction steps can preferably be carried out at a selected temperature. In this way, yields of a respective reaction step can be improved. Temperature change or temperature alteration between individual temperature ranges can optionally be brought about several times within one synthesis cycle. Thus, a synthesis cycle can comprise at least one temperature alteration. Such a temperature alteration can for example be carried out in a PCR instrument/thermocycler as a matter of routine as a time program.

In one embodiment, an amplification method is preferred in which at least one of the steps comprising strand displacement and at least one of the steps comprising primer extension reactions take place at the same time or in parallel and under the same reaction conditions. In such an embodiment, for example a primer extension reaction of at least one primer oligonucleotide (e.g., of the first primer oligonucleotide) can preferably take place at temperature conditions in the lower temperature range. In contrast, strand displacement takes place with cooperation of an activator oligonucleotide and the one further primer extension reaction (e.g., of the second primer oligonucleotide) preferably in the reaction step in the upper temperature range.

In a further embodiment, an amplification method is preferred in which at least one of the steps comprising strand displacement by the activator oligonucleotide and at least one of the steps comprising primer extension reactions are carried out at different temperatures. In such an embodiment, for example primer extension reactions of at least one primer oligonucleotide (e.g., of the first primer oligonucleotide and/or of the second primer oligonucleotide) can preferably take place at temperature conditions in the lower temperature range. In contrast, strand displacement takes place with cooperation of an activator oligonucleotide preferably in the reaction step in the upper temperature range.

In a further preferred embodiment, all the steps of an amplification reaction proceed under the same reaction conditions.

In such an embodiment, the amplification method can be carried out under isothermal conditions, i.e. no temperature changes are required to carry out the method. In such a preferred embodiment of the invention the whole amplification reaction takes place under a constant temperature, i.e. the reaction is isothermal. The duration of such a reaction comprises for example the following ranges: between 100 sec and 30.000 sec, better between 100 sec and 10.000 sec, still better between 100 sec and 1000 sec.

In section “Examples” it is shown that it is possible to adapt structures of individual reaction components and the corresponding reaction steps to each other to such an extent that an isothermal reaction is possible.

The sum of all method steps resulting in a doubling of the amount of a nucleic acid chain to be amplified can be referred to as synthesis cycle. Such a cycle can correspondingly proceed isothermal or be characterized in its course by changes of the temperature. The temperature changes can be repeated from cycle to cycle and made identical.

Of particular advantage are amplification methods in which the maximum achievable temperature only substantially allows a strand separation with the cooperation of an activator oligonucleotide if more than 5 nucleotides of the third region of the activator oligonucleotide are able to complementary bind to the first primer extension product, it is more preferred if more than 10, still more preferred if more than 20 nucleotides of the activator oligonucleotide bind to the first primer extension product. Generally, the longer the required binding between activator oligonucleotide and the complementary strand of the first primer extension product, before the synthesized strands dissociate under reaction conditions, the more specific the amplification reaction. In detail, by extending or shortening the third section of the activator oligonucleotide the desired degree of specificity can be determined.

A method step when repeated can take place at a constant temperature over the total duration of the method or also at different temperatures.

Individual method steps each can be carried out consecutively by adding individual components. In an advantageous embodiment all the reaction components required to carry out an amplification are present at the beginning of an amplification in one reaction mixture.

The start of an amplification reaction can be by adding one component, e.g., by adding a nucleic acid chain comprising a target sequence (e.g., a start nucleic acid chain), or a polymerase or divalent metal ions, or also by bringing about reaction conditions needed for amplification, e.g., adjusting a required reaction temperature for one or more method steps.

Amplification can be carried out until the desired amount of nucleic acid to be amplified has been achieved. In another embodiment, the amplification reaction is carried out for a period of time that would have been sufficient, in the presence of a nucleic acid to be amplified, to get a sufficient amount. In another embodiment, the amplification reaction is carried out over a sufficient number of synthesis cycles (duplication times) that would have been sufficient, in the presence of a nucleic acid to be amplified, to get a sufficient amount.

The reaction can be stopped by various interventions. For example, by changing the temperature (e.g., cooling or heating, wherein for example polymerase is interfered in its function) or by adding a substance that stops a polymerase reaction, e.g., EDTA or formamide.

Following the amplification the amplified nucleic acid chain can be used for further analyses. Here, synthesized nucleic acid chains can be analyzed by various detection methods. For example, fluorescence-labeled oligonucleotide probes can be used or sequencing methods (Sanger sequencing or next generation sequencing), solid phase analyses such as microarray or bead array analyses etc. The synthesized nucleic acid chain can be used as a substrate/template in further primer extension reactions.

In an advantageous embodiment, the progress of the synthesis reaction during the reaction is monitored. This can be done for example by employing intercalating dyes, e.g., SYBRgreen or Evagreen, or by employing labeled primers (e.g., Lux primers or Scorpion primers) or by employing fluorescence-labeled oligonucleotide probes.

The detection of the change in the fluorescence during the amplification is implemented in the detection step of the method. Here, the temperature and duration of said step can be adapted to the respective requirements of the oligonucleotide probe. The temperatures of the detection step for example comprise ranges between 20° C. and 75° C., better between 40 and 70° C., preferably between 55 and 70° C.

During the detection step the reaction is illuminated with light of a wavelength that is able to excite a used fluorophore of the detection system (a donor or a fluorescence reporter). Generally, signal detection is in parallel to excitation, wherein the specific fluorescence signal is detected and its intensity is quantified.

The amplification method can be applied to verify the presence of a target nucleic acid chain in a biological material or a diagnostic material during a diagnostic method.

In one embodiment reaction conditions of at least one reaction step in which at least one allele-specific primer is to hybridize to one allele-specific sequence variant and a primer extension reaction is to take place by a polymerase are chosen such that a mainly specific hybridization of such a primer to its primer binding site can take place. Such conditions may also be referred to as stringent conditions. For example, the temperature in such a step is about the Tm of the respective primer/primer binding site or the temperature is above such a Tm. For example, the temperature may be about Tm +5 to about Tm +15° C.

Preferred Embodiments of a Start Nucleic Acid Chain:

The nucleic acid chain employed or to be employed at the beginning of the amplification reaction can be referred to as a start nucleic acid chain (FIGS. 50-51).

Its function can be seen in that it represents the initial template that permits a correct positioning of primers, the synthesis sections between both primers as well as the initiation of binding and extension processes. In a preferred embodiment, a start nucleic acid chain comprises a target sequence.

By binding primers to their respective primer binding sites (PBS 1 and PBS 2) and initiating appropriate primer extension reactions first primer extension products are generated. These are synthesized as specific copies of the nucleic acid chain present at the beginning of the reaction.

In one embodiment, this nucleic acid chain (start nucleic acid chain) to be used in the reaction mixture before the beginning of the amplification reaction can be identical to the nucleic acid chain to be amplified. By the amplification reaction only the amount of such nucleic acid chain is increased.

In a further embodiment, the nucleic acid to be amplified and the start nucleic acid chain differ in that certainly the start nucleic acid chain prescribes the arrangement of individual sequence elements of the nucleic acid chain to be amplified, but the sequence composition of the start nucleic acid chain can differ from the sequence of the nucleic acid chain to be amplified. For example, in context of the primer binding and extension during an amplification new sequence contents (regarding the start nucleic acid chain) can be integrated into the nucleic acid chain to be amplified. Moreover, sequence elements of a nucleic acid chain to be amplified can differ from such sequence elements of a start nucleic acid chain in their sequence composition (e.g., primer binding sites or primer sequences). The start nucleic acid only serves as an initial template for the specific synthesis of the nucleic acid chain to be amplified.

Said initial template can remain in the reaction mixture until the end of the amplification. However, by the exponential nature of the amplification the amount of the nucleic acid chain to be amplified at the end of an amplification reaction predominates the amount of a start nucleic acid chain to be added to the reaction.

The start nucleic acid chain in one embodiment comprises a target sequence or its sub-segments that comprise at least one sequence variant of a polymorphous locus of the target sequence to be expected.

A start nucleic acid further comprises at least one mainly single-stranded sequence segment to which at least one of the primers of the amplification system can mainly complementary bind with its 3′ segment, so that polymerase used can template-specific extend such primer, when hybridized to the start nucleic acid chain, by incorporating dNTPs.

A start nucleic acid which can comprise several sequence variants in a polymorphous locus of a target sequence preferably further comprises at least one first target sequence segment which is characteristic and uniform for all the target sequence variants. In a further embodiment a start nucleic acid comprises at least two target sequence segments (a first target sequence segment and a second target sequence segment) that are located on both sides of a polymorphous locus of a target sequence and thus, flank the sequence variants of a target sequence from both sides.

The length of a polymorphous locus of a start nucleic acid chain can comprise regions from one nucleotide up to 200 nucleotides, better from one nucleotide up to 50 nucleotides, preferably from one nucleotide up to 20 nucleotides. The lengths of uniform sequence segments (first and second uniform sequence segments of a start nucleic acid) at least for one of the two uniform sequence segments can comprise the following regions: from 4 to 200 nucleotides, better from 6 to 100 nucleotides, preferably from 8 to 50 nucleotides.

In a further embodiment, the start nucleic acid chain can comprise at least one sequence part that is not amplified. Thus, such a start nucleic acid chain is identical to the sequence to be amplified. Such sections not to be amplified can represent a sequence part of a start nucleic acid chain for example as a result of sequence preparing steps or as a result of previous sequence manipulation steps, respectively.

In a preferred embodiment, the start nucleic acid chain to be added to the reaction mixture before the beginning of the reaction includes at least one target sequence.

In a further embodiment, such a start nucleic acid chain includes at least one target sequence and still further sequences that are non-target sequences. During the amplification sequence segments comprising the target sequence are exponentially multiplied and thereby, other sequence segments either are not exponentially multiplied at all or only partially.

Structure of a Start Nucleic Acid Chain

An example of such a start nucleic acid chain is a nucleic acid chain that includes a target sequence and that comprises a sequence fragment A and that comprises a sequence fragment B.

Sequence fragment A of the start nucleic acid chain comprises a sequence that has a significant homology with the sequence of one of both primers used in the amplification or is substantially identical to the copyable portion of the 3′ segment of the one primer. In the synthesis of a complementary strand to this segment a complementary sequence is generated that represents a respective primer binding site.

Sequence fragment B of the start nucleic acid chain comprises a sequence suitable to complementary bind a corresponding further primer or its 3′ segment to form an extendable primer template complex, wherein sequence fragment A and sequence fragment B with respect to each other mainly/preferably are non-complementary.

In a preferred embodiment, a start nucleic acid chain is added to the reaction mixture of an amplification method that has the following properties:

Preferably, sequence fragment A is in the 5′ segment of the start nucleic acid chain. Preferably, said sequence fragment A forms a restriction of the nucleic acid chain strand in the 5′ direction.

Preferably, sequence fragment B is downstream of sequence fragment A.

In a preferred embodiment, said sequence fragment B forms a restriction of the nucleic acid chain strand in the 3′ direction. In a further embodiment, said sequence fragment B does not represent a restriction of the nucleic acid chain strand in the 3′ direction, but is flanked by further sequences from the 3′ side. Preferably, said sequences are no target sequences and do not participate in the exponential amplification.

In one embodiment, the target sequence comprises at least one of both sequence fragment A or sequence fragment B. In a further embodiment, the target sequence is between sequence fragment A and sequence fragment B.

In one embodiment, such a start nucleic acid chain can function as a template for the synthesis of a first primer extension product (FIG. 50B). Here, the start nucleic acid chain for example during a primer extension reaction by using the second primer oligonucleotide can be provided as a sequence segment of a longer starting nucleic acid chain during a preparatory step before the exponential amplification and converted to a single-stranded form. For example, the starting nucleic acid chain can be a genomic DNA or RNA and serves as a source of the target sequence (schematically illustrated in FIG. 50A as double strand). The start nucleic acid chain in the 5′ segment of the nucleic acid chain (FIG. 50B) comprises a segment that comprises sequence parts of the second primer oligonucleotide and represents a restriction in the 5′ direction, a segment 2 that comprises a target sequence or portions thereof, a segment 3 that comprises a primer binding site for the first primer oligonucleotide, and a segment 4 that comprises a non-target sequence that is localized in the 3′ segment of the start nucleic acid chain and thus, flanks segment 3 on the 3′ side.

During the initiation of the amplification the first primer at least with the 3′ segment of its first region can bind to such a start nucleic acid chain in segment 3 (FIG. 50C) and appropriately be extended in the presence of a polymerase and nucleotides. Thereby, a first primer extension product is generated that is complementary to the template strand of the start nucleic acid chain and is restricted in its length (FIG. 50D). During the amplification reaction such a primer extension product can be sequence-specifically detached from its template strand via an activator oligonucleotide (FIG. 50 E-F), so that the corresponding sequence parts in the 3′ segment of the now synthesized first primer extension product are available as a binding site for the second primer oligonucleotide (FIG. 50 F).

In a preferred embodiment, thus a start nucleic acid chain comprises the following sequence fragments (FIG. 50):
sequence segment 1 (referred to as segment 1 in FIG. 50) that comprises a sequence that has a significant homology with the sequence of the second primer or is substantially identical to the copyable portion of the 3′ segment of the second primer. Said sequence segment 1 is in the 5′ segment of the start nucleic acid chain, preferably said sequence segment 1 forms a restriction of the nucleic acid chain strand in the 5′ direction.
sequence segment 3 (referred to as segment 3 in FIG. 50) that comprises a sequence that is suitable to complementary bind the first region of the first primer or its 3′ segment to form an extendable primer template complex. Preferably, sequence segment 3 is downstream of sequence segment 1.
target sequence that is partially or entirely between segment 1 and segment 3 (this portion of the target sequence is referred to as segment 2 in FIG. 50). In a preferred embodiment, the target sequence comprises at least one of segment 1 and/or segment 3.
optionally, the start nucleic acid chain in the 3′ segment comprises flanking sequence parts (referred to as segment 4 in FIG. 50) that are not amplified.

In a further embodiment, such a start nucleic acid chain can function as a template for the synthesis of a second primer extension product (FIG. 51).

Here, the start nucleic acid chain for example during a primer extension reaction by using the first primer oligonucleotide can be provided as a sequence segment of a longer starting nucleic acid chain (FIG. 51 A) during a preparatory step before the exponential amplification and converted to a single-stranded form. For example, the starting nucleic acid chain can be a genomic DNA or RNA and serves as a source of the target sequence (schematically illustrated in FIG. 51 A, as double strand). The start nucleic acid chain in the 5′ segment of the nucleic acid chain (FIG. 51 B) comprises a segment 5 that comprises sequence parts of the first primer oligonucleotide and represents a restriction in the 5′ direction, a segment 6 that comprises a target sequence or portions thereof, a segment 7 that comprises a primer binding site for the second primer oligonucleotide, and a segment 8 that comprises a non-target sequence that is localized in the 3′ segment of the start nucleic acid chain and thus, flanks segment 7 on the 3′ side.

During the initiation of the amplification the first primer at least with the 3′ segment of its first region can bind to such a start nucleic acid chain in segment 7 (FIG. 51 C) and appropriately be extended up to the stop section of the first primer oligonucleotide in the presence of a polymerase and nucleotides. Thereby, a second primer extension product is generated that is complementary to the template strand of the start nucleic acid chain and is restricted in its length (FIG. 51 D). During the amplification reaction such a primer extension product can be sequence-specifically detached from its template strand via an activator oligonucleotide (FIG. 51 E-F), so that the corresponding sequence parts in the 3′ segment of the now synthesized second primer extension product are available as a binding site for the first primer oligonucleotide (FIG. 51 F).

In this preferred embodiment, a start nucleic acid chain comprises the following sequence fragments (FIG. 51):
sequence segment 5 (referred to as segment 5) that comprises a sequence that has a significant homology with the sequence of the region of the first primer or is substantially identical to the copyable portion of the first region of the first primer. Said sequence segment 7 is in the 5′ segment of the start nucleic acid chain and is flanked by a non-copyable oligonucleotide tail (in analogy to the first primer oligonucleotide), preferably said sequence segment 7 forms a restriction of the copyable nucleic acid chain strand in the 5′ direction.
sequence segment 7 (referred to as segment 7) that comprises a sequence that is suitable to complementary bind a second primer or its 3′ segment to form an extendable primer template complex. Preferably, sequence segment 7 is downstream of sequence segment 5.
target sequence that is partially or entirely between segment 5 and segment 7 (in FIG. 51 this portion of the target sequence is referred to as segment 6). In a preferred embodiment, the target sequence comprises at least one of segment 5 and/or segment 7.
optionally, the start nucleic acid chain in the 3′ segment comprises flanking sequence parts (referred to as segment 8 in FIG. 51) that are not amplified.

Mode of Functioning of the Start Nucleic Acid Chain

At the beginning of the amplification reaction the start nucleic acid chain functions as a template for the initial generation of respective primer extension products. Thus, it represents the starting template for the nucleic acid chain to be amplified. The start nucleic acid chain does not necessarily have to be identical to the nucleic acid chain to be amplified. By binding and extending both primers during the amplification reaction substantially both primers prescribe which sequences on both terminal segments of the nucleic acid chain to be amplified are generated during the amplification.

In a preferred embodiment of the method reaction conditions that do not denaturize a double strand are maintained during the exponential amplification process. Hence, it is advantageous for the start nucleic acid chain to have a restriction in its 5′ sequence segment that can be extended by a polymerase, which results in a stop in the enzymatic extension of a respective primer. Thus, the length of the primer extension fragments generated under reaction conditions is restricted. This can have a beneficial effect on the strand displacement by the activator oligonucleotide and lead to a dissociation of the respective strand, so that primer binding sites are converted to the single-stranded stage and thus, become accessible for a new binding of primers.

In a further preferred embodiment of the method reaction conditions that do not denaturize a double strand are used during initial synthesis steps (for example, initial one to ten synthesis repetitions) (e.g., temperature rising up to 90° C.), however, in subsequent repetitions of the synthesis steps non-denaturizing conditions are maintained during the exponential amplification process. For such a combination of temperature conditions it is not relevant whether or not the start nucleic acid chain has a restriction in its 5′ sequence segment. By initial denaturation separation of synthesized strands is independent of their length in analogy to PCR.

Preferred Embodiments of the First Primer Oligonucleotide (Primer 1)

The first primer oligonucleotide (primer 1) is a nucleic acid chain that includes at least the following regions (FIG. 29):
a first primer region in the 3′ segment of the first primer oligonucleotide that can substantially sequence-specifically bind to a strand of a nucleic acid chain to be amplified
a second region directly or via a linker linked to the 5′ end of the first primer region of the first primer oligonucleotide that comprises a polynucleotide tail suitable to bind an activator oligonucleotide and support the strand displacement (step c) by the activator oligonucleotide, wherein the polynucleotide tail substantially remains single-stranded under the reaction conditions, i.e. does not form a stable hairpin structure or ds structures, and preferably is not copied by polymerase.

The total length of the first primer oligonucleotide is between 10 and 80, preferably between 15 and 50, better between 20 and 30 nucleotides or equivalents thereof (e.g., nucleotide modifications). The structure of the first primer oligonucleotide is adapted such that it is able to reversibly bind to the activator oligonucleotide under the selected reaction conditions. Moreover, the structure of the first primer oligonucleotide is adapted to its primer function. Moreover, the structure is adapted such that a strand displacement by means of the activator oligonucleotide can be performed. Altogether, structures of the first and second regions are adapted such that an exponential amplification can be performed.

In an advantageous embodiment of the invention the first and second regions of the primer are coupled in a conventional 5′-3′ arrangement. In a further embodiment of the invention coupling of both sections is done via a 5′-5′ bond, so that the second region has an opposite direction to the first region.

Coupling regions between each other/among each other is done preferably covalently. In one embodiment, coupling between the first and second regions is a 5′-3′ phosphodiester coupling that is conventional for DNA. In a further embodiment it is a 5′-5′ phosphodiester coupling. In a further embodiment, it is a 5′-3′ phosphodiester coupling, wherein between adjacent terminal nucleotides or nucleotide modifications of both regions at least one linker (e.g., a C3, C6, C12, or a HEG linker or an abasic modification) is positioned.

Individual regions can include different nucleotide modifications. Here, individual elements of nucleotides can be modified: nucleobase and backbone (sugar content and/or phosphate content). Moreover, there can be used modifications that lack at least one component of the standard nucleotide building blocks or are modified, e.g., PNA.

In a further embodiment, a second region of the first primer oligonucleotide comprises further sequences that do not bind to the activator oligonucleotide. These sequences can be used for other purposes, e.g., for binding to the solid phase. These sequences are preferably localized at the 5′ end of the polynucleotide tail.

In a further embodiment, a first primer oligonucleotide can comprise a characteristic label. Examples of such a label are dyes (e.g., FAM, TAMRA, Cy3, Alexa 488 etc.) or biotin or other groups that can specifically be bound, e.g., digoxigenin.

The First Primer Region of the First Primer Oligonucleotide

The sequence length is between ca. 3-30 nucleotides, preferably between 5 and 20 nucleotides, wherein the sequence is mainly complementary to the 3′ segment of a strand of the nucleic acid chain to be amplified. In detail, said primer region has to be able to specifically bind to the complementary 3′ segment of a second primer extension product. Said first region is to be copyable in backward synthesis and also functions as a template for a 2nd strand. Preferably, the nucleotide building blocks are linked among each other via common 5′-3′ phosphodiester binding or phosphothioester binding.

The first primer region preferably includes nucleotide monomers that do not or only marginally affect the function of the polymerase, these are for example:
natural nucleotides (dA, dT, dC, dG, etc.) or modifications thereof without altered base pairing
modified nucleotides, 2-amino-dA, 2-thio-dT or other nucleotide modifications with diverging base pairing.

In a preferred embodiment, the 3′-OH end of said region is preferably free from modifications and has a functional 3′-OH group that can be recognized by polymerase. The first primer region functions as an initiator of the synthesis of the first primer extension product in the amplification. In a further preferred embodiment, the first region comprises at least one phosphorothioate compound, so that no degradation of the 3′ end of the primers by 3′ exonuclease activity of polymerases can take place.

The sequence of the first region of the first primer oligonucleotide and the sequence of the second region of the activator oligonucleotide are preferably complementary to each other.

In one embodiment, the first primer region or its 3′ segment can bind to sequence segments of a target sequence.

In One Embodiment an Allele-Specific First Primer is Used in Combination with an Allele-Specific Activator Oligonucleotide.

The first primer region of the first primer can bind to the corresponding complementary position of a start nucleic acid or a nucleic acid chain to be amplified. Preferably, a first primer region comprises at least one sequence segment which can preferably specifically bind to an allele-specific sequence variant of the target nucleic acid under the reaction conditions used, wherein polymerase is able to extend a thus formed perfect-match complex, so that this results in a first primer extension product.

In one embodiment the position of an allele-specific sequence in the primer comprises the 3′-terminal nucleotide. In a further embodiment the position of an allele-specific sequence in the primer comprises the at least one of the positions −1 to −6 nucleotides in the 3′-terminal segment of the first region of the first primer. In a further embodiment the position of an allele-specific sequence in the primer comprises at least one of the positions of −6 to at least −15 in the 3′-terminal segment of the first region of the first primer.

Such a primer further comprises sequence segments which can complementary and uniformly bind to all of the allele variants of a target sequence. Thus, the first region of a first allele-specific primer comprises sequence segments that both are target sequence-specific and such that are allele-specific.

Preferably, a combination of an allele-specific primer and an allele-specific activator oligonucleotide is used, wherein the first region of the first primer oligonucleotide is completely complementary to the second region of the activator oligonucleotide.

Individual allele-specific primers may be combined in one group which covers all the variants of a target sequence. Such a group of allele-specific primers comprises at least two different allele-specific primers, since a polymorphous locus in a given position in the target sequence comprises at least two sequence variants. The allele-specific primers are constructed such that under stringent reaction conditions they preferably can form a perfect-match bond with their respective specific template and thus, use this specific perfect-match template to form the respective primer extension products under the catalytic action of the polymerase. Preferably, 3′-terminal nucleotides and/or 3′-terminal segments of allele-specific primers may be used to discriminate variants of target sequences and this way may be adapted in their sequence composition to the respective variants such that such primers with the respective variant form a perfect-match double strand under stringent conditions. Generally, such perfect-match double strands may be well recognized by a polymerase and under suitable reaction conditions primer extension takes place. Thus, if an allele-specific primer interacts with another variant of a target sequence a mismatch double strand is formed. Generally, such mismatches result in a delay of the extension by a polymerase or in a deceleration of the entire reaction. In one embodiment allele-specific primers in the 3′ segment can comprise at least one phosphorothioate bond which protects allele-specific primers against 3′-5′ nuclease decomposition by a polymerase.

Thus, several allele-specific primers comprise sequence segments which for one group of allele-specific primers are substantially identic or uniform, respectively as well as sequence segments which in the primers of one group are different and characteristic for the respective sequence variant of a target sequence. By including uniform sequence segments such primers are able to hybridize to the respective target sequence under reaction conditions. By including characteristic sequence segments, a respective primer can specifically bind to a sequence variant of the target sequence to form a perfect-match double strand. Preferably, the primers are constructed such that under the reaction conditions used binding to a target sequence to form a perfect-match double strand is preferred and binding to a target sequence to form a mismatch double strand is less preferred.

In a Further Embodiment a Target Sequence-Specific First Primer (but No Allele-Specific First Primer) is Used in Combination with an Allele-Specific Activator Oligonucleotide.

The first primer region of the first primer can bind to the corresponding complementary position of a start nucleic acid or a nucleic acid chain to be amplified. Preferably, a first primer region comprises at least one sequence segment that under the reaction conditions used preferably can sequence-specifically bind to sequence segments of a target nucleic acid chain (comprising for example a start nucleic acid and/or the nucleic acid chain to be amplified), wherein said binding substantially takes place independently of potentially present sequence differences in the polymorphous locus, wherein polymerase is able to extend a thus formed perfect-match complex, so that this results in a first primer extension product.

Binding of such a primer substantially takes place in the sequence segment of the target nucleic acid which for at least two allele variants of said target nucleic acid chains is uniform. Preferably, primer binding takes place in the sequence segment of the target nucleic acid that is uniform for all of the allele variants of said target nucleic acid.

Here, binding takes place such that the polymorphous locus of the target sequence lies in the 3′ direction from the first region of the first primer, so that in a primer extension reaction said polymorphous locus is copied by polymerase. Thus, a resulting first primer extension product comprises a complementary sequence to the polymorphous locus. This sequence lies in the 3′ direction from the first primer.

Thus, such a primer comprises sequence segments that preferably can complementary and uniformly bind to all of the allele variants of a target sequence. In order that differentiation of allele variants can take place such a primer has to be combined with at least one allele-specific activator oligonucleotide. Thus, allele discrimination takes place by the action of the activator oligonucleotide. Positioning of the polymorphous locus in the 3′ direction from the primer causes its localization in the third region of the activator oligonucleotide.

The Second Region of the First Primer Oligonucleotide

The second region of the first primer oligonucleotide is preferably a nucleic acid sequence that comprises at least one polynucleotide tail that remains preferably uncopied by polymerase during the synthesis reaction and that can bind to the first region of the activator oligonucleotide. The segment of the second region that mainly binds to the activator oligonucleotide can be referred to as polynucleotide tail.

Further, the second region of the first primer oligonucleotide not only has to specifically bind the activator oligonucleotide under reaction conditions, but also has to participate in the process of strand displacement by means of the activator oligonucleotide. Accordingly, the structure of the second region must be suitable for causing a spatial proximity between the activator oligonucleotide and the corresponding double strand end (in detail, the 3′ end of the second primer extension product).

Configuration of the structure of the second region of the first primer oligonucleotide is illustrated in detail in several embodiments. Here, the arrangement of the oligonucleotide segments and modifications used are taken into account that lead to a stop in the polymerase-catalyzed synthesis.

The length of the second region is between 3 and 60, preferably between 5 and 40, preferably between 6 and 15 nucleotides or equivalents thereof.

The sequence of the second region may be chosen arbitrarily. Preferably, it is non-complementary to the nucleic acid to be amplified and/or to the second primer oligonucleotide and/or to the first region of the first primer oligonucleotide. Moreover, it preferably does not contain any self-complementary segments such as hairpins or stem loops.

The sequence of the second region is preferably adapted to a sequence of the first region of the activator oligonucleotide, so that both sequences can bind under reaction conditions. In a preferred embodiment, said binding is reversible under reaction conditions: thus, there is an equilibrium between components bound to each other and unbound components.

The sequence of the second region of the first primer oligonucleotide is preferably selected such that the number of complementary bases that can bind to the first region of the activator oligonucleotide is between 1 and 40, better between 3 and 20, preferably between 6 and 15.

The function of the second region among others is to bind the activator oligonucleotide. In one embodiment, said binding preferably is specific, so that a second region of a first primer oligonucleotide can bind a specific activator oligonucleotide. In another embodiment, a second region can bind more than only one activator oligonucleotide under reaction conditions.

In general, there is no need for a perfect match in the sequence between the second region of the first primer oligonucleotide and the first region of the activator oligonucleotide. The degree of the complementarity between the second region of the first primer oligonucleotide and the first region of the activator oligonucleotide can be between 20% and 100%, better between 50% and 100%, preferably between 80% and 100%. The respectively complementary regions can be positioned directly adjacent to each other or also comprise non-complementary sequence segments therebetween.

In one embodiment preferably specific sequence segments are used in the second region of the first primer. These sequence-specific and thus, characteristic sequence segments preferably are not complementary to the target nucleic acid chain and are adapted to the sequence segments of the first region of an allele-specific activator oligonucleotide such that this way mainly specific complementary duplexes may be formed between the first region of the activator oligonucleotide and the second region of the first primer oligonucleotide. In this way, for example in the presence of several activator oligonucleotides (e.g., those which are allele-specific), pairing from a particular first primer and a particular activator oligonucleotide can take place. Thus, the second region of the first primer oligonucleotide can be used for a characteristic coding, particularly in multiplex analyses.

Thus, by using mainly sequence-specific sequence segments in the second primer region and at the same time using corresponding complementary sequence segments in the first region of the activator oligonucleotide potentially resulting primer extension products and characteristic activator oligonucleotides may be better assigned.

In one embodiment, the second region of the first primer oligonucleotide can include at least one Tm-modifying modification. By incorporating such modifications the stability of the bond between the second region of the first primer oligonucleotide and the first region of the activator oligonucleotide can be modified. For example, Tm-rising modifications (nucleotide modifications or non-nucleotide modifications) can be used such as LNA nucleotides, 2-amino adenosines or MGB modifications. On the other hand, also Tm-decreasing modifications can be used such as for example inosine nucleotide. In the structure of the second region also linkers (e.g., C3, C6, HEG linkers) can be integrated.

For strand displacement the activator oligonucleotide has to be brought in spatial proximity of the double strand end of the nucleic acid to be amplified. Said double strand end consists of segments of the first primer region of the first primer extension product and a correspondingly complementary 3′ segment of the second primer extension product.

The polynucleotide tail mainly complementary binds the activator oligonucleotide under reaction conditions and thus, causes a transient approximation of the second region of the activator oligonucleotide and of the first region of an extended primer extension product, so that the complementary bond between said elements can be initiated during a strand displacement process.

In one embodiment, binding of the activator oligonucleotide to the polynucleotide tail of the first primer oligonucleotide directly leads to such a contact. This means that the polynucleotide tail and the first primer region of the first primer oligonucleotide have to be directly coupled to each other. Owing to such an arrangement there may be a direct contact between complementary bases of the second region of the activator oligonucleotide and corresponding bases of the first primer region after an activator oligonucleotide has bound in its first region, so that a strand displacement can be initiated.

In a further embodiment, there are other structures of the second region of the first primer oligonucleotide between structures of the polynucleotide tail and the first primer region. Thus, after an activator oligonucleotide has bound to the polynucleotide tail this is not directly positioned to the first primer region, but in a certain distance thereto. The structures between the non-copyable polynucleotide tail and the copyable first primer region of the primer oligonucleotide can generate such a distance. Said distance has a value that is between 0.1 and 20 nm, preferably between 0.1 and 5 nm, better between 0.1 and 1 nm.

Such structures for example represent linkers (e.g., C3 or C6 or HEG linkers) or segments that are not complementary to the activator oligonucleotide (e.g., in the form of non-complementary, non-copyable nucleotide modifications). The length of these structures can generally be measured in chain atoms. Said length is between 1 and 200 atoms, preferably between 1 and 50 chain atoms, preferably between 1 and 10 chain atoms.

In order to keep the polynucleotide tail of polymerase uncopyable under amplification conditions the second region of the first primer oligonucleotide generally comprises sequence-alignments or structures, respectively that lead to a stop of the polymerase in the synthesis of the second primer extension product after the polymerase has successfully copied the first primer region. Said structures are to prevent the polynucleotide tail of the second region from being copied. Thus, the polynucleotide tail preferably remains uncopied by the polymerase.

In one embodiment, such structures are between the first primer region and the polynucleotide tail.

In a further embodiment, the sequence of the polynucleotide tail can include nucleotide modifications that lead to a stop of the polymerase. In this way, a sequence segment of the second region of the first primer oligonucleotide can comprise both functions: it is both a polynucleotide tail and a sequence of nucleotide modifications leading to a stop of the polymerase.

Modifications in the second region of the first primer oligonucleotide that lead to a synthesis stop and thus, leave the polynucleotide tail uncopied in this application are combined under the term “first blocking unit or a first stop region”.

In the Following, Further Embodiments of Structures are Given that can Lead to the Stop in the Synthesis of the Second Strand.

Several building blocks in the oligonucleotide synthesis are known that hinder polymerase from reading the template and lead to termination of the polymerase synthesis. For example, non-copyable nucleotide modifications or non-nucleotide modifications are known. There are also synthesis types/alignments of nucleotide monomers within an oligonucleotide that lead to the stop of the polymerase (e.g., 5′-5 alignment or 3′-3′ alignment). Primer oligonucleotides having a non-copyable polynucleotide tail are also known in the prior art (e.g., Scorpion primer structures or primers for binding to the solid phase). Both primer variants describe primer oligonucleotide structures that are able to initiate the synthesis of a strand, so that a primer extension reaction can take place. The result is a first strand that also integrates the primer structure with tail in the primer extension product. In the synthesis of a complementary strand to the first primer extension product, e.g., during a PCR reaction, the second strand is extended to the “blocking unit/stop structure” of the primer structure. Both described primer structures are designed such that the 5′ portion of the primer oligonucleotide remains single-stranded and is not copied by the polymerase.

In a further embodiment, the second region of the primer oligonucleotide comprises a polynucleotide tail that has a conventional alignment from 5′ to 3′ in its entire length and includes non-copyable nucleotide modifications. Such non-copyable nucleotide modifications are for example 2′-O-alkyl RNA modifications, PNA, morpholino. Said modifications can be differently distributed in the second primer region.

Non-copyable nucleotide modifications in the polynucleotide tail can share between 20% and 100%, preferably more than 50% of the nucleotide building blocks. Preferably, these nucleotide modifications are in the 3′ segment of the second region and thus, border on the first region of the first primer oligonucleotide.

In one embodiment, the sequence of the non-copyable nucleotide modifications is at least partially complementary to the sequence in the template strand, so that the primer binding to the template is done by including at least part of said nucleotide modifications. In a further embodiment, the sequence of the non-copyable nucleotide modifications is non-complementary to the sequence in the template strand.

The non-copyable nucleotide modifications are preferably covalently coupled to each other and thus, represent a sequence segment in the second region. The length of this segment comprises between 1 and 40, preferably between 1 and 20 nucleotide modifications, more preferably between 3 and 10 nucleotide modifications.

In a further embodiment, the second region of the first primer oligonucleotide comprises a polynucleotide tail that has a conventional alignment from 5′-3′ in its entire length and includes non-copyable nucleotide modifications (e.g., 2′-O-alkyl modifications) and at least one non-nucleotide linker (e.g., C3, C6, HEG linker). The function of a non-nucleotide linker is to covalently connect adjacent nucleotides or nucleotide modifications and at the same time to site-specifically interrupt the synthesis function of the polymerase.

Such a non-nucleotide linker is not to space the structures of the polynucleotide tail and of the first primer region too far from each other. Rather, the polynucleotide tail is to be in a spatial proximity to the first primer region. A non-nucleotide linker are modifications that are not longer than 200 chain atoms in their length, even more advantageous not longer than 50 chain atoms, particularly preferred not longer than 10 chain atom. The minimum length of such a linker can be one atom. An example of such non-nucleotide linkers are straight or branched alkyl linkers having an alkyl chain that includes at least one carbon atom, advantageously at least 2 to 30, more preferably 4 to 18. Such linkers are sufficiently known in the oligonucleotide chemistry (e.g., C3, C6 or C12 linkers) and can be incorporated during solid phase synthesis of oligonucleotides between the sequence of the polynucleotide tail and the sequence of the first region of the first primer oligonucleotide. Another example of such non-nucleotide linkers are linear or branched polyethylene glycol derivatives. A known example in the oligonucleotide chemistry is hexaethylene glycol (HEG). A further example of such non-nucleotide linkers are abasic modifications (e.g., THF modification, as an analogue of dRibose).

If one or more of such modifications are integrated in a second region they can effectively interfere with the copy function of a polymerase during its synthesis of the second primer extension product, so that downstream segments remain uncopied after such a modification. The number of such modifications in the second region can be between 1 and 100, preferably between 1 and 10, preferably between 1 and 3.

The position of such non-nucleotide linker can be at the 3′ end of the second region and thus, represent the transition to the first region and the second region of the primer oligonucleotide.

Also, the position of the non-nucleotide linker in the central segment of the second region can be used. Thus, a polynucleotide tail is divided into at least two segments. In this embodiment, the 3′ segment of the polynucleotide tail includes at least one, better more, e.g., between 2 and 20, preferably between 2 and 10 non-copyable nucleotide modifications. These non-copyable nucleotide modifications preferably are on the transition between the first and second regions of the primer oligonucleotide.

In a further embodiment, the second region of the primer oligonucleotide comprises a polynucleotide tail that has an alignment from 5′ to 3′ in its total length and includes at least one nucleotide monomer in an “reverse” alignment from 3′ to 5′ and that are positioned at the transition between the first and second regions of the first primer oligonucleotide.

In a further embodiment, the second region of the primer oligonucleotide comprises a polynucleotide tail, wherein such a polynucleotide tail completely consists of nucleotides that directly border on the first region of the first primer oligonucleotide in a reversed alignment, so that the coupling of the first and second regions is by the 5′-5′ position. An advantage of such an alignment is that the polymerase after having copied the first region encounters a “reverse” alignment of nucleotides, which typically leads to the termination of the synthesis at this site.

In a “reverse” alignment of nucleotides in the total length of the polynucleotide tail preferably the 3′-terminal nucleotide of the polynucleotide tail is to be blocked at its 3′-OH end in order to prevent side reactions. Alternatively, also a terminal nucleotide can be used that has no 3′-OH groups at all, e.g., a dideoxynucleotide.

In such an embodiment, of course also the corresponding nucleotide alignment in the activator oligonucleotide is to be adapted. In such a case, the first and second regions of the activator oligonucleotide have to be linked in a 3′-3′ alignment.

In a further embodiment, the second region of the primer oligonucleotide comprises a polynucleotide tail that has a conventional alignment from 5′ to 3′ in its total length and includes at least one nucleotide modification that does not represent a complementary nucleobase to the polymerase if the synthesis is performed exclusively with natural dNTPs (dATP, dCTP, dGTP, dTTP, or dUTP).

For example, Iso-dG or Iso-dC nucleotide modifications can be integrated in the second region of the first primer oligonucleotide as single, but preferably several (at least 2 to 20) nucleotide modifications. Further examples of nucleobase modifications are various modifications of the extended genetic alphabet. Such nucleotide modifications preferably do not support a complementary base pairing with natural nucleotides, so that a polymerase (at least theoretically) does not insert a nucleotide from the series (dATP, dCTP, dGTP, dTTP, or dUTP). In reality, however there may be a rudimentary insertion, especially at higher concentrations of dNTP substrates and prolonged incubation times (e.g., 60 min or longer). Hence, preferably several of such nucleotide modifications positioned at adjacent sites are to be employed. The stop of the polymerase synthesis is effected by lacking appropriate complementary substrates for these modifications. Oligonucleotides having Iso-dC or Iso-dG can be synthesized with standard methods and are available from several commercial suppliers (e.g., Trilink-Technologies, Eurogentec, Biomers GmbH). Alternatively, also the sequence of the first region of the activator oligonucleotide can be adapted to the sequence of such a second primer region. Here, complementary nucleobases of the extended genetic alphabet can accordingly be integrated in the first region of the activator oligonucleotide during the chemical synthesis. For example, Iso-dG can be integrated in the second region of the first primer nucleotide, its complementary nucleotide (Iso-dC-S-Me) can be placed at the appropriate site in the first region of the activator oligonucleotide.

In summary, the termination of the synthesis of polymerase in the second region may be achieved in different manners. However, this blockage preferably only takes place when the polymerase has copied the first region of the first primer oligonucleotide. In this way it is ensured that a second primer extension product has an appropriate primer binding site in its 3′ segment. This primer binding site is exposed during the strand displacement and thus, is available for a new binding of a further first primer oligonucleotide.

In the synthesis of the complementary strand to the first primer extension product the primer extension reaction stops before the polynucleotide tail. Since in this way this polynucleotide tail remains single-stranded for interaction with the activator oligonucleotide and thus, is available for binding it supports the initiation of the strand displacement reaction by the activator oligonucleotide by bringing the corresponding complementary segments of the activator oligonucleotide in close proximity to the appropriate duplex end. In this way, the distance between the complementary part of the activator oligonucleotide (second region) and the complementary part of the extended primer oligonucleotide (first region) is reduced to a minimum. Such a spatial proximity facilitates the initiation of the strand displacement.

In the context of a schematic illustration of a nucleic acid-mediated strand displacement reaction now a complementary sequence of an activator oligonucleotide is in close proximity of the appropriate duplex end. This results in competition for the binding to the first region of the first primer oligonucleotide between the strand of the activator oligonucleotide and the template strand complementary to the primer. By repetitively closing and forming base pairing between the primer region and the complementary segment of the activator oligonucleotide (second region of the activator nucleotide) or the complementary segment of the template strand, respectively initiation of the nucleic acid-mediated strand displacement process occurs.

Generally, the yield of the initiation of the strand displacement is the higher the closer the corresponding complementary sequence part of the activator oligonucleotide is to the complementary segment of the primer region. However, when this distance is increased the yield of the initiation of the strand displacement decreases.

In the context of the present invention it is not mandatory that the initiation of the strand displacement works at the maximum yield. Thus, distances between the 5′ segment of the first primer region of the first primer oligonucleotide, that binds to a complementary strand of the template and forms a complementary duplex, and a corresponding complementary sequence part in the activator oligonucleotide when bound to the polynucleotide tail of the second region of the first primer oligonucleotide may be in the following ranges: between 0.1 and 20 nm, better between 0.1 and 5 nm, even better between 0.1 and 1 nm. In the preferred case, said distance is less than 1 nm. Expressed in other units said distance corresponds to a track of less than 200 atoms, even better less than 50 atoms, even better less than 10 atoms. In the preferred case, said distance is one atom. The distance information is for orientation only and to illustrate that shorter distances between these structures are preferred. In many cases, said distance can only be measured by analyzing the exact structures of oligonucleotides and evaluating the measurement of sequence distances or linker lengths.

The first primer may also comprise further sequence parts that are not needed for an interaction with the activator oligonucleotide or the template strand. Such sequence parts for example can bind further oligonucleotides that are used as detection probes or immobilization partners in the binding to the solid phase.

Primer Function of the First Primer Oligonucleotide

The first primer oligonucleotide may be used in several individual steps. First of all, it exerts a primer function in the amplification. Thereby, a primer extension reaction is performed using the second primer extension product as a template. In a further embodiment, the first primer oligonucleotide at the beginning of the amplification reaction can use the start nucleic acid chain as template. In a further embodiment, the first primer oligonucleotide can be used in designing/providing a start nucleic acid chain.

During the amplification the first primer functions as an initiator of the synthesis of the first primer extension product using the second primer extension product as a template. The 3′ segment of the first primer comprises a sequence that can mainly complementary bind to the second primer extension product. The enzymatic extension of the first primer oligonucleotide using the second primer extension product as a template results in the formation of the first primer extension product. Such a first primer extension product comprises the target sequence or sequence portions thereof. In the course of the synthesis of the second primer extension product the sequence of the copyable portion of the first primer oligonucleotide is recognized by polymerase as a template and a corresponding complementary sequence is synthesized, so that a respective primer binding site results for the first primer oligonucleotide. Synthesis of the first primer extension product is up to and including the 5′ segment of the second primer oligonucleotide. Immediately following synthesis of the first primer extension product said product is bound to the second primer extension product and forms a double-stranded complex. The second primer extension product is sequence-specifically displaced from said complex by the activator oligonucleotide. Thereby, the activator oligonucleotide binds to the first primer extension product. Following a successful strand displacement by the activator oligonucleotide the second primer extension product in turn itself can function as a template for the synthesis of the first primer extension product. The now free 3′ segment of the first primer extension product can bind a further second primer oligonucleotide, so that a new synthesis of the second primer extension product can be initiated.

Moreover, the first primer oligonucleotide can function as an initiator of the synthesis of the first primer extension product starting from the start nucleic acid chain at the beginning of the amplification. In one embodiment, the sequence of the first primer is completely complementary to the corresponding sequence segment of a start nucleic acid chain. In a further embodiment, the sequence of the first primer oligonucleotide is only partially complementary to the corresponding sequence segment of a start nucleic acid chain. However, said diverging complementarity is not to prevent the first primer oligonucleotide from starting a mainly sequence-specific primer extension reaction. The respective differences in complementarity of the first primer oligonucleotide to the respective position in the start nucleic acid chain are preferably in the 5′ segment of the first region of the first primer oligonucleotide, so that in the 3′ segment mainly complementary base pairing and initiation of the synthesis is possible. For the initiation of the synthesis for example especially the first 4-10 positions in the 3′ segment are to be completely complementary to the template (start nucleic acid chain). The remaining nucleotide positions may diverge from a perfect complementarity. Thus, the degree of a perfect complementarity in the remaining 5′ segment of the first region of the first primer oligonucleotide can comprise ranges between 50% to 100%, better between 80% and 100% of the base composition. According to the length of the first region of the first primer oligonucleotide the sequence divergences are 1 to at most 15 positions, better 1 to at most 5 positions. Thus, the first primer oligonucleotide can initiate a synthesis of a start nucleic acid chain. In a subsequent synthesis of the second primer extension product copyable sequence parts of the first primer oligonucleotide are copied by polymerase, so that in turn in subsequent synthesis cycles a completely complementary primer binding site is formed within the second primer extension product for the binding of the first primer oligonucleotide and is available in subsequent synthesis cycles.

In a further embodiment, the first primer oligonucleotide can be used in the preparation of a start nucleic acid chain. Thereby, such a first primer oligonucleotide can mainly/preferably sequence-specifically bind to a nucleic acid (e.g., a single-stranded genomic DNA or RNA or equivalents thereof comprising a target sequence) and initiate a template-dependent primer extension reaction in the presence of a polymerase. The binding position is selected such that the primer extension product comprises a desired target sequence. Extension of the first primer oligonucleotide results in a nucleic acid strand that has a sequence complementary to a template. Such a strand can be detached from the template (e.g., by heat or alkali) and thus, converted to a single-stranded form. Such a single-stranded nucleic acid chain can function as a start nucleic acid chain at the beginning of the amplification. Such a start nucleic acid chain in its 5′ segment comprises the sequence portions of the first primer oligonucleotide, further it comprises a target sequence or equivalents thereof and a primer binding site for the second primer oligonucleotide. Further steps are explained in section “start nucleic acid chain”.

The synthesis of the first primer extension product is a primer extension reaction and forms an individual step in the amplification. The reaction conditions during this step are accordingly adapted. Reaction temperature and reaction time are selected such that the reaction can successively take place. The preferred temperature in this step depends on the polymerase used and the binding strength of the respective first primer oligonucleotide to its primer binding site and comprises for example ranges of 15° C. to 75° C., better of 20 to 65° C., preferably of 25° C. to 65° C. The concentration of the first primer oligonucleotide comprises ranges of 0.01 μmol/l to 50 μmol/l, better of 0.1 μmol/l to 20 μmol/l, preferably of 0.1 μmol/l to 10 μmol/l.

In one embodiment, all steps of the amplification proceed under stringent conditions that prevent or delay the formation of non-specific products/by-products. Such conditions are for example higher temperatures, for example above 50° C.

If more than one specific nucleic acid chain is to be amplified in one batch, in one embodiment, preferably sequence-specific primer oligonucleotides are used for amplification of the corresponding target sequences.

Preferably, sequences of the first, the second primer oligonucleotides and of the activator oligonucleotide are adapted to each other such that side reactions, e.g., primer dimer formation, are minimized. For that, for example the sequences of the first and second primer oligonucleotides are adapted to each other such that both primer oligonucleotides are not able to start or support, respectively an amplification reaction in the absence of an appropriate template and/or a target sequence and/or a start nucleic acid chain. This can be achieved for example in that the second primer oligonucleotide does not comprise a primer binding site for the first primer oligonucleotide and the first primer oligonucleotide does not comprise a primer binding site for the second primer oligonucleotide. Moreover, it is to be avoided that the primer sequences comprise extended self-complementary structures (self-complement).

In one embodiment, the synthesis of the first and second primer extension products proceeds at the same temperature. In a further embodiment, the synthesis of the first and second primer extension products proceeds at different temperatures. In a further embodiment, synthesis of the first primer extension product and strand displacement by the activator oligonucleotide proceed at the same temperature. In a further embodiment, synthesis of the first primer extension product and strand displacement by the activator oligonucleotide proceed at different temperatures.

Use of a first primer oligonucleotide in combination with a first competitor primer oligonucleotide:

Preferred Embodiment of the First Competitor Primer Oligonucleotide (Primer-1)

A first competitor primer oligonucleotide with the first primer oligonucleotide competes for the binding to a sequence segment of a nucleic acid chain comprising a target sequence that comprises at least two variants of a target nucleic acid chain.

Here, the first primer oligonucleotide binds to the desired or expected sequence variant and is extended by a polymerase preferably under stringent reaction conditions. The first competitor oligonucleotide binds to the second potential or expected sequence variant and is extended by a polymerase preferably under stringent reaction conditions. Thus, two primer extension products are formed: the one of the first primer oligonucleotide which is to be further propagated in the amplification and the other one of the first competitor primer oligonucleotide which is not to be propagated in the amplification.

By the competition of two primers for one primer binding sequence in the target nucleic acid chain specificity of the analysis can be improved.

Thus, the two primers (the first primer and the first competitor primer) represent allele-specific primers with respect to potential or expected sequence variants. The substantial difference is mainly in that the competitor primer is not able to initiate a strand separation with the help of an allele-specific activator oligonucleotide.

In one embodiment a first competitor primer oligonucleotide (Competitor Primer-1) comprises a nucleic acid chain including at least the following properties:
a first competitor primer region in the 3′ segment of the first competitor primer oligonucleotide that can substantially specifically bind to a strand of a nucleic acid chain to be amplified and thus, can competitively act with the first primer oligonucleotide for its primer binding site.
3′ end of the competitor primer which can sequence-specifically be extended by polymerase after binding to the primer binding site in the target sequence to form a competitor primer extension product.
3′ end of the competitor which is prevented from extension by a polymerase by the “second blocking unit” and/or by the “fourth blocking unit” of the activator oligonucleotide, when the first competitor primer oligonucleotide is complementary bound to the activator oligonucleotide.

The total length of the first competitor primer oligonucleotide is between 10 and 80, preferably between 15 and 50, better between 20 and 30 nucleotides or their equivalents (e.g., nucleotide modifications). The length of the 3′ segment which can form a complementary bond to a sequence variant of a target sequence to be expected comprises substantially the same regions like the first region of the first primer. In this way it is ensured that both primers can competitively bind to the target sequence and said binding takes place in about the same temperature range. The competitive bond and above all competitive primer extension using allele-specific sequence segments of target nucleic acid generally results in a further increase of the specificity.

The structure of the first competitor primer oligonucleotide is adapted such that under the chosen reaction conditions it can reversible bind to an activator oligonucleotide. In one embodiment the first competitor primer comprises sequence segments which shall form no complementary bond with the first region of the activator oligonucleotide. Thus, said first competitor oligonucleotide cannot represent an initiator of a strand displacement by an activator oligonucleotide.

In an advantageous embodiment of the invention the first competitor primer oligonucleotide is a DNA oligonucleotide having a conventional 5′-3′ arrangement of nucleotides.

In a further embodiment a competitor primer oligonucleotide comprises further sequences which do not bind to the activator oligonucleotide. Said sequences may be used for other purposes, e.g., for binding to the solid phase. Said sequences are preferably located at the 5′ end of the 3′ segment.

The First Primer Region of the First Competitor Primer Oligonucleotide

The sequence length is between ca. 15-30 nucleotides, preferably between 5 and 20 nucleotides, wherein the sequence is mainly complementary to the 3′ segment of a strand of the allele variant of a target nucleic acid to be suppressed in the amplification. The first primer region preferably includes nucleotide monomers that do not or only marginally affect the function of the polymerase, these are for example:
natural nucleotides (dA, dT, dC, dG etc.) or modifications thereof without altered base pairing
modified nucleotides, 2-amino-dA, 2-thio-dT or other nucleotide modifications with diverging base pairing.

In one embodiment an allele-specific first primer is used in combination with an allele-specific activator oligonucleotide and an allele-specific competitor primer oligonucleotide.

Preferred Embodiments of the Activator Oligonucleotide

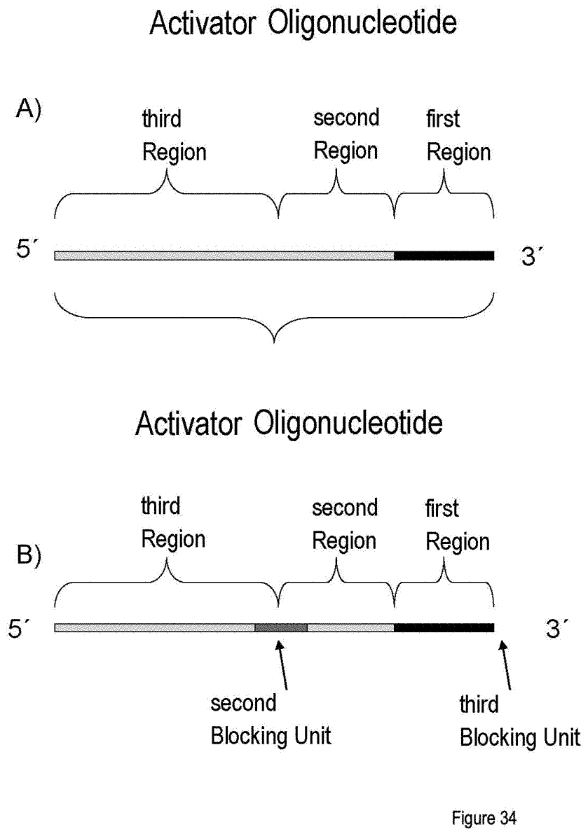

An activator oligonucleotide (FIG. 34) comprises:
a first single-stranded region that can bind to the polynucleotide tail of the second region of the first primer oligonucleotide,
a second single-stranded region that can substantially complementary bind to the first region of the first primer oligonucleotide,
a third single-stranded region that is substantially complementary at least to one segment of the extension product of the first primer extension product,
and the activator oligonucleotide does not function as a template for primer extension of the first or second primer oligonucleotides.

In general, the sequence of the third region of the activator oligonucleotide is adapted to the sequence of the nucleic acid to be amplified, since this is relevant as a template for the order of the nucleotides in the extension product of a first primer. The sequence of the second region of the activator oligonucleotide is adapted to the sequence of the first primer region. The structure of the first region of the activator oligonucleotide is adapted to the sequence of the second region of the first primer oligonucleotide, especially to the nature of the polynucleotide tail.

An activator oligonucleotide can also include further sequence segments that do not belong to the first, second or third regions. These sequences can be attached for example as flanking sequences to the 3′ and 5′ end. Preferably, these sequence segments do not interfere with the function of the activator oligonucleotide.

The structure of the activator oligonucleotide preferably has the following properties:

The individual regions are covalently bound among each other. Binding for example can be via conventional 5′-3′ binding. For example, a phosphodiester binding or nuclease-resistant phosphothioester binding may be used.

An activator oligonucleotide can bind to the polynucleotide tail of the first primer oligonucleotide by means of its first region, wherein binding is mainly mediated by hybridizing complementary bases. The length of said first region is 3-80 nucleotides, preferably 4-40 nucleotides, particularly preferred 6-20 nucleotides. The degree of sequence matching between the sequence of the first region of the activator oligonucleotide and the sequence of the second region of the first primer oligonucleotide can be between 20% and 100%, preferably between 50% and 100%, particularly preferred between 80% and 100%. Binding of the first region of the activator oligonucleotide preferably is to be specific to the second region of the first primer oligonucleotide under reaction conditions.

The sequence of the first region of the activator oligonucleotide is preferably selected such that the number of complementary bases that can complementary bind to the second region of the first primer oligonucleotide is between 1 and 40, better between 3 and 20, preferably between 6 and 15.

Since the activator oligonucleotide does not represent a template for polymerase it can include nucleotide modifications that do not support the polymerase function that can be both base modifications and/or sugar phosphate backbone modifications. The activator oligonucleotide in its first region can for example include nucleotide and/or nucleotide modifications that are selected from the following list: DNA, RNA, LNA (“locked nucleic acids” analogues with 2′-4′ bridge-type binding in the sugar residue), UNA (“unlocked nucleic acids” without a binding between 2′-3′ atoms of the sugar residue), PNA (“peptide nucleic acids” analogues), PTO (phosphorothioate), morpholino analogues, 2′-O-alkyl RNA modifications (such as 2′-OMe, 2′-0 propargyl, 2′-O-(2-methoxyethyl), 2′-O-propyl-amine), 2′-halo RNA, 2′-amino RNA etc. These nucleotides or nucleotide modifications are linked to each other for example by a conventional 5′-3′ binding or 5′-2′ binding. For example, a phosphodiester binding or nuclease-resistant phosphothioester binding can be used.

The activator oligonucleotide in its first region can include nucleotides and/or nucleotide modifications, wherein the nucleobases are selected from the following list: adenine and analogues thereof, guanine and analogues thereof, cytosine and analogues thereof, uracil and analogues thereof, thymine and analogues thereof, inosine or other universal bases (e.g., nitroindol), 2-amino-adenine and analogues thereof, iso-cytosine and analogues thereof, iso-guanine and analogues thereof.

The activator oligonucleotide in its first region can include non-nucleotide compounds that are selected from the following list: intercalating substances that can affect the binding strength between the activator oligonucleotide and the first primer oligonucleotide, e.g., MGB, naphthalene etc. The same elements can also be used in the second region of the first primer.

The activator oligonucleotide in its first region can include non-nucleotide compounds, e.g., linkers such as C3, C6, HEG linkers that can link individual segments of the first region to each other.

The activator oligonucleotide can bind to the first primer region of the first primer oligonucleotide by means of its second region, wherein binding is substantially mediated by the hybridization of complementary bases.

The length of the second region of the activator oligonucleotide is adapted to the length of the first region of the first primer oligonucleotide and preferably corresponds to it. It is between ca. 3-30 nucleotides, preferably between 5 and 20 nucleotides. The sequence of the second region of the activator oligonucleotide is preferably complementary to the first region of the first primer oligonucleotide. The degree of matching in complementarity is between 80% and 100%, preferably between 95% and 100%, preferably 100%. The second region of the activator oligonucleotide preferably includes nucleotide modifications that prevent polymerase in the extension of the first primer oligonucleotide, but do not block or substantially prevent formation of complementary double strands, for example 2′-O-alkyl RNA analogues (e.g., 2′-O-Me, 2′-O-(2-methoxyethyl), 2′-O-propyl, 2′-O-propargyl nucleotide modifications), LNA, PNA or morpholino nucleotide modifications. Individual nucleotide monomers are preferably linked via a 5′-3′ binding, but alternatively also a 5′-2′ binding between nucleotide monomers can be used.

The sequence length and its nature of the first and second regions of the activator oligonucleotide are preferably selected such that binding of said regions to the first primer oligonucleotide under reaction conditions at least in one reaction step of the method is reversible. That is, that the activator oligonucleotide and the first primer oligonucleotide certainly can specifically bind to each other, but this binding is not to result in the formation of a complex of both elements that is permanently stable under reaction conditions.

Rather, an equilibrium between a bound complex form of activator oligonucleotide and first primer oligonucleotide and a free form of individual components is to be intended or enabled under reaction conditions at least in one reaction step. In this way it is ensured that at least part of the first primer oligonucleotides under reaction conditions is present in a free form and can interact with the template to initiate a primer extension reaction. On the other hand, in this way it is ensured that the respective sequence regions of the activator oligonucleotides are available for binding with an extended primer oligonucleotide.

By selecting the temperature during the reaction the portion of free, single-stranded and thus, reactive components can be affected: by decreasing the temperature first primer oligonucleotides bind to the activator oligonucleotides, so that both participants bind a complementary double-stranded complex. In this way, the concentration of single-stranded forms of individual components can be reduced. An increase of the temperature can result in the dissociation of both components in a single-stranded form. In the range of the melting temperature of the complex (activator oligonucleotide/first primer oligonucleotide) ca. 50% of the components are present in the single-stranded form and ca. 50% as a double-stranded complex. Thus, by using appropriate temperatures the concentration of single-stranded forms in the reaction mixture can be affected.

In embodiments of the amplification method that include a change in temperature between individual reaction steps the desired reaction conditions can be effected during the respective reaction steps. For example, by using temperature ranges of about the melting temperatures of complexes of activator oligonucleotide/first primer oligonucleotide portions of free forms of individual components can be affected. Here, the temperature used results in destabilization of complexes comprising activator oligonucleotide/first primer oligonucleotide, so that during this reaction step individual complex components at least transiently become single-stranded and thus, are enabled to interact with other reaction partners. For example, the first sequence region of the activator oligonucleotide can be released from the double-stranded complex with a non-extended first primer and thus, interact with the second sequence region of an extended first primer oligonucleotide and thus, initiate a strand displacement. On the other hand, the release of a first, non-extended primer oligonucleotide from a complex comprising activator oligonucleotide/first primer oligonucleotide results in that the first primer region becomes single-stranded and thus, can interact with the template, so that a primer extension by a polymerase can be initiated.

Here, the temperature used must exactly correspond to the melting temperature of the complex of activator oligonucleotide/first primer oligonucleotide. It is sufficient if the temperature in one reaction step is used about in the range of the melting temperature. For example, the temperature in one of the reaction steps comprises ranges of Tm±10° C., better Tm±5° C., preferably Tm±3° C. of the complex of activator oligonucleotide/first primer oligonucleotide.

Such a temperature can be adjusted for example during the reaction step that comprises a sequence-specific strand displacement by the activator oligonucleotide.

In embodiments of the amplification method that do not comprise a change in temperature between individual reaction steps and where amplification proceeds under isothermal conditions reaction conditions are maintained for the entire duration of the amplification reaction under which an equilibrium between a complex form of activator oligonucleotide and the first primer oligonucleotide and a free form of individual components is possible.

The ratio between a complex form of activator oligonucleotide and the first primer oligonucleotide and free forms of individual components can be affected both by reaction conditions (e.g., temperature and Mg2+ concentration) and by the structures and concentrations of the individual components.

The sequence length and its nature of the first and second region of the activator oligonucleotide in one embodiment are selected such that under given reaction conditions (e.g., in the reaction step of a strand displacement by the activator oligonucleotide) the ratio between a portion of a free activator oligonucleotide and a portion of an activator oligonucleotide in a complex with a first primer oligonucleotide comprises the following ranges: of 1:100 to 100:1, preferably of 1:30 to 30:1, particularly preferred of 1:10 to 10:1. The ratio between a portion of a free first primer oligonucleotide and a portion of a first primer oligonucleotide in a complex with an activator oligonucleotide comprises ranges of 1:100 to 100:1, preferably of 1:30 to 30:1, particularly preferred of 1:10 to 10:1.

In one embodiment, the concentration of the first primer oligonucleotide is higher than the concentration of the activator oligonucleotide. In this way, there is an excess of the first primer in the reaction and the activator oligonucleotide, for its effect, has to be released from the binding with the first primer by selecting appropriate reaction temperatures. In general, this is done by raising the temperature until sufficient concentrations of free forms of the activator oligonucleotide are present.

In a further embodiment, the concentration of the first primer oligonucleotide is lower than the concentration of the activator oligonucleotide. In this way, there is an excess of the activator oligonucleotide and the first primer oligonucleotide, for its effect, has to be detached from the binding with the activator oligonucleotide by selecting appropriate reaction temperatures. In general, this is done by raising the temperature until sufficient concentrations of free forms of the first primer oligonucleotide are present.

With isothermal conditions there is an equilibrium: certain portions of the first primer oligonucleotide and activator oligonucleotide are bound to each other, whereas others are present as a single-stranded form in the reaction.

The activator oligonucleotide can bind to at least one segment of the specifically synthesized extension product of the first primer oligonucleotide by means of its third region. Binding is preferably done by the hybridization of complementary bases between the activator oligonucleotide and the extension product synthesized by polymerase.

In order to support the strand displacement reaction the sequence of the third region preferably is to have a high complementarity to the extension product. In one embodiment, 100% of the sequence of the third region is complementary to the extension product.

Preferably, the third region binds to the segment of the extension product that immediately follows the first region of the first primer oligonucleotide. Thus, the segment of the extension product preferably is in the 5′ segment of the total extension product of the first primer oligonucleotide.

Preferably, the third region of the activator oligonucleotide is not bound over the entire length of the extension product of the first primer oligonucleotide. Preferably, one segment at the 3′ end of the extension product remains unbound. Said 3′-terminal segment is needed for the binding of the second primer oligonucleotide.

The length of the third region is accordingly adapted such that the third region binds to the 5′-standing segment of the extension product, but does not bind the 3′-standing segment of the extension product.

The total length of the third region of the activator oligonucleotide is 2 to 100, preferably 6 to 60, particularly preferred 10 to 40 nucleotides or equivalents thereof. The activator oligonucleotide can complementary bind to the segment of the extension product over this length and thus, displace this 5′-standing segment of the extension product from the binding with its complementary template strand.

The length of the 3′-standing segment of the extension product that is not bound by the activator oligonucleotide comprises for example ranges between 5 and 200, better 5 and 100, still better between 5 and 60 nucleotides, preferably between 10 and 40, preferably between 15 and 30 nucleotides.

Said 3′-standing segment of the extension product is not displaced by the activator oligonucleotide from the binding with the template strand. Also in case of a completely bound third region of the activator oligonucleotide to its complementary segment of the extension product the first primer extension product can remain bound with the template strand via its 3′-standing segment. The binding strength of said complex is preferably selected such that it can for example spontaneously dissociate under reaction conditions (step e)). This can be achieved for example in that the melting temperature of said complex of the 3′-standing segment of the extension product of the first primer oligonucleotide and its template strand is about in the range of the reaction temperature or below the reaction temperature in a respective reaction step (reaction step e). In case of a low stability of said complex in the 3′ segment of the extension product a complete binding of the third region of the activator oligonucleotide to the 5′-standing segment of the extension product results in a rapid dissociation of the first primer extension product from its template strand.

Altogether, the activator oligonucleotide has an appropriate structure to exert its function: under the respective reaction conditions it is able to sequence-specifically displace the extended first primer oligonucleotide from the binding with the template strand, whereby the template strand is converted to the single-stranded form and thus, is available for further bindings with a new first primer oligonucleotide and its target sequence-specific extension by polymerase.

In order to fulfill the function of the strand displacement regions one, two and three of the activator oligonucleotide are mainly to be present in the single-stranded form under reaction conditions. Hence, double-stranded self-complementary structures (e.g., hairpins) in these regions are to be avoided, if possible, since they can lower the functionality of the activator oligonucleotide.

In the method according to the invention the activator oligonucleotide is not to be present as a template, hence the first primer oligonucleotide, when attached to the activator oligonucleotide under reaction conditions, is not to be extended by polymerase.

This is preferably achieved by the use of nucleotide modifications that prevent polymerase from copying the strand. Preferably, the 3′ end of the first primer oligonucleotide remains unextended if the first primer oligonucleotide binds to the activator oligonucleotide under reaction conditions.

The extent of blockage/hindrance/deceleration/complication of the reaction can be between a full expression of this property (e.g., 100% blockage under given reaction conditions) and a partial expression of this property (e.g., 30-90% blockage under given reaction conditions). Preferred are nucleotide modifications that alone or coupled in series (e.g., as a sequence fragment consisting of modified nucleotides) can prevent the extension of a first primer more than 70%, preferably more than 90%, more preferably more than 95%, and particularly preferred 100%.

The nucleotide modifications can comprise base modifications and/or sugar phosphate residue modifications. Sugar phosphate modifications are preferred, since by a combination with conventional nucleobases an arbitrary complementary sequence of an activator oligonucleotide can be arranged. The nucleotide with modifications in the sugar phosphate residue that can result in the hindrance or blockage of the synthesis of the polymerase, for example includes: 2′-O-alkyl modifications (e.g., 2′-O-methyl, 2′-O-(2-methoxyethyl), 2′-O-propyl, 2′-O-propargyl nucleotide modifications), 2′-amino-2′-deoxy-nucleotide modifications, 2′-amino-alkyl-2′-deoxy-nucleotide modifications, PNA, morpholino modifications etc.

Blockage can be both by a single nucleotide modification or only by coupling several nucleotide modifications in series (e.g., as a sequence fragment consisting of modified nucleotides). For example, at least 2, preferably at least 5, particularly preferred at least 10 of such nucleotide modifications can be coupled next to each other in the activator oligonucleotide.

An activator oligonucleotide can have a uniform type of nucleotide modifications or comprise at least two different types of nucleotide modification.

The position of such nucleotide modifications in the activator oligonucleotide preferably is to prevent the polymerase from extending the 3′ end of a first primer oligonucleotide bound to the activator oligonucleotide.

In one embodiment, such nucleotide modifications are located in the second region of the activator oligonucleotide. In a further embodiment, such nucleotide modifications are located in the third region of the activator oligonucleotide. In a further embodiment, such nucleotide modifications are located in the second and in the third regions of the activator oligonucleotide.

For example, at least 20%, preferably at least 50% of the positions of the second region of the activator oligonucleotide consist of such nucleotide modifications.

For example, at least 20%, preferably at least 50%, particularly preferred at least 90% of the positions of the third region of the activator oligonucleotide consist of such nucleotide modifications. In one embodiment the entire third region comprises nucleotide modifications which prevent a polymerase from extending a primer bound to such a region using the activator oligonucleotide as a template. In a further embodiment the entire third and second regions comprise such nucleotide modifications. In a further embodiment the entire first, second, and third regions comprise such nucleotide modifications. Thus, the activator oligonucleotide can completely consist of such nucleotide modifications. Such modified activator oligonucleotides may for example be used in multiplex analyses in which further primers are used. In this way it shall be prevented that unintentional primer extension reactions take place on one or more activator oligonucleotides.

The sequence of the nucleobases of these nucleotide modifications is adapted to the demands on the sequence in the respective region.

The rest are for example natural nucleotide or nucleotide modifications that do not hinder polymerase function at all or only marginally, e.g., DNA nucleotides, PTO nucleotides, LNA nucleotides, RNA nucleotides. Here, further modifications, for example base modifications such as 2-amino-adenosine, 2-aminopurines, 5-methyl-cytosines, inosines, 5-nitroindoles, 7-deaza-adenosine, 7-deaza-guanosine, 5-propyl-cytosine, 5-propyl-uridine or non-nucleotide modifications such as dyes, or MGB modifications etc. can be used e.g., to adjust binding strength of individual regions of the activator oligonucleotide. The individual nucleotide monomers can be coupled to each other via a conventional 5′-3′ binding or also else via a 5′-2′ binding.

A segment of the activator oligonucleotide with nucleotide modifications that prevent an extension of the 3′ end of a first primer oligonucleotide bound to an activator oligonucleotide by polymerase is referred to as “second blocking unit”. The length of said segment can include between 1 to 50 nucleotide modifications, preferably between 4 and 30. Said segment can be located in the activator oligonucleotide for example such that the 3′ end of the bound first primer oligonucleotide is in this segment. Thus, this segment can span regions two and three. When using further primers (e.g., competitor primers) which can also complementary bind to the activator oligonucleotide further blocking units may be inserted, for example a fourth blocking unit which may lie in the same position in the activator oligonucleotide like the second blocking unit or comprises a different position. For example, the fourth blocking unit may lie in the 5′ direction from the second blocking unit. Primer blockage by such a fourth blocking unit takes place in accordance with the same principle like with the second blocking unit: a primer which is able to complementary bind to the activator oligonucleotide is not extended by polymerase. Thus, it is prevented that the activator oligonucleotide for further primers, e.g., a competitor primer, is used as a template.

In one embodiment, preferably no linker structures or spacer structures such as C3, C6, HEG linkers are used to prevent extension of the 3′ end of a first primer oligonucleotide bound to the activator oligonucleotide.

The activator oligonucleotide in addition to regions one, two and three can also comprise further sequence segments that flank the above-mentioned regions for example in the 5′ segment or 3′ segment of the activator oligonucleotide. Such sequence elements can be used for example for further functions such as for example interaction with probes, binding to solid phase etc. Such regions preferably do not interfere with the function of regions one to three. The length of these flanking sequences may be for example between 1 to 50 nucleotides. Moreover, an activator oligonucleotide can comprise at least one element for immobilization to a solid phase, e.g., a biotin residue. Moreover, an activator oligonucleotide can comprise at least one element for detection, e.g., a fluorescent dye.

In the presence of an activator oligonucleotide re-synthesized sequences are examined in view of their sequence contents by interaction with the activator oligonucleotide.
In case of a correct matching with given sequence contents of the activator oligonucleotide strand displacement occurs, wherein the template strands are replaced by re-synthesized strands. Thereby, primer binding sites are converted to a single-stranded state and are available for a new interaction with primers and thus, for a further synthesis. Thus, the system of both primer extension products is put into an active state. Thus, the activator oligonucleotide has an activating effect on the system.
In case of a lacking matching with given sequence contents of the activator oligonucleotide strand displacement of the template strands of re-synthesized strands is affected. Strand displacement and/or detachment either is quantitatively decelerated or completely cancelled. Thus, primer binding sites are not converted to the single-stranded state at all or less often. Thus, no primer binding sites at all or quantitatively less are available for a new interaction with primers. Thus, the system of both primer extension products is less often put into an active state or an active state is not achieved at all.

Efficacy of the double strand opening of the re-synthesized primer extension products after each single synthesis step has an effect on the potentially obtainable yields in subsequent cycles: the less free/single-stranded primer binding sites are provided to a nucleic acid chain to be amplified at the beginning of a synthesis step, the smaller is the number of re-synthesized strands of the nucleic acid chain to be amplified in this step. In other words: The yield of a synthesis cycle is proportional to the amount of primer binding sites that are available for the interaction with the corresponding complementary primers. Altogether, in this way a control loop can be realized.

Said control loop corresponds to a real-time/on-line control of synthesized fragments: sequence control is performed in the reaction mixture while the amplification takes place. Said sequence control is performed in accordance with a given pattern and the oligonucleotide system (by a strand-opening effect of the activator oligonucleotide) is able to distinguish between “correct” and “incorrect” states without external interventions. In the correct state the synthesis of sequences is continued, in the incorrect state synthesis is either decelerated or completely prevented. The resulting differences in the yields of “correct” and “incorrect” sequences after each step have an effect on the whole amplification that comprises a number of such steps.

In an exponential amplification said dependency is exponential, so that even minor divergences in efficacy in one single synthesis cycle due to sequence divergences can mean a significant delay in time of the whole amplification or cause a complete absence of a detectable amplification in a given time frame.

This effect of the real-time control of the re-synthesized nucleic acid chains is associated with the employment of the activator oligonucleotide and thus, the influence of the activator oligonucleotide during an amplification significantly goes beyond the length of primer oligonucleotides.

Sequence Variant-Specific Activator Oligonucleotides:

The target sequence-specific activator oligonucleotide in its regions is preferably constructed such that it is able to contribute to the amplification of at least one pre-defined sequence variant of a target sequence.

In a preferred form an amplification system comprising at least one activator oligonucleotide is designed such that the position of an allele variant of a target sequence to be expected corresponds to a respective segment or corresponding position in the activator oligonucleotide.

An allele-specific activator oligonucleotide is preferably constructed such that it is able to preferably cause the amplification of a sequence variant of a target sequence (e.g., allele 1), wherein with another sequence variant of the same target sequence (e.g., allele 2) amplification does not take place at all or only with reduced efficiency or does not take place at all in the given time or results in a yield of amplification products that is not sufficient for a detection.

Thus, an allele-specific activator oligonucleotide not only is provided target sequence-specific, but also allele-specific. Thus, such an activator oligonucleotide comprises both target sequence-specific sequence segments and allele-specific sequence segments. Thus, depending on the complexity of a polymorphous locus of a target sequence several activator oligonucleotides specific for respective allele variants of a target sequence may be constructed.

Preferably, an activator oligonucleotide comprises at least one sequence segment which has a sequence composition complementary to a specific allele variant of a target sequence. In addition, an activator oligonucleotide preferably comprises at least one sequence segment comprising a complementary sequence uniform for all sequence variants of a target sequence. In one embodiment an activator oligonucleotide comprises at least one sequence segment which can complementary bind to a variant of a polymorphous locus of a target sequence.

In one embodiment several activator oligonucleotides are provided that comprise allele-specific sequence segments.

The length of a sequence segment of an activator oligonucleotide complementary to the polymorphous locus of a start nucleic acid chain can comprise regions from one nucleotide up to 100 nucleotides, better from one nucleotide up to 50 nucleotides, preferably from one nucleotide up to 20 nucleotides. The length of at least one sequence segment of an activator oligonucleotide which is complementary to uniform sequence segments of a target sequence (first and second uniform sequence segments of a start nucleic acid) can comprise the following regions: from 4 to 100 nucleotides, better from 6 to 100 nucleotides, preferably from 8 to 50 nucleotides.

In one embodiment a sequence segment of an activator oligonucleotide which is complementary to at least one variant of a polymorphous locus of a target sequence is located in the third region of the activator oligonucleotide.

In a further embodiment a sequence segment of an activator oligonucleotide which is complementary to at least one variant of a polymorphous locus of a target sequence is located in the second region of the activator oligonucleotide.

In a further embodiment a sequence segment of an activator oligonucleotide which is complementary to at least one variant of a polymorphous locus of a target sequence partially and/or fully is located in the second and the third region of the activator oligonucleotide.

Thus, the position of allele-specific sequence segments of an activator oligonucleotide may be divided into three groups.

The first group comprises allele-specific sequence segments that are detected both in the first primer oligonucleotide and in the activator oligonucleotide. Thus, said position at least partially comprises the second region of the activator oligonucleotide and the first region of the first primer oligonucleotide.

The second group comprises allele-specific corresponding sequence segments that are only covered by the activator oligonucleotide, both primers (the first primer and the second primer) are only target sequence-specific, but not allele-specific. Thus, the position at least in part comprises the third region of the activator oligonucleotide which is to complementary bind with the 5′ segment of the portion of the first primer extension product synthesized by polymerase.

The third group comprises allele-specific sequence segments that are detected both in the second primer oligonucleotide and in the activator oligonucleotide. Thus, said position at least partially comprises the third region of the activator oligonucleotide mainly in the 5′ segment of the activator oligonucleotide.

Discrimination between allele variants of a target sequence in these groups comprises different mechanisms which have to be considered for example in choosing the reaction conditions

In the first and third groups primers play an essential role in discrimination. Complementary primer binding to a corresponding primer binding site of an allele variant of the target sequence and initiation of a synthesis of a primer extension product by the polymerase determine the extent of the different amplifications. The activator oligonucleotide only interacts with the first primer (first group) and with the complementary sequence segment to the 3′ segment of the second primer (third group). Thus, successful strand displacement by the activator oligonucleotide most of all depends on the corresponding complementary design of said portions of primers and of the activator oligonucleotide. The synthesized segment portion of the respective primer extension product that lies between both primers represents a uniform sequence composition for one allele variant of a common target sequence. For that reason, it is advantageous to carry out amplification at stringent hybridization conditions that promote an allele-specific initiation of a synthesis. Using a further allele-specific competitor primer may further improve a specific initiation of the synthesis.

In the second group initiation of the synthesis of primer extension products plays rather a secondary role, since the primers used bind to primer binding sites which represent a uniform composition for all the potential allele variants of a target sequence. Discrimination only takes place with the help of the activator oligonucleotide which contributes to the separation of the first and second primer extension products by sequence-specific or mainly specific strand displacement.

In one embodiment preferably a fully complementary sequence of the third region of the activator oligonucleotide is used which can form a perfect match with at least one allele variant of the target sequence. Thus, amplification of different allele variants in this embodiment requires a combination of one allele-specific activator oligonucleotide each.

In general, variation from fully complementary sequence compositions results in varying amplification efficiencies. On the corresponding sequence segment of an activator oligonucleotide competition for the binding to the first primer extension product (mainly consisting of DNA) with the second primer extension product (also comprising DNA) takes place. Allele-specific sequence design of said sequence segment of the activator oligonucleotide corresponding to an allele variant for both competing strands enables a perfect-match/perfect-match binding to the first primer extension product. Thus, an activator oligonucleotide is able to successfully displace the second primer extension product from the binding with the first primer extension product with its corresponding sequence segment. Separation of both primer extension products enables an exponential amplification.

In case of a variation in the composition of the corresponding segment of the activator oligonucleotide (e.g., in case of a binding to a non-complementary allele variant of a primer extension product) generally the situation changes. Interaction between perfect-match of the second primer extension product (consisting of DNA) and of the first primer extension product (consisting of DNA) may only hardly or not at all be overcome by a segment of an activator oligonucleotide that comprises a mismatch. Thus, by a preferred binding of a perfect-match double strand between both primer extension products there is no sufficient strand separation with the help of a mismatch activator oligonucleotide, what generally results in an insufficient or decelerated amplification.

In a preferred embodiment the sequence segment corresponding to an allele variant lies in the third region of the activator oligonucleotide which mainly or exclusively comprises DNA monomers.

In a further embodiment the sequence segment corresponding to an allele variant lies in the third region of the activator oligonucleotide that comprises both DNA and DNA modifications.

In a further embodiment the sequence segment corresponding to an allele variant lies in the third region of the activator oligonucleotide that mainly or exclusively comprises DNA modifications. For example, in locating a sequence segment corresponding to an allele variant within a second blocking unit which comprises several 2′-O-alkyl modifications of nucleotides such modifications may act on the binding of the activator oligonucleotide to allele-specific variants of primer extension products.

In a preferred embodiment the segment of an activator oligonucleotide corresponding to an allele variant lies in the third region of the activator oligonucleotide, wherein said segment of the strand of the activator oligonucleotide is mainly composed of DNA nucleotides. Preferably, said segment lies in the 5′ direction from the second blocking unit. The length of said segment corresponding to the polymorphous locus preferably comprises 1 to 20 nucleotides. Here, the composition of the nucleotides in said fully complementary corresponding segment of the activator oligonucleotide comprises at least 70% of DNA nucleotides, better 80%, preferably more than 95% of DNA nucleotides.

Preferably, also with an allele variant which comprises only one nucleobase (e.g., SNP or a point mutation) the corresponding position lies in the middle region of the activator oligonucleotide strand which on both sides to said corresponding position is surrounded by DNA monomers complementary to the target sequence, wherein the strand of the activator oligonucleotide comprises at least 4 DNA monomers, better at least 6 DNA nucleotides, preferably at least 10, in both directions from the SNP or point mutation, respectively, to be expected. Such an arrangement of DNA monomers around SNP or point mutation, respectively, to be expected or single nucleotide allele variant enables maintenance of a single strand conformation that is characteristic for the B form (so-called single strand persistence length). Thus, strand displacement by the activator oligonucleotide takes place using a corresponding segment comprising DNA monomers.

In one embodiment use is made of an activator oligonucleotide that with its 5′ end can form a complementary bond with the first primer extension product. With allele variants comprising only one nucleobase, e.g., SNP or point mutations, the corresponding sequence segment is preferably arranged in a distance from the 5′ end of the activator oligonucleotide that comprises lengths of 10 to 60 nucleotides, better 20 to 50 nucleotides, preferably 30 to 40 nucleotides.

However, the use of DNA nucleotides as monomers of activator oligonucleotides must not result in the fact that the activator oligonucleotide is used as a template for a primer extension using one of the primers employed by a polymerase used.

The use of DNA nucleotides (for example, A, C, T, or G) within the segment corresponding to an allele variant in the activator oligonucleotide strand has several advantages in that the discrimination behavior of an amplification system is easier to asses.

For example, the behavior of perfect-match variants of an activator oligonucleotide over certain allele variants in point mutations may be easier to asses. Here, discrimination between allele variants follows widely recognized rules of Watson-Crick base pairing: activator oligonucleotides which can form complementary base pairs (A:T, G:C) with the first primer extension products generally lead to amplification reactions of good yields.

On the other hand, potential mismatches between an allele-specific activator oligonucleotide and a first primer extension product (e.g., A:C or G:G) lead to an insufficient or delayed amplification. In the presence of allele variants which can make up more than one nucleotide difference to the activator oligonucleotide in a potential first primer extension product (e.g., short InDels) generally no sufficient amplification is achieved.

The effect of a mismatch on an amplification may be assessed by investigating stabilities of potential duplexes comprising an allele-specific activator oligonucleotide having a defined sequence and a potential primer extension product or its portions, e.g., only the synthesized portion of a primer extension product. The duplexes are formed in the absence of second primer extension products. In the hybridization of an allele-specific activator oligonucleotide to a potential primer extension product with complete complementarity (perfect match) binding of such duplexes is more stable than the binding of duplexes comprising at least one mismatch position. Said stability can be detected by means of melting temperature measurement. Generally, differences between amplification reactions of allele variants are the greater the greater the difference in the stability between a perfect-match duplex and a mismatch duplex comprising an allele-specific activator oligonucleotide and a potential first primer extension product is. Such differences between reactions may be measured for example by the time required for the reaction until a certain amount of products is achieved. Such differences in time between amplification reactions of perfect-match and mismatch allele variants may also be regarded as the ability for discrimination: the greater the time difference the higher the discrimination between individual allele variants.

Due to use of modifications in the activator oligonucleotide, e.g., in the region of the second blocking unit, to prevent extension of the first primer or a competitor primer, varying discrimination or even tolerance over certain mismatches may take place. Here, for example 2′-O-alkyl modifications within the second blocking unit can be used. Such nucleotide modifications can change the conformation of a double strand (from the B form of a DNA:DNA duplex to the A-like form of a DNA:RNA or DNA: mod. DNA duplex). Here, most of all a change of the discrimination can take place in the base pairing between G:C and G:U or G:T, respectively.

For example, the use of modifications in the activator oligonucleotide leads to a change in the discrimination behavior in single base pairs. Similar behavior is known from the research of RNA: so, the behavior of G:U changes depending on the strand form: with RNA the G:U base pair generally forms a sufficiently stable base pairing, with DNA a G:T mismatch generally leads to a weakening of the binding of both strands.

In this application, for example modifications comprising 2′-alkyl nucleotides are used in the activator oligonucleotide (e.g., within the second blocking unit). Here, a 2′-O-Me cytosine (C*) or 2′-O-Me adenosine (A*) is used. Such modified nucleotides enable a good discrimination between individual sequence variants (wherein perfect match binding comprises C*:dG and A*:U or A*:T and mismatch comprises e.g., C*:dA or A*:dC).

On the other hand, the use of 2′-O-Me-G (G*) or 2′-O-Me-U (U*) leads to varying results. For example, an amplification using activator oligonucleotides having a G* at the corresponding site in the strand at the same time leads to the amplification of allele variants having dC and dU at corresponding sites in the first primer extension product.

Thus, not only allele-discriminating, but also an allele-tolerant function of an activator oligonucleotide can be illustrated.

Thus, when using nucleotide modifications that allow an insufficient discrimination of allele variants, several allele variants may be amplified at the same time. In addition of a 2′-O-Me guanosine or 2′-O-Me uridine also universal bases, such as inosine or 5-nitro-indole monomers are among the candidates with tolerance over the allele composition. Such modifications of an activator oligonucleotide strand may be arranged in expected corresponding positions to certain allele variants. In the presence of one of the allele variants an amplification propagating at least two different allele variants can take place.

Thus, said lacking discrimination in some modifications can be considered as follows:

In a further embodiment the sequence segment corresponding to an allele variant lies in the third region of the activator oligonucleotide that mainly or exclusively comprises 2′-O-alkyl modifications.

In a preferred embodiment the segment of an activator oligonucleotide corresponding to an allele variant lies in the third region of the activator oligonucleotide, wherein said segment of the strand of the activator oligonucleotide is mainly composed of 2′-O-alkyl modifications. Preferably, said segment lies within the second blocking unit or in the 5′ direction of the second blocking unit. The length of said segment corresponding to the polymorphous locus preferably comprises 1 to 20 nucleotides. Here, the composition of the nucleotides in said fully complementary corresponding segment of the activator oligonucleotide comprises at least 50% of 2′-O-alkyl modifications, better 80%, preferably more than 95% of 2′-O-alkyl modifications.

Preferably, also with an allele variant which comprises only one nucleobase (e.g., SNP or a point mutation) the corresponding position lies in the middle region of the activator oligonucleotide strand which on both sides to said corresponding position is surrounded by 2′-O-alkyl modifications complementary to the target sequence, wherein the strand of the activator oligonucleotide comprises at least four 2′-O-alkyl modifications, better at least six 2′-O-alkyl modifications, preferably at least 10 2′-O-alkyl modifications in both directions from the expected SNP or point mutation, respectively. Such an arrangement of DNA monomers around an expected SNP site or point mutation or single nucleotide allele variant enables the maintenance of a single strand conformation which can be referred to as an A-like form. Thus, strand displacement by the activator oligonucleotide takes place using a corresponding segment which comprises nucleotides having 2′-O-alkyl modifications.

In one embodiment use is made of an activator oligonucleotide that with its 5′ end can form a complementary bond with the first primer extension product. With allele variants comprising only one nucleobase, e.g., SNP or point mutations, the corresponding sequence segment is preferably arranged in a distance from the 5′ end of the activator oligonucleotide that comprises lengths of 10 to 60 nucleotides, better 20 to 50 nucleotides, preferably 30 to 40 nucleotides.

In locating a sequence segment to be expected to an allele variant within such a segment comprising several 2′-O-alkyl modifications optionally varying base pairing is considered. With a difference between allele variants of only one nucleotide the following rule is applied:

Activator oligonucleotides comprising 2′-O-alkyl modifications of cytosine generally can form sufficient base pairing with dG nucleotides at corresponding sites in the first primer extension product and thus, support the amplification of such allele variants.

Activator oligonucleotides comprising 2′-O-alkyl modifications of adenosine generally can form sufficient base pairing with dT or dU nucleotides at corresponding sites in the first primer extension product and thus, support the amplification of such allele variants.

Activator oligonucleotides comprising 2′-O-alkyl modifications of guanosine generally can form sufficient base pairing with dC and dU nucleotides at corresponding sites in the first primer extension product and thus, support the amplification of both allele variants.

Activator oligonucleotides comprising 2′-O-alkyl modifications of uridine generally can form sufficient base pairing with dA and dG nucleotides at corresponding sites in the first primer extension product and thus, support the amplification of both allele variants.

Due to a strand complementarity of a double-stranded target nucleic acid, strand-specific amplification systems can be created, wherein at the corresponding sites either 2′-O-alkyl modifications of cytosine or 2′-O-alkyl modifications of adenosine, respectively, are used. Thus, lacking discrimination of G and U nucleotide analogues can be avoided.

Reaction Conditions at Strand Displacement Reaction

The displacement of the second primer extension product from the binding with the first primer extension product by means of a sequence-dependent strand displacement by the activator oligonucleotide forms an individual step in the amplification. The reaction conditions during said step are accordingly adapted. Reaction temperature and reaction time are selected such that the reaction can successfully take place.

In a preferred embodiment strand displacement by the activator oligonucleotide is up to detachment/dissociation of the second primer extension product from the binding with the first primer extension product. Such a dissociation of the 3′ segment of the first primer extension product of complementary portions of the second primer extension product can be spontaneous during a temperature-dependent/temperature-related separation of both primer extension products. Such a dissociation has a favorable effect on the kinetics of the amplification reaction and can be affected by the choice of the reaction conditions, e.g., by means of temperature conditions. Therefore, temperature conditions are selected such that a successful strand displacement by complementary binding of the activator oligonucleotide favors a dissociation of the second primer extension product from the 3′ segment of the first primer extension product.

In a further preferred embodiment strand displacement by the activator oligonucleotide proceeds up to the detachment/dissociation of a 3′ segment of the second primer extension product (P2.1-Ext) from the complementary binding with the first primer extension product (P1.1-Ext), wherein said 3′ segment of the second primer extension product (P2.1-Ext) comprises at least one complementary region to the first primer and a complementary segment to the first primer extension product (P1.1-Ext), which has only been formed in the enzymatic synthesis. Here, a complex (C1.1/P1.1-Ext/P2.1.-Ext) is formed (FIG. 1B), which comprises both the first primer extension product (P1.1-Ext), the second primer extension product (P2.1-Ext) as well as the activator oligonucleotide (C1.1). In such a complex the 3′ segment of the second primer extension product at least temporarily is present in a single-stranded form, since it can be displaced from binding with the first primer extension product by the activator oligonucleotide. Schematically illustrated there is a balance between the binding and detachment of the P2.1-Ext to and from the P1.1-Ext. The 3′ segment of the first primer extension product (P1.1-Ext) in such a complex is present complementary hybridized to the second primer extension product (P2.1-Ext) (FIG. 1B).

Due to a partially/temporary free primer binding site for the first primer oligonucleotide (3′ segment of the second primer extension product) a new primer extension product (P1.2) can bind to said single-stranded sequence segment of the (P2.1-Ext), which is still in the complex, under reaction conditions and thus, initiate a synthesis of a new first primer extension product (P1.2-Ext) by a polymerase. Generally, initiation of said reaction proceeds with reduced efficiency, since the 3′ segment of the P2.1-Ext is not permanently present in the single-stranded form, but is in a competitive behavior with the activator oligonucleotide and thus, alternatingly has single-stranded and double-stranded states by the binding to the P1.1-Ext.

Continuation of said re-started synthesis of P1.2-Ext using the second primer extension product as a template (P2.1-Ext) may also contribute to/result in the dissociation of the complexes (C1.1/P1.1-Ext/P2.1.-Ext) by strand displacement associated with polymerase. Here, activator oligonucleotide, temperature-dependent double strand destabilization and strand displacement by the polymerase act synergistically and complementary. The result is a dissociation of the 3′ segment of the first primer extension product (P1.1-Ext) from complementary portions of the second primer extension product (P2.1-Ext).

Such a dissociation has a favorable effect on the kinetics of the amplification reaction and may be influenced by the choice of the reaction conditions, e.g., by means of temperature conditions. Contribution of the polymerase-mediated synthesis-depending strand displacement to the dissociation of P1.1-Ext and P2.1-Ext has a favorable effect in strand separation.

The temperature in this step comprises for example ranges of 15° C. to 75° C., better of 30° C. to 70° C., preferably of 50° C. to 70° C.

With a given length of the first region of the activator oligonucleotide and the second region of the first primer oligonucleotide (comprising for example ranges of 3 to 25 nucleotide monomers, better of 5 to 15 nucleotide monomers) a strand displacement reaction generally can successfully be initiated. In case of a complete complementarity of the activator oligonucleotide to the respective portions of the first primer extension product the activator oligonucleotide can bind to the first primer extension product except for the 3′ segment of the first primer extension product and displace the second primer extension product. Thus, the second primer extension product remains connected to the 3′ segment of the first primer extension product. The strength of said connection can be affected depending on temperature. When reaching a critical temperature this connection can disintegrate and both primer extension products can dissociate. The shorter the sequence of the 3′ segment, the more instable the connection and the lower the temperature that causes a spontaneous dissociation.

A spontaneous dissociation can for example be achieved in the temperature range that is about the melting temperature. In one embodiment, the temperature of the steps of strand displacement by the activator oligonucleotide is about at the melting temperature (Tm±3° C.) of the complex comprising the 3′ segment of the first primer extension product that is not bound by the activator oligonucleotide and the second primer oligonucleotide or the second primer extension product, respectively.

In one embodiment, the temperature of the steps of strand displacement by the activator oligonucleotide is at about the melting temperature (Tm±5° C.) of the complex comprising the 3′ segment of the first primer extension product that is not bound by the activator oligonucleotide and the second primer oligonucleotide or the second primer extension product, respectively.

In one embodiment, the temperature of the steps of strand displacement by the activator oligonucleotide is above the melting temperature of the complex comprising the 3′ segment of the first primer extension product that is not bound by the activator oligonucleotide and the second primer oligonucleotide or the second primer extension product, respectively. Such a temperature comprises temperature ranges of about Tm+5° C. to Tm+20° C., better of Tm+5° C. to Tm+10° C. By using a higher temperature the equilibrium in said reaction step can be shifted toward dissociation. Thereby, the kinetics of the reaction can favorably be influenced. Using too low temperatures in the step of strand displacement by means of the activator oligonucleotide can lead to a significant deceleration of the amplification.

In one embodiment, a first primer extension product comprises a 3′ segment that is not bound by the activator oligonucleotide and that comprises sequence lengths of 9 to about 18 nucleotides. In this embodiment, a spontaneous dissociation in general can already be achieved with temperature ranges between 40° C. and 65° C. Also higher temperatures lead to dissociation.

In one embodiment, a first primer extension product comprises a 3′ segment that is not bound by the activator oligonucleotide and that comprises sequence lengths of 15 to about 25 nucleotides. In this embodiment, a spontaneous dissociation in general can already be achieved with temperature ranges between 50° C. and 70° C. Also higher temperatures lead to dissociation.

In one embodiment, a first primer extension product comprises a 3′ segment that is not bound by the activator oligonucleotide and that comprises sequence lengths of 20 to about 40 nucleotides. In this embodiment, a spontaneous dissociation in general can already be achieved with temperature ranges between 50° C. and 75° C. Also higher temperatures lead to dissociation.

The composition of the 3′ segment of the first primer extension product and optionally adding melting temperature-affecting oligonucleotide modifications (e.g., MGB) or reaction conditions (e.g., TPAC, betaines) can have effect on the choice of the temperature. A corresponding adjustment can therefore be made.

In one embodiment, all steps of the amplification proceed under stringent conditions that prevent or decelerate the formation of non-specific products/by-products. Such conditions are for example higher temperatures, for example above 50° C.

In one embodiment, the individual steps of strand displacement by the activator oligonucleotides proceed at the same temperature such as the synthesis of the first and second primer extension products. In a further embodiment, the individual steps of strand displacement by the activator oligonucleotides proceed at a temperature that differs from the temperature of the respective synthesis of the first and second primer extension products. In a further embodiment, the synthesis of the first primer extension product and strand displacement by the activator oligonucleotide proceed at the same temperature. In a further embodiment, synthesis of the second primer extension product and strand displacement by the activator oligonucleotide proceed at the same temperature.

The concentration of the activator oligonucleotide comprises ranges of 0.01 μmol/l to 50 μmol/l, better of 0.1 μmol/l to 20 μmol/l, preferably of 0.1 μmol/l to 10 μmol/l.

Preferred Embodiments of the Second Primer Oligonucleotide (Primer 2):

An oligonucleotide that with its 3′ segment is able to bind to a substantially complementary sequence within the nucleic acid to be amplified or equivalents thereof and to initiate a specific second primer extension reaction (FIG. 37). This second primer oligonucleotide thus is able to bind to the 3′ segment of a first specific primer extension product of the first primer oligonucleotide and to initiate a polymerase-dependent synthesis of a second primer extension product. In one embodiment, each second primer oligonucleotide is specific for one nucleic acid to be amplified each.

The second primer oligonucleotide is to be copyable upon backward synthesis and also functions as a template itself during the synthesis of the first primer extension product.

The length of the second primer oligonucleotide can be between 15 and 100 nucleotides, preferably between 20 and 60 nucleotides, particularly preferred between 30 and 50 nucleotides. The nucleotide building blocks are preferably linked to each other via common 5′-3′ phosphodiester binding or phosphothioester binding. Such a primer oligonucleotide can be chemically synthesized in the desired manner.

In one embodiment, the second primer oligonucleotide can include nucleotide monomers that do not or only insignificantly influence the function of polymerase, these are for example:
natural nucleotides (dA, dT, dC, dG etc.) or their modifications without changed base pairing
modified nucleotides, 2-amino-dA, 2-thio-dT or other nucleotide modifications with diverging base pairing (e.g., universal base pairs such as for example inosine 5-nitroindole).

In a preferred embodiment, the 3′-OH end of this region is preferably free from modifications and has a functional 3′-OH group that is recognized by polymerase and can be extended dependent on a template. In a further preferred embodiment, the 3′ segment of the second primer comprises at least one phosphorothioate compound, so that the 3′ end of the primer cannot be degraded by the 3′ exonuclease activity of polymerases.

The second primer oligonucleotide can be used in several individual steps. First, it exerts a primer function in the amplification. Thereby, a primer extension reaction using the first primer extension product as a template is performed. In a further embodiment, the second primer oligonucleotide can use the start nucleic acid chain as a template at the beginning of the amplification reaction. In a further embodiment, the second primer oligonucleotide can be used in designing/providing a start nucleic acid chain.

During the amplification the second primer functions as an initiator of the synthesis of the second primer extension product using the first primer extension product as a template. The 3′ segment of the second primer comprises a sequence that can mainly complementary bind to the first primer extension product. The enzymatic extension of the second primer oligonucleotide using the first primer extension product as a template leads to the formation of the second primer extension product. Such a synthesis typically takes place in parallel to the displacement of the activator oligonucleotide from its binding with the first primer extension product. Said displacement mainly is by polymerase and can partially be done by the second primer oligonucleotide. Such a second extension product comprises the target sequence or segments thereof. In the course of the synthesis of the second primer extension product the sequence of the copyable portion of the first primer oligonucleotide is recognized by polymerase as template and a respective complementary sequence is synthesized. Said sequence is in the 3′ segment of the second primer extension product and comprises a primer binding site for the first primer oligonucleotide. The synthesis of the second primer extension product is up to the stop position in the first primer oligonucleotide. Immediately after the synthesis of the second primer extension product this product is bound to the first primer extension product and forms a double-stranded complex. The second primer extension product is sequence-specifically displaced from said complex by the activator oligonucleotide. After a successful strand displacement by the activator oligonucleotide the second primer extension product itself in turn can function as a template for the synthesis of the first primer extension product.

Moreover, the second primer oligonucleotide can function as an initiator of the synthesis of the second primer extension product starting from the start nucleic acid chain at the beginning of the amplification. In one embodiment, the sequence of the second primer is completely complementary to the corresponding sequence segment of a start nucleic acid chain. In a further embodiment, the sequence of the second primer oligonucleotide is only partially complementary to the corresponding sequence segment of a start nucleic acid chain. However, said diverging complementarity is not to prevent the second primer oligonucleotide from starting a mainly sequence-specific primer extension reaction. The respective divergences in complementarity of the second primer oligonucleotide to the respective position in the start nucleic acid chain are preferably in the 5′ segment of the second primer oligonucleotide, so that in the 3′ segment mainly complementary base pairing and initiation of the synthesis are possible. For the initiation of the synthesis for example particularly the first 4-10 positions in the 3′ segment are to be fully complementary to the template (start nucleic acid chain). The remaining nucleotide positions can diverge from perfect complementarity. Thus, the degree of a perfect complementarity in the 5′ segment can comprise ranges of 10% to 100%, better between 30% and 100% of the base composition. Depending on the length of the second primer oligonucleotide these divergences from a full complementarity in the 5′ segment comprise from 1 to 40, better 1 to 20 nucleotide positions. In a further embodiment, the second primer oligonucleotide binds to the start nucleic acid chain only with its 3′ segment, but not with its 5′ segment. The length of such a 3′ segment of the second primer oligonucleotide that is completely complementary to the start nucleic acid chain comprises ranges between 6 and 40 nucleotides, better between 6 and 30 nucleotides, preferably between 6 and 20. The length of a corresponding 5′ segment of the second primer oligonucleotide that is non-complementary to the start nucleic acid chain comprises ranges between 5 and 60, better between 10 and 40 nucleotides. Thus, the second primer oligonucleotide is able to initiate the synthesis of a start nucleic acid chain. In a subsequent synthesis of the first primer extension product sequence parts of the second primer oligonucleotide are copied by polymerase, so that in turn in subsequent synthesis cycles a completely complementary primer binding site is formed within the first primer extension product for binding of the second primer oligonucleotide and is available in subsequent synthesis cycles.

In a further embodiment, the second primer oligonucleotide can be used during the preparation of a start nucleic acid chain. Here, such a second primer oligonucleotide can mainly/preferably sequence-specifically bind to a nucleic acid (e.g., a single-stranded genomic DNA or RNA or equivalents thereof comprising a target sequence) and initiate a template-depending primer extension reaction in the presence of a polymerase. The binding position is selected such that the primer extension product comprises a desired target sequence. Extending the second primer oligonucleotide results in a strand that has a sequence complementary to the template. Such a strand can be detached by the template (e.g., by heat or alkali) and so converted into a single-stranded form. Such a single-stranded nucleic acid chain can function as a start nucleic acid chain at the beginning of the amplification. Such a start nucleic acid chain comprises in its 5′ segment the sequence portions of the second primer oligonucleotide, moreover it comprises a target sequence or equivalents thereof and a primer binding site for the first primer oligonucleotide. Further steps are explained in section “start nucleic acid chain”.

In a preferred embodiment, the second primer oligonucleotide at least in its 3′ segment comprises sequence portions that can complementary and sequence-specifically bind to a sequence segment of a target sequence and initiate/support a successful primer extension reaction by polymerase. The length of such a sequence segment comprises ranges of 6 and 40 nucleotides, better of 8 to 30 nucleotides, preferably of 10 to 25 nucleotides.

In one embodiment, the second primer oligonucleotide in its 3′ and 5′ segment comprises copyable sequence parts that are copied by polymerase in the synthesis of the first primer extension product. Thus, all sequence parts of the second primer are copied by polymerase. This leads to the formation of a primer binding site in the 3′ segment of the first primer extension product.

In one embodiment, the second primer oligonucleotide with its copyable portions in their lengths corresponds to the 3′ segment of the first primer extension product that is not bound by the activator oligonucleotide. In the complex comprising the second primer oligonucleotide and the first primer extension product the 3′ end of such a second primer oligonucleotide borders on the activator oligonucleotide that is bound to the first primer extension product. Extension of such a primer is done by using the first primer extension product as a template. In the extension of such a primer displacement of the activator oligonucleotide from the binding with the first primer extension product takes place by means of polymerase-dependent strand displacement. The corresponding second primer extension product is shown (primer extension product).

In a further embodiment, the second primer oligonucleotide with its copyable sequence portions is shorter than the 3′ segment of the first primer extension product that is not bound by the activator oligonucleotide. In the complex comprising the second primer oligonucleotide and the first primer extension product thus between the 3′ end of such a primer and the activator oligonucleotide bound to the first primer extension product there is a single-stranded section of the first primer extension product. Extension of such a primer is done by using the first primer extension product as a template. In the extension of such a primer displacement of the activator oligonucleotide from the binding with the first primer extension product takes place by means of polymerase-dependent strand displacement. The corresponding second primer extension product is shown in (primer extension product).

In a further embodiment, the second primer oligonucleotide with its copyable portions is longer than the 3′ segment of the first primer extension product that is not bound by the activator oligonucleotide. In the complex of the second primer oligonucleotide and the first primer extension product the 3′ segment of the second primer and the 5′ segment of the activator oligonucleotide compete for the binding to the first primer extension product. Binding of the 3′ segment of the second primer to the first primer extension product that is required for an initiation of the synthesis is with the simultaneous partial displacement of the 5′ segment of the activator oligonucleotide.

After initiation of the synthesis by polymerase there is the displacement of such a primer by using the first primer extension product as a template. In the extension of such a primer the displacement of the activator oligonucleotide from the binding with the first primer extension product is done by means of polymerase-dependent strand displacement. The corresponding second primer extension product is shown (primer extension product 6A, 6D, 6E). The sequence length of the 3′ segment of the second primer oligonucleotide that displaces the 5′ segment of the activator oligonucleotide can comprise the following regions: 1 to 50 nucleotides, better 3 to 30 nucleotides, preferably 5 to 20 nucleotides. Using second primer oligonucleotides of a greater length that exceeds the length of the 3′ segment of the first primer extension product is for example advantageous in some embodiments. Such embodiments comprise for example a first primer extension product with its 3′ segment that is not bound by the activator oligonucleotide being of a length of 5 to 40 nucleotides, better of 10 to 30 nucleotides. Especially with shorter 3′ segments a longer second primer oligonucleotide offers an improved sequence specificity in the initiation of synthesis.

The binding strength of the second primer oligonucleotide to its primer binding site depends on the length of the primer. Generally, longer second primer oligonucleotides can be employed with higher reaction temperatures.

Preferably, sequences of the first and second primer oligonucleotides and of the activator oligonucleotide are adapted to each other such that side reactions, e.g., primer dimer formation, are minimized. For that, for example the sequence of the first and second primer oligonucleotides are adapted to each other such that both primer oligonucleotides are not able to start an amplification reaction in the absence of an appropriate template and/or a target sequence and/or a start nucleic acid chain. This can be achieved for example in that the second primer oligonucleotide does not comprise a primer binding site for the first primer oligonucleotide and the first primer oligonucleotide does not comprise a primer binding site for the second primer oligonucleotide. Moreover, it is to be avoided that the primer sequences comprise extended self-complementary structures (self-complement).

The synthesis of the second primer extension product is a primer extension reaction and forms an individual step in the amplification. The reaction conditions during this step are accordingly adapted. Reaction temperature and reaction time are selected such that the reaction can successfully take place. The preferred temperature in this step depends on the polymerase used and the binding strength of the respective second primer oligonucleotide to its primer binding site and comprises for example ranges of 15° C. to 75° C., better of 20 to 65° C., preferably of 25° C. to 65° C. The concentration of the second primer oligonucleotide comprises ranges of 0.01 μmol/l to 50 μmol/l, better of 0.1 μmol/l to 20 μmol/l, preferably of 0.1 μmol/l to 10 μmol/l.

In one embodiment, all steps of the amplification proceed under stringent conditions that prevent or decelerate the formation of non-specific products/by-products. Such conditions are for example higher temperatures, for example above 50° C.

If more than one specific nucleic acid chain has to be amplified in one batch, so in one embodiment preferably sequence-specific primer oligonucleotides are used for the amplification of the respective target sequences.

In one embodiment, the synthesis of the first and second primer extension products proceeds at the same temperature. In a further embodiment, the synthesis of the first and second primer extension products proceeds at different temperatures. In a further embodiment, synthesis of the second primer extension product and strand displacement by the activator oligonucleotide proceed at the same temperature. In a further embodiment, synthesis of the second primer extension product and strand displacement by the activator oligonucleotide proceed at different temperatures.

In One Embodiment an Allele-Specific Second Primer is Used in Combination with an Allele-Specific Activator Oligonucleotide.

The second primer can bind to the corresponding complementary position of a start nucleic acid or a nucleic acid chain to be amplified. Preferably, a second primer comprises at least one sequence segment which preferably can specifically bind to an allele-specific sequence variant of the target nucleic acid under the reaction conditions used, wherein polymerase is able to extend the thus formed perfect-match complex, so that this results in a first primer extension product.

In one embodiment the position of an allele-specific sequence in the primer comprises the 3′ terminal nucleotide. In a further embodiment the position of an allele-specific sequence in the primer comprises the at least one of the positions −1 to −6 nucleotides in the 3′ terminal segment of the second primer. In a further embodiment the position of an allele-specific sequence in the primer comprises at least one of the positions of −6 to at least −15 in the 3′ terminal segment of the second primer.

Such a primer further comprises sequence segments which can complementary and uniformly bind to all of the allele variants of a target sequence. Thus, the second allele-specific primer comprises sequence segments that both are target sequence-specific and such that are allele-specific.

Preferably, a combination of an allele-specific primer and an allele-specific activator oligonucleotide is used, wherein the sequence complementary to the 3′ segment of the second primer can complementary bind to the activator oligonucleotide.

Individual allele-specific primers may be combined in one group which covers all the variants of a target sequence. Such a group of allele-specific primers comprises at least two different allele-specific primers, since a polymorphous locus in a given position in the target sequence comprises at least two sequence variants. The allele-specific primers are constructed such that under stringent reaction conditions they preferably can form a perfect-match bond with their respective specific template and thus, use this specific perfect-match template to form the respective primer extension products under the catalytic action of the polymerase. Preferably, 3′-terminal nucleotides and/or 3′-terminal segments of allele-specific primers may be used to discriminate variants of target sequences and in this way may be adapted in their sequence composition to the respective variants such that such primers with the respective variant form a perfect-match double strand under stringent conditions. Generally, such perfect-match double strands may be well recognized by a polymerase and under suitable reaction conditions primer extension takes place. Thus, if an allele-specific primer interacts with another variant of a target sequence a mismatch double strand is formed. Generally, such mismatches result in a delay of the extension by a polymerase or in a deceleration of the entire reaction. In one embodiment allele-specific primers in the 3′ segment can comprise at least one phosphorothioate bond which protects allele-specific primers against 3′-5′ nuclease decomposition by a polymerase.

Thus, several allele-specific primers comprise sequence segments which for one group of allele-specific primers are substantially identic or uniform, respectively, as well as sequence segments which in the primers of one group are different and characteristic for the respective sequence variant of a target sequence. By including uniform sequence segments such primers are able to hybridize to the respective target sequence under reaction conditions. By including characteristic sequence segments, a respective primer can specifically bind to a sequence variant of the target sequence to form a perfect-match double strand. Preferably, the primers are constructed such that under the reaction conditions used binding to a target sequence to form a perfect-match double strand is preferred and binding to a target sequence to form a mismatch double strand is less preferred.

In a further embodiment a target sequence-specific second primer (but no allele-specific second primer) is used in combination with an allele-specific activator oligonucleotide.

The second primer can bind to the corresponding complementary position of a start nucleic acid or a nucleic acid chain to be amplified. Preferably, a second primer comprises at least one sequence segment that under the reaction conditions used preferably can sequence-specifically bind to sequence segments of a target nucleic acid chain (comprising for example a start nucleic acid and/or the nucleic acid chain to be amplified), wherein said binding substantially takes place independently of potentially present sequence differences in the polymorphous locus, wherein polymerase is able to extend a thus formed perfect-match complex, so that this results in a first primer extension product.

Binding of such a primer substantially takes place in the sequence segment of the target nucleic acid which for at least two allele variants of said target nucleic acid chains is uniform. Preferably, primer binding takes place in the sequence segment of the target nucleic acid that is uniform for all of the allele variants of said target nucleic acid.

Here, binding takes place such that the polymorphous locus of the target sequence lies in the 3′ direction from the second primer, so that in a primer extension reaction said polymorphous locus is copied by polymerase. Thus, a resulting second primer extension product comprises a complementary sequence to the polymorphous locus. This sequence lies in the 3′ direction from the second primer.

Thus, such a primer comprises sequence segments that preferably can complementary and uniformly bind to all of the allele variants of a target sequence. In order that differentiation of allele variants can take place such a primer has to be combined with at least one allele-specific activator oligonucleotide. Thus, allele discrimination takes place by the action of the activator oligonucleotide. Positioning of the polymorphous locus in the 3′ direction from the primer causes its localization in the third and/or second region of the activator oligonucleotide.

Preferred Embodiments Comprising Primer Oligonucleotides Having Additional Sequence Segments:

The above-mentioned structures of the first primer and the second primer may be summarized as so-called “base primer structure” or “minimum primer structure”.

Such base structures of oligonucleotides having a primer function (e.g., first primer oligonucleotide, second primer oligonucleotide, optionally third primer oligonucleotide, optionally fourth primer oligonucleotide, etc.) comprise sequence segments that are of advantage for carrying out the amplification method, for example the first and second regions of the first primer.

Such a base structure of primers may be extended by further additional sequence segments. Such additional sequence segments comprise structures which themselves certainly are not needed for carrying out the amplification method, but may be useful for other tasks.

Such additional sequence segments optionally may be inserted into a primer and employed for further functions or reactions, respectively. In this way, the primer extension products synthesized by polymerase (e.g., starting from the first and/or second primers) can be connected to such sequence segments. In this way, an integration of such additional sequence segments and primer extension products to a molecular structure is achieved. Such an integration may be of advantage in certain embodiments. A number of applications for primer sequences having additional sequence segments is known to the skilled person.

Several functions are known to the skilled person which are supported by additional sequence segments of the primer.

Insertion of additional structures may be used for example as a means for mediating an intermolecular or intramolecular binding. Several examples of such structures are known to the skilled person. For example, probes may be designed in accordance with such principle of an intramolecular binding, e.g., in connection with scorpion primers. For example, further sequence segments may be used to bind further oligonucleotides. Here, a sequence-specific intermolecular binding may be formed by using stringent conditions. Such interactions may for example be used to bind amplification products to a solid phase via complementary binding to immobilized oligonucleotides.

A further example is insertion of so-called adaptor sequences and/or use of further sequence segments for unique coding or sequence-specific labelling primers and primer extension products starting therefrom (so-called primer barcoding). This is used for example for NGS library preparation (St∪hlberg et al Nucleic Acids Res. 2016 Jun. 20; 44(11): e105). In sequence analysis of primer extension products sequences may later be assigned by such a labelling.

Yet another example is represented by the use of further sequence segments for inserting specific sequences with binding of certain proteins, e.g. restriction endonucleases, etc.

Yet another example is represented by the use of further sequence segments for inserting spacer sequences which shall not bind a specific interaction partner, but mainly are used to increase the distance between adjacent sequences.

Such additional sequences may either be positioned on the copyable portion of the primer or attached to the non-copyable portion of the primer. Several factors are relevant in determining whether or not a sequence segment is copied. For example, positioning of the sequence segment in the respective oligonucleotide in the used nucleotide modifications (e.g. C3, HEG, 2′-Ome, etc.) may decide whether or not a sequence segment is used as a template during a process step.

In one embodiment an additional sequence segment is inserted into the copyable region of the primer, e.g. on the 5′ segment of the copyable portion of the second primer, so that for example in reading the primer sequence during a synthesis operation of target sequence also additional sequence segments are read by polymerase. The length of such an additional sequence segment includes ranges of 3 to 50 nucleotides. The composition of said sequence segments in this embodiment allows the synthesis by a polymerase, i.e. said sequence segment functions as a template for polymerase-dependent synthesis. In such a segment for example natural nucleotides are used, e.g. dA, dG, dC, dT.

In a further embodiment additional sequence segments may for example be positioned at the 5′ terminus of the primer which should not be copied in the synthesis of specific amplification fragments comprising a target sequence. This may for example be achieved by positioning one or more modifications or chemical groups, which hinder polymerase from synthesizing a complementary strand (e.g. HEG, C3, a segment comprising 4-10 nucleotides having 2′-Ome modifications, etc.). Such a modification may for example be positioned at the 5′ terminus of the copyable portion of the second primer and hinder continuation of the synthesis. For example, an HEG group may be inserted at the 5′ end of the copyable segment of the second primer, and subsequently an additional sequence segment.

Moreover, an additional sequence segment may be positioned at the 5′ terminus of the second region of the first primer. By such a localization of additional sequence segments synthesis of a complementary strand during a regular synthesis of specific amplification products comprising a target sequence is prevented.

The length of such an additional sequence segment comprises regions of 3 to 50 nucleotides. The base composition for example can comprise natural nucleobases (A, G, C, T, U, inosines) or modifications at different positions of nucleotides (e.g. at the bases, such as 2-amino-adenine, iso-guanine, iso-cytosine, 5-propargyl uridine, 5-propargyl cytosine, or at the sugar phosphate backbone, such as for example LNA, 2′-Ome, 2′-halogene, etc.). In a certain embodiment a first primer and additional sequence segments are combined in one oligonucleotide. In a further certain embodiment, a second primer and additional sequence segments are combined in one oligonucleotide.

Such additional sequence segments are designed in one oligonucleotide such that they preferably do not prevent the amplification method of target sequences. For example, this is achieved in that inhibiting interactions with the structures of the primers or activator that are essential for the method are avoided or reduced, respectively. In a certain embodiment additional structures can form complementary double strand segments with other primer regions under chosen reaction conditions. However, preferably such double-stranded segments do not prevent a specific amplification of a target sequence. In a further certain embodiment such additional sequence segments do not interact with or bind to the first or second primer regions of the first primer. In a further embodiment such additional sequence segments do not interact with the activator oligonucleotide. In a further embodiment such additional sequence segments do not interact with other primers in the reaction. In a further embodiment such additional sequence segments do not interact with P1.1-Ext or P2.1-Ext or other amplification fragments comprising a target sequence. In a further embodiment such additional sequence segments do not form double-stranded regions with the first or second regions of the first primer that are stable under reaction conditions and completely prevent the function of the first or second regions.

In a certain embodiment such additional sequence segments do not interact with or bind to the second primer. In a further embodiment such additional sequence segments especially do not interact with the 3′ segment of the second primer.

In one embodiment the first primer at its 5′ terminus of the second region comprises an additional sequence segment of the first primer (additive sequence variant P1). Said segment optionally comprises a sequence of 10-50 nucleotides that does not interfere with the amplification method of target sequences (e.g. does not form secondary structures with primers). Moreover, said segment optionally comprises a sequence of about 5 to 15 nucleotides of the copyable first region of the first primer. The additive sequence variant P1 comprises natural nucleotides as monomers (A, C, G, T) and may potentially function as a template for a polymerase.

In one embodiment the second primer at its 5′ terminus comprises an additional sequence segment of the second primer (additive sequence variant P2). Said segment optionally comprises a sequence of 10-50 nucleotides that does not interfere with the amplification method of target sequences (e.g. does not form secondary structures with primers). Moreover, said segment optionally comprises a sequence of about 5 to 15 nucleotides of the copyable region of the second primer. The additive sequence variant P2 comprises natural nucleotides as monomers (A, C, G, T) and may potentially function as a template for a polymerase.

It has been observed that such oligonucleotides comprising a first primer and additive sequence variant P1 or oligonucleotides comprising a second primer and additive sequence variant P2 to a smaller extend are susceptible to side reactions than oligonucleotides only comprising a first primer or oligonucleotides only comprising a second primer. In a certain embodiment, for example generation and/or amplification of unspecific primer dimer structures may be delayed. Thus, in such a side reaction optionally formation of by-products not comprising a target sequence may be reduced or delayed. In this way, for example premature consumption of primers may be reduced or delayed. For example, it is of advantage to employ such oligonucleotides if side reactions of primers-dimers comprising the first primer (PD P1) or primers-dimers comprising the second primer (PD P2) result in the premature consumption of primers in the reaction. The use of primers with such additional structures (first primer with additive sequence variant P1 and/or second primer with additive sequence variant P2) in certain embodiments is of advantage if in an amplification reaction in specific reactions are observed. Such side reactions may be favored by several factors, these are among others:
prolonged reaction times (e.g., reaction times range between 1 hr and 100 hrs)
higher concentrations of primers are used (e.g., concentrations range between 1 μmol/l and 1 mmol/l)
higher concentrations of polymerase are used (e.g., concentrations are in ranges above 10 units/10 μl)
multiplex reactions (e.g., amplification of more than 10 different target sequences in one reaction batch)
high concentrations of complex nucleic acid chains in the reaction batch (e.g., concentrations above 1 mg hgDNA in 50 μl)

here, single factors may favor side reactions alone or in combination with other factors.

In general, it is possible to act against side reactions (e.g., unspecific primer-dimer formation) by optimizing reaction components and/or reaction conditions, for example by reducing concentrations of individual components, shorter reaction times, sequence designing of primer sequences, choosing more stringent reaction conditions. The additional sequence segments (oligonucleotides comprising a first primer and additive sequence variant P1 or oligonucleotides comprising a second primer and additional sequence variant P2) given in an advantageous embodiment represent a further possibility to delay certain side reactions.

In examples there are shown primer oligonucleotides with additional sequence segments. In said examples additional sequence segments are used that do not participate in the specific amplification of a target sequence and contribute to the delay of side reactions. Thus, oligonucleotides comprising a first primer and additive sequence variant P1 and oligonucleotides comprising a second primer and additive sequence variant P2 are used.

A skilled person will appreciate that an oligonucleotide in addition to a primer structure that is of advantage for the specific amplification of a target sequence (said structure may also be referred to as “base primer structure” or “minimum primer structure”) can also comprise further additional sequence segments (e.g., additive sequence variant P1 or additive sequence variant P2). Such additional sequence segments may bring about a lot of different further advantages or useful properties or functions, respectively.

Exponential Vs. Linear Amplification

If both complementary strands (the first primer extension product and the second primer extension product, wherein both primer extension products can be templates for the syntheses of the complementary strands) are synthesized substantially in parallel to each other in the same batch an exponential propagation of both primer extension products can occur during such a reaction.

The primer extension products re-synthesized during a synthesis process close the respective complementary sequence parts to primers used, so that new primer binding sites are generated. In this way, re-synthesized strands themselves can function as templates in the subsequent synthesis processes.

If substantially only one primer extension product is synthesized as a result of cyclically repeated synthesis processes, so a linear amplification of said primer extension product occurs.

In an advantageous embodiment of the method both primers are employed substantially in equally high concentrations or in concentration ranges that are approximately equally high.

In a further advantageous embodiment of the method at least one of both primers is employed in a higher concentration than its partner primer. Here, the differences in concentrations may be in ranges that are between 1:2 to 1:50, advantageously between 1:2 to 1:10.

This can result in an asymmetric amplification reaction in which the concentration of a primer extension product is accordingly higher than that of the other strand.

The examples cited below are to be stated only to demonstrate the method and are not to be interpreted as being limiting.

The structures, sequences, and reaction conditions given in the examples are only to represent and illustrate the mode of function of the method and do not function as a limitation.

### EXAMPLES

Material and Methods:

Reagents were commercially purchased from the following suppliers:
unmodified and modified oligonucleotides (Eurofins MWG, Eurogentec, Biomers, Trilink Technologies, IBA Solutions for Life Sciences)
polymerases NEB (New England Biolabs)
dNTPs: Jena Bioscience
intercalating Eva green dye: Jena Bioscience
buffer substances and other chemicals: Sigma-Aldrich
plastic goods: Sarstedt

Solution 1 (Amplification Reaction Solution 1):
potassium glutamate, 50 mmol/l, pH 8.0
magnesium acetate, 10 mmol/l
dNTP (dATP, dCTTP, dGTP), 200 μmol/leach
polymerase (Bst 2.0 WarmStart, 120,000 U/ml NEB), 12 units/10 μl
Triton X-100, 0.1% (v/v)
EDTA, 0.1 mmol/l
TPAC (tetrapropylammonium chloride), 50 mmol/l, pH 8.0
Eva green dye (the dye was employed in accordance with the manufacturer's instructions in dilution of 1:50).

Solution 2 (Amplification Reaction Solution 2)
1×isothermal buffer (New England Biolabs); in a single concentration the buffer contains:
20 mM Tris-HCl
10 mM (NH₄)₂SO₄
50 mM KCl
2 mM MgSO₄
0.1% Tween® 20
pH 8.8@25° C.
dNTP (dATP, dCTP, dUTP, dGTP), 200 μmol/l each
Eva green dye (the dye was employed in accordance with the manufacturer's instructions in dilution of 1:50)

All concentrations are indications of the final concentrations in the reaction. Deviations from the standard reaction are indicated accordingly.

The melting temperature (Tm) of the participating components was determined upon concentration of 1 μmol/l of the respective components in solution 1. Deviating parameters are indicated respectively.

General Information on Reactions

Primer extension reactions and amplification were performed at reaction temperatures of 55° C. and 65° C. in a standard manner. Deviations are indicated. Both cyclic temperature changes between 55° C. and 65° C. and isothermal reaction conditions were used, as given in the examples.

The reaction was started by heating the reaction solutions to the reaction temperature since Bst 2.0 polymerase Warmstart at lower temperatures is mainly inhibited in its function by a temperature-sensitive oligonucleotide (according to the manufacturer's specifications). The polymerase becomes increasingly more active from a temperature of ca. 45° C., at a temperature of 65° C. no differences between polymerase Bst 2.0 and Bst 2.0 Warmstart could be observed. In order to prevent the extensive formation of by-products (e.g., primer dimer) during the preparation phase of a reaction polymerase Bst 2.0 Warmstart was used. Deviations are specifically indicated.

The reaction was stopped by heating the reaction solution to above 80° C., e.g., 10 min at 95° C. At this temperature polymerase Bst 2.0 is irreversibly denaturized and the result of synthesis reaction cannot be changed later.

The reactions were carried out in a thermostat having a fluorimeter. For that, a commercial Real-Time PCR apparatus was used, StepOne Plus (Applied Biosystems, Thermofischer). The reaction volume by default was 10 μl. Deviations are indicated.

Both end-point detection and kinetic observations have been made. In end-point detections the signal was recorded for example by a nucleic acid-bound dye, e.g., by TMR (tetramethyl rhodamine, also referred to as TAMRA) or by FAM (fluorescein). The wavelengths for exciting and measuring the fluorescence signals of FAM and TMR are stored as the factory settings in the StepOne Plus Real-Time PCR apparatus. Also, an intercalating dye (Eva green) was used in end-point measurements, e.g., in measuring the melting curve. Eva green is an intercalating dye and an analogue of the frequently employed SYBR green dye, however, with a slightly less inhibition of polymerases. The wavelengths for exciting and measuring the fluorescence signals of SYBR green and Eva green are identical and stored as the factory settings in the StepOne Plus Real-Time PCR apparatus. Fluorescence can continuously be detected by means of built-in detectors, i.e. “online” or “real-time”. Since the polymerase during its synthesis synthesizes a double strand this technique could be used for kinetic measurements (real-time monitoring) of the reaction. Due to a certain cross-talk between color channels in the StepOne Plus apparatus a partially increased basal signal intensity was observed in measurements in which e.g., TMR-labeled primers were used in concentrations of more than 1 μmol/l (e.g., 10 μmol/l). It was observed that the TMR signal in the SYBR green channel leads to increased basic values. These increased basic values were taken into account in calculations.

The kinetic observations of courses of reactions were routinely recorded by means of fluorescence signals of fluorescein (FAM-TAMRA Fret pair) or intercalating dyes (Eva green). Time-dependence of the signal course was detected (real-time signal detection in the StepOne plus PCR apparatus). An increase of the signal during a reaction compared to a control reaction was interpreted depending on the structure of the batch. For example, an increase in the signal using EVA green dye was interpreted as an indication of an increase in the amount of double-stranded nucleic acid chains during the reaction, and thus, judged to be the result of a synthesis by DNA polymerase.

In some reactions a melting curve determination was performed following the reaction. Such measurements allow to draw conclusions about the presence of double strands that for example can absorb intercalating dyes and this way significantly enhance signal intensity of dyes. With the rising temperature the proportion of double strands decreases and also the signal intensity decreases. The signal depends on the length of the nucleic acid chains and on the sequence composition. Said technique is well known to the skilled person.

When using melting curve analysis in context with reactions that contained significant proportions of modified nucleic acid chains (e.g., activator oligonucleotides or primers) it was found that the signal of the Eva green dye can behave different for example between the B form of the DNA and the A form of modified nucleic acid chains. For example, in the B form of the double-stranded nucleic acid chains (usually taken on for classical DNA sections) a higher signal intensity was observed than with double-stranded nucleic acid chains having the same sequence of nucleobases that can take on an A-form-like conformation (e.g., by several 2′-O-Me modifications of nucleotides). This observation was taken into account when intercalating dyes were employed.

As needed, the reaction was analyzed by means of capillary electrophoresis and the length of fragments formed was compared to a standard. In preparation for the capillary electrophoresis the reaction mixture was diluted in a buffer (Tris-HCl, 20 mmol/l, pH 8.0, and EDTA, 20 mmol/l, pH 8.0) such that the concentration of labeled nucleic acids was ca. 20 nmol/l. Capillary electrophoresis was performed at GATC-Biotech (Konstanz, Germany) as contractual service. In accordance with the specifications of the supplier the capillary electrophoresis was performed on an ABI 3730 Capillary Sequencer under standard conditions for Sanger sequencing using a POP7 gel matrix at ca. 50° C. and a constant voltage (ca. 10 kV). The conditions used resulted in the denaturation of double strands, so that in the capillary electrophoresis the single-stranded form of nucleic acid chains was separated. Electrophoresis is a standard technique in the genetic analysis. The automated capillary electrophoresis is employed routinely in Sanger sequencing to this day. The fluorescence signal is continuously recorded during the capillary electrophoresis (usually using virtual filters), so that an electrophoretogram is generated in which the signal intensity correlates to the duration of the electrophoresis. With shorter fragments, e.g., unused primers, there is observed an early signal peak, with extended fragments there is a temporal shift of the signals proportional to the length of the extended regions. Thanks to controls with known lengths the length of extended fragments can be measured. Said technique is known to a skilled person and is also employed by default in fragment length polymorphism.

### Example 1 (FIG. 52)

Use of Human Genomic DNA as a Source of a Target Sequence

In this example the use of humane genomic DNA (hgDNA) as a source of a target sequence is shown. As the target sequence a sequence segment of the factor-V Leiden gene (Homo sapiens coagulation factor V (F5), mRNA, here referred to as FVL-Gene) was chosen.

Target Sequence:

(SEQ ID NO: 1)
5′ GTAA GAGCAGATCC CTGGACAGGC AA GGAATACAGGTA - 3′

The binding sequence for the first primer oligonucleotide is underlined. The second primer oligonucleotide with its 3′ segment binds to the reverse complement of the double-underlined sequence.

The first primer, the second primer as well as the activator oligonucleotide were designed and synthesized for the FVL mutation variant of the gene.

The first primer oligonucleotide (SEQ ID NO:2):

P1F5-200-AE2053
5′ AACTCAGACAAGATGTGATTTTTTTACCTGTAT [CUCU
GAUGCUUC] 1TACCTGTATTCC 3′

The segment used as the primer in the reaction is underlined.

A=2′-deoxy-adenosine

C=2′-deoxy-cytosine

G=2′-deoxy-guanosine

T=2′-deoxy-thymidine (thymidine)

Said oligonucleotide comprises the following modifications:

1=C3 linker was used for termination of the synthesis of the second primer extension product.

The segment of the primer [CUCU GAUGCUUC] comprised 2′-O-Me modifications and was used as the second primer region for binding the first region of the activator oligonucleotide.

A=(2′-O-methyl-adenosine)

G=(2′-O-methyl-guanosine)

C=(2′-O-methyl-cytosine)

U=(2′-O-methyl-uridine)

Said primer oligonucleotide comprises the first region (positions 1-12 from the 3′ end), the second region (C3 linker as well as positions 13-24 from the 3′ end) as well as a segment having an additive sequence variant P1 (positions 25-57 from the 3′ end). The first region and the second region are required to perform a specific amplification and may be combined as “base structure of the first primer” or “minimum structure of the first primer”. The additive sequence variant P1 represents one example of additional sequence segments that can be integrated at the first primer oligonucleotide. Positions 1-12 are used as a template in the synthesis of the second primer extension product. C3 modification and the second region prevent a continuation of the synthesis at positions 25-57 during a synthesis of the second primer extension product.

Primer 2:

P2G3-5270-7063
(SEQ ID NO: 3)
5′ CTACAGAACTCAGACAAGATGTGAACTACAATGTT 6
GCTCATACTACAATGTCACTTACTGTAAGAGCAGA 3′

The segment used as the primer in the reaction is underlined.

Said oligonucleotide comprises the following modifications:

6=HEG linker

A=2′-deoxy-adenosine

C=2′-deoxy-cytosine

G=2′-deoxy-guanosine

T=2′-deoxy-thymidine (thymidine)

Said primer oligonucleotide comprises a copyable region and a non-copyable region. The copyable region comprises (positions 1-13 from the 3′ end that can complementary bind to a sequence of the FVL gene within hgDNA and positions 14-35 that certainly do not complementary bind to the sequence of the FVL gene, but can bind to the first primer extension product during the amplification). The copyable region may be summarized as a “base structure of the second primer” or “minimum structure of the second primer”.

The uncopyable region (positions 36-70 from the 3′ end that do not complementary bind to the sequence of the FVL gene) is separated from the copyable region by an HEG modification, what prevents continuation of the synthesis at positions 36-70 during a synthesis of the first primer extension product. The uncopyable region represents an example of an additive sequence variant P2 that can be integrated at the second primer oligonucleotide.

The following activator oligonucleotide (SEQ ID NO:4) was used:

AD-F5-1001-503
5′ [UAAUCUGUAA GAGCAGAUCC CUGGACAGGC AA GGAAUAC]
AGGTAGAAGCATC AGAG X 3′
A = 2′deoxy-adenosine
C = 2′-deoxy-cytosine
G = 2′-deoxy-guanosine
T = 2′-deoxy-thymidine (thymidine)

The 5′ segment of the oligonucleotide [UAAUCUGUAA GAGCAGAUCC CUGGACAGGC AA GGAAUAC] comprises 2′-O-Me-nucleotide modifications:

Modifications:

A=(2′-O-methyl-adenosine)

G=(2′-O-methyl-guanosine)

C=(2′-O-methyl-cytosine)

U=(2′-O-methyl-uridine)

X=3′-Phosphate group for blockage of a possible extension by polymerase.

The nucleotides and nucleotide modifications are mutually linked to phosphodiester bonds. The 3′ end of the activator oligonucleotide is blocked with a phosphate group to prevent a possible extension by the polymerase.

The first primer in its first region comprises a sequence that can specifically bind to the sequence of the factor-V Leiden gene within the genomic DNA, so that a synthesis by a polymerase can be started. The second region of the first primer comprises a sequence that does not specifically hybridize to the sequence of the FVL gene. Moreover, the first primer comprises a further sequence segment that links to the 5′ end of the second region (additive sequence variant P1). Said segment does not take part in the specific amplification of the factor-5 Leiden segment. The function of said segment is mainly seen in the delay of side reactions.

The second primer comprises a segment in its 3′ segment that can specifically bind to the genomic DNA, so that a synthesis by a polymerase can be started. The 5′ segment of the second primer comprises a sequence that specifically does not hybridize to the sequence of the FVL gene. During the backward synthesis said sequence segment may be copied. The second primer comprises a further sequence segment that can neither specifically hybridize with the activator oligonucleotide nor with the first primer, nor with the second primer. Said segment was localized at the 5′ end of the second primer and is separated from the 5′ end of the primer by an HEG linker (additive sequence variant P2). Said segment does not take part in the specific amplification. The function of said segment is mainly seen in the delay of side reactions.

The activator oligonucleotide was constructed such that a perfect match situation to the sequence of the factor-V Leiden mutation of the FVL gene results. The activator oligonucleotide comprises a first, second and third region.

As the genomic DNA the WHO standard for FVL mutation was used. Before using it in the reaction DNA was denatured by heating (5 min. at 95° C.) and thus, transferred from the double-stranded state to the single-stranded state. With the help of said single-stranded hgDNA first a start nucleic acid chain was prepared by a primer extension. Subsequently, an exponential amplification was performed starting from said start nucleic acid chain. Specificity of the amplification is demonstrated by means of melting curve analysis and Sanger sequencing with a sequencing primer.

All reactions were carried out in amplification solution 1.

The dNTPs used comprised: dATP, dGTP, dCTP, dUTP (instead of dTTP).

As the polymerase Bst 2.0 warm-start polymerase by NEB was used.

The start nucleic acid chain was prepared as follows: About 50000 haploid genomic equivalents (HGE), 150 ng hgDNA, were contacted with the second primer (0.5 μmol/l) and Bst-2.0 warm start polymerase (about 1 unit) as well as dNTPs (ca. 250 μmol/l) under hybridization conditions (amplification solution 1, temperature of about 60° C.) in 50 ml reaction volume and incubated for ca. 10 min. During this phase the second primer was extended with the genomic DNA being used as a template. This results in a primer extension product, which can be used as a start nucleic acid. Upon completion of this reaction the reaction mixture was heated to 95° C. for ca. 10 min to separate said start nucleic acid chain from the template. Said reaction mixture was frozen and used as needed as a source of the start nucleic acid chain.

The specific amplification of the target sequence of the FVL gene takes place using 5 μl of the reaction mixture with the start nucleic acid chain (corresponds to ca. 5000 HGE).

The other reaction components (first primer, second primer, activator oligonucleotide, Eva-Green dye, polymerase Bst.2.0 warm start, dNTPs) were added, so that a reaction end volume of ca. 10 μl resulted. The end concentrations of the components were: first primer: 5 μmol/l, second primer: 2 μmol/l, activator oligonucleotide: 1 μmol/l, Eva-Green dye (1:50), polymerase Bst.2.0 warm start (ca. 8 units), dNTPs: ca. 250 μmol/l.

No hgDNA was added to the control batch.

The reaction was carried out in a Step-One Plus apparatus (Thermofisher Scientific). The reaction temperature was initially changed by cyclic changes (30 cycles) between 65° C. (5 min, including the detection step) and 55° C. (1 min) and subsequently, held constant for 1 hr at 65° C. (detection step every 2 min). The course of the reaction was monitored by signal detection of the EvaGreen dye. On completion of the reaction the reaction mixture was first brought to 95° C. for 10 min and subsequently, a melting curve of the products formed was measured.

A schematic flow of the amplification is illustrated in FIG. 25 to FIG. 28.

First there was prepared a start nucleic acid chain (FIG. 25, steps A to D). Subsequently, an exponential amplification was carried out to extend the first primer and the second primer and the activator oligonucleotide (FIG. 25E to FIG. 28).

As a result of the amplification amplification fragments are accumulated. The result of the detection of the amplification can be seen in FIG. 52. As can be seen, after ca. 2 hrs of the reaction there is a noticeable increase in the fluorescence (FIG. 52 A). Subsequent melting curve analysis shows a specific melting curve with Tm of 84° C. (FIG. 52 B).

Sequence Control (FIG. 52C):

Sequence control was done by means of a sequencing primer:

SEQP1F5-35-x03
(SEQ ID NO: 5)
5′- AAG CTCGACAAAAGAAC TCAG AG TGTG CTCGAC AC
TACCTGTATTCC TTGCC 3′
A = 2′-deoxy-adenosine
C = 2′-deoxy-cytosine
G = 2′-deoxy-guanosine
T = 2′-deoxy-thymidine (thymidine)

For sequence control the reaction mixture (after having measured the melting curve) was diluted with water (from ca. 1:10 to ca. 1:100) and each of the aliquots obtained was mixed with a sequencing primer (added in a concentration of 2 μmol/l). Said mixture was shipped by a commercial sequencing supplier (GATC-Biotec) and sequenced by means of Sanger sequencing as a commissioned sequencing. The electropherograms obtained were examined for concordance with the FVL sequence gene. As a result of the reaction the sequence of the FVL gene was identified.

### Example 2

Selective Amplification Reaction of Sequence Variants of a Target Sequence.

In this example the effect of a sequence variation in the template on the amplification was investigated. When the first primer oligonucleotide is extended, a complementary strand is formed which has a complementary sequence to the template and thus, comprises said variations in the sequence. In this way, it is to be examined which effect such a mismatch between a thus formed first primer extension product and an activator oligonucleotide has on the amplification. The position of the mismatch lies in the 3′ direction from the first primer and thus, is not checked by the primer, but by the activator oligonucleotide.

Thus, discrimination between single sequence variants of the target sequence takes place by means of activator oligonucleotide using a uniform first and second primer.

The following templates was used: Template (SEQ ID NO 6) having a sequence composition resulting in a first primer extension product with a perfect match concordance with the activator oligonucleotide:

M2SF5-M001-200
(SEQ ID NO: 6)
5′ GCT CATA CTACAATGTCA CT TA CTGTAA GAGCAGATCC
CTGGACAGGC AA GGAATACAGGTA AAAAA 3′
A = 2′-deoxy adenosine
C = 2′-deoxy cytosine
G = 2′-deoxy guanosine
T = 2′-deoxy thymidine (thymidine)

The binding sequence for the first primer oligonucleotide is underlined. The second primer oligonucleotide binds to the reverse complement of the double-underlined sequence.

Template (SEQ ID NO:7) having a sequence composition leading to a first primer extension product forming a mismatch with the activator oligonucleotide in one single base position (printed in bold):

M2SF5-WT01-200
(SEQ ID NO: 7)
5′ GCT CATA CTACAATGTCA CT TA CTGTAA GAGCAGATCC
CTGGACAGGC GA GGAATACAGGTA AAAAA 3′
A = 2′-deoxy adenosine
C = 2′-deoxy cytosine
G = 2′-deoxy guanosine
T = 2′-deoxy thymidine (thymidine)

The binding sequence for the first primer oligonucleotide is underlined. The second primer oligonucleotide binds to the reverse complement of the double-underlined sequence.

The following primers were used:

The first primer oligonucleotide (SEQ ID NO:2):

P1F5-200-AE2053
5′ AACTCAGACAAGATGTGATTTTTTTACCTGTAT [CUCU
GAUGCUUC] 1TACCTGTATTCC 3′

The segment used as the primer in the reaction is underlined.

A=2′-deoxy-adenosine

C=2′-deoxy-cytosine

G=2′-deoxy-guanosine

T=2′-deoxy-thymidine (thymidine)

Said oligonucleotide comprises the following modifications: 1=C3 linker was used for termination of the synthesis of the second primer extension product.

The segment of the primer [CUCU GAUGCUUC] comprised 2′-O-Me modifications and was used as the second primer region for binding the first region of the activator oligonucleotide.

A=(2′-O-methyl-adenosine)

G=(2′-O-methyl-guanosine)

C=(2′-O-methyl-cytosine)

U=(2′-O-methyl-uridine)

Primer 2:

P2G3-5270-7063
(SEQ ID NO: 8)
5′ CTACAGAACTCAGACAAGATGTGAACTACAATGTT 6
GCTCATACTACAATGTCACTTACTGTAAGAGCAGA 3′

The segment used as the primer in the reaction is underlined.

Said oligonucleotide comprises the following modifications:

6=HEG linker

A=2′-deoxy-adenosine

C=2′-deoxy-cytosine

G=2′-deoxy-guanosine

T=2′-deoxy-thymidine (thymidine)

The following activator oligonucleotide (SEQ ID NO:4) was used:

AD-F5-1001-503
5′ [UAAUCUGUAA GAGCAGAUCC CUGGACAGGC AA GGAAUAC]
AGGTAGAAGCATC AGAG X 3′
A = 2′-deoxy adenosine
C = 2′-deoxy cytosine
G = 2′-deoxy guanosine
T = 2′-deoxy thymidine (thymidine)

The 5′ segment of the oligonucleotide [UAAUCUGUAA GAGCAGAUCC CUGGACAGGC AA GGAAUAC] comprised 2′-O-Me nucleotide modifications:

Modifications:

A=(2′-O-methyl-adenosine)

G=(2′-O-methyl-guanosine)

C=(2′-O-methyl-cytosine)

U=(2′-O-methyl-uridine)

X=3′-phosphate group for blockage of a possible extension by polymerase.

The nucleotides and nucleotide modifications mutually are linked with phosphodiester bonds. The 3′ end of the activator oligonucleotide is blocked with a phosphate group to prevent a possible extension by the polymerase.

Four batches were prepared:

Batch 1 as the start nucleic acid chain contains template M2SF5-M001-200 (Perfect Match Situation) in a concentration of 300 fmol/l (corresponds to ca. 2×10\hat{ }6 copies/batch).

Batch 2 as the start nucleic acid chain contains template M2SF5-M001-200 (Perfect Match Situation) in a concentration of 300 amol/l (corresponds to ca. 2×10\hat{ }3 copies/batch).

Batch 3 contains no template and thus, forms a control.

Batch 4 as the start nucleic acid chain contains template M2SF5-WT01-200 (single Mismatch Situation) in a concentration of 300 μmol/l (ca. 2×10\hat{ }9 copies/batch).

Primer 1 was employed with 5 μmol/l, the activator oligonucleotide with 2 μmol/l, and primer 2 with 1 μmol/l.

The further reaction conditions were: amplification solution 2.

To simulate the presence of genomic DNA in the assay 100 ng freshly denatured fish DNA (salmon DNA) were added per reaction.

The thermal reaction conditions were cyclic alternating temperature changes, wherein a 2 min time interval at 55° C. was followed by a 5 min time interval at 65° C. each. The amplification was monitored over 100 cycles. Detection was done at 65° C. for EvaGreen fluorescence signal.

Successful amplification could be observed by an increase in the EvaGreen fluorescence signal over time.

The temperature changes and real time monitoring were carried out with the StepOne Plus Real-Time PCR apparatus by Thermofisher.

FIG. 53A shows a typical course of the EvaGreen signal. On the Y axis the increase in the fluorescence signal (Delta Rn) is plotted and on the X axis the reaction time (as the cycle number). The arrows indicate individual reaction batches. Here, the marked positions belong to the following batches:

| | |
|---|---|
| Arrow 1: 300 fmol/l perfect match template | (ca. 2 × 10\hat{ }6 copies/batch) |
| Arrow 2: 300 amol/l perfect match template | (ca. 2 × 10\hat{ }3 copies/batch) |
| Arrow 3: no template | |
| Arrow 4: 300 pmol/l mismatch template | (ca. 2 × 10\hat{ }9 copies/batch). |

The increase of the fluorescence signal can be seen both with the perfect match and the mismatch variant of the template, wherein the signal of the mismatch variant (4) appears later despite a 100-fold excess. It can be seen that the mismatch amplification signals are significantly delayed over the perfect match amplification signal. With the single mismatch a delay of ca. 15 cycles is observed. The time lag (=cycle number) is a direct measure for the discrimination in the amplification. A further quantification of the discrimination in the amplification may be achieved by comparing it with a template concentration series under perfect match amplification.

When using a perfect match template, a complementary strand of a primer extension product is synthesized. Said extension product is complementary both to the perfect match template and to the activator oligonucleotide used.

In contrast, when using a mismatch sequence, a complementary strand of the extension product is generated in the synthesis of the first primer extension product, which certainly has a complete complementarity to the mismatch template, but that way deviates from the complementarity with the third region of the activator oligonucleotide. Said deviation takes place in the 5′-standing segment of the extension product which is to react with the activator oligonucleotide in order that the strand displacement process can proceed. As is shown in the preceding example, the mismatch interferes with a strand displacement by the activator oligonucleotide.

The control reactions with a perfect match template (arrows 1 and 2) showed a concentration dependency of the amplification. With decreasing concentration the reaction took more time to synthesize a sufficient amount of the nucleic acid to be amplified in order that the signal increases above the level of the baseline.

Said result illustrates the importance of the base composition in the activator oligonucleotide: Deviations from the complementarity between the activator oligonucleotide and the primer extension product may result in a deceleration or even interruption of the amplification.

In this example it has been shown that although sequence ends of a perfect match template and a mismatch template are consistent and thus, the potential to bind of both primer oligonucleotides was the same both reactions had a completely different course: in case of a complete complementarity between the activator oligonucleotide and the 5′ segment of the extension product of the first primer oligonucleotide amplification was as planned. An interruption of the strand displacement by a sequence deviation (in this case by a mismatch) resulted in the suppression of the amplification.

### Example 3

Demonstration of a Selective Amplification Reaction Using a Perfect-Match Primer and Different Activator Oligonucleotides: A Perfect-Match and a Mismatch Activator Oligonucleotide.

In this example we demonstrate how an allele-specific amplification reaction can be realized by a practical activator oligonucleotide design. For that, a target sequence after having added perfect-match and mismatch activator oligonucleotides, respectively, was amplified under cyclic temperature conditions. The detection of the corresponding amplification products in this example took place with the intercalating dye Eva Green.

The following template was used:
Template (SEQ ID NO:9) having a sequence composition leading to a first primer extension product with a perfect-match conformity with the activator oligonucleotide:

M2SF5-M001-200
(SEQ ID NO: 9)
5′ GCT CATA CTACAATGTCA CT TA CTGTAA GAGCAGATCC
CTGGACAGGC AA GGAATACAGGTA AAAAA 3′

The binding sequence for the first primer oligonucleotide is underlined.

The second primer oligonucleotide binds to the reverse complement of the double-underlined sequence.

The following primers were used:

The first primer oligonucleotide (SEQ ID NO:2):

P1F5-200-AE2053
5′ AACTCAGACAAGATGTGATTTTTTTACCTGTAT[CUCUGAUGCUUC]
1TACCTGTATTCC 3′

The segment used as a primer in the reaction is underlined.

A=2′-deoxy-adenosine

C=2′-deoxy-cytosine

G=2′-deoxy-guanosine

T=2′-deoxy-thymidine (thymidine)

This oligonucleotide comprises the following modifications:

1=C3 linker is used to terminate the synthesis of the second primer extension product. The segment of the primer [CUCU GAUGCUUC] comprised 2′-O-Me modifications and was used as a second primer region to bind the first region of the activator oligonucleotide:

A=(2′-O-methyl-adenosine)

G=(2′-O-methyl-guanosine)

C=(2′-O-methyl-cytosine)

U=(2′-O-methyl-uridine)
The second primer oligonucleotide (SEQ ID NO:10):

P2G3-5270-7063
(SEQ ID NO: 10)
5′ CTACAGAACTCAGACAAGATGTGAACTACAATGTT 6
GCTCATACTACAATGTCACTTACTGTAAGAGCAGA 3′

Modifications:

6=HEG linker

Both the first primer and the second primer are able to support an amplification with a perfect-match activator oligonucleotide.

The following perfect-match activator oligonucleotide (SEQ ID NO:4) was used:

AD-F5-1001-503
5′[UAAUCUGUAA GAGCAGAUCC CUGGACAGGC AA GGAAUAC]AGG
TAGAAGCATCAGAG X 3′
A = 2′-deoxy-adenosine
C = 2′-deoxy-cytosine
G = 2′-deoxy-guanosine
T = 2′-deoxy-thymidine (thymidine)

The 5′ segment of the oligonucleotide [UAAUCUGUAA GAGCAGAUCC CUGGACAGGC AA GGAAUAC] comprised 2′-O-Me nucleotide modifications:

Modifications:

A=(2′-O-methyl-adenosine)

G=(2′-O-methyl-guanosine)

C=(2′-O-methyl-cytosine)

U=(2′-O-methyl-uridine)

X=3′-phosphate group to block a possible extension by polymerase.

The following mismatch activator oligonucleotides (SEQ ID NO:11) were used:

MD2AD-F5-011-5
(SEQ ID NO: 11)
5′- GCTC TAATCTGTAA GAGCAGATCC CTG [GAC AGGC AA [symbol] G
AAUACAGG] TAGAAGCATC AGAG X 3′
A = 2′-deoxy-adenosine
C = 2′-deoxy-cytosine
G = 2′-deoxy-guanosine
T = 2′-deoxy-thymidine (thymidine)

The middle segment of the oligonucleotide [GAC AGGC AA [symbol] GAAUACAGG] comprised 2′-O-Me nucleotide modifications:

Modifications:

A=(2′-O-methyl-adenosine)

G=(2′-O-methyl-guanosine)

C=(2′-O-methyl-cytosine)

U=(2′-O-methyl-uridine)

X=3′-phosphate group to block a possible extension by polymerase.

The nucleotides and nucleotide modifications are mutually linked with phosphodiester bonds. The 3′ end is blocked with a phosphate group to prevent a possible extension by the polymerase.

In the mismatch activator oligonucleotide in position −24 (from the 3′ end) an A-nucleotide (mismatch variant) was employed instead of a G-nucleotide (perfect-match variant). The nucleotide is highlighted in bold. This position forms a mismatch with the 3′ terminal nucleotide of the first primer employed.

The template was employed in concentrations of 1-3 μmol/l. In the control reaction no template was employed (=negative control). Primer 1 was employed with 5 μmol/l, the respective activator oligonucleotide was employed with 2 μmol/l and primer 2 with 1 μmol/l.

The further reaction conditions were:
amplification solution 2
to simulate the presence of genomic DNA in the assay 100 ng of fresh denatured fish DNA (salmon DNA) were added per reaction.

The thermal reaction conditions were cyclic, alternating temperature conditions, wherein a 1 min time interval at 55° C. was followed by a 5 min time interval at 65° C. each. Amplification was typically monitored over 120 cycles, i.e. 120×(1 min 55° C.+5 min 65° C.)=120×6 min=12 h. Successful amplification could be observed by an increase in the EvaGreen fluorescence signal over time.

Analysis of the Allele-Specific Amplification Reactions

To analyze the amplification reaction and evaluate the amplification products formed the following technique was used:
Fluorescence signal of an intercalating dye (EvaGreen)
Melting curve analysis of the amplification products formed

FIG. 54 A shows a typical course of the EvaGreen fluorescence signal during an allele specific amplification reaction using a perfect-match activator oligonucleotide. The fluorescence signal of the EvaGreen dye is plotted on the Y axis and the reaction time (as cycle number) on the X axis. Arrow 1 in FIG. 54 designates a perfect-match amplification batch. Due to the perfect-match activator oligonucleotide the template can be amplified and from cycle 27 there is an increase in the fluorescence signal. Arrow 2 designates a negative control batch containing no template and thus, also does not generate an amplification signal during the experiment.

FIG. 54 B shows a typical course of the EvaGreen fluorescence signal during an allele specific amplification reaction using a mismatch activator oligonucleotide. The fluorescence signal of the EvaGreen dye is plotted on the Y axis and the reaction time (as cycle number) on the X axis. Arrow 1 designates a mismatch amplification batch. Due to the mismatch activator oligonucleotide the template cannot be amplified. Arrow 2 designates a negative control batch containing no template and thus, also does not generate an amplification signal during the experiment. The fluorescence signal of the mismatch activator oligonucleotide batch and negative control are almost identical, what shows how strong the amplification is suppressed in the mismatch situation.

FIG. 54 C shows the melting curve of the allele-specific amplification products of FIGS. 54A and 54B. The differentiation of the fluorescence signal against the temperature is plotted on the Y axis and on the X axis the temperature. Two characteristic peaks were found, designated with arrows 1 and 2. The peak in position 1 with a melting point close to 65° C. belongs to the mismatch activator oligonucleotide batch. The signal course cannot be distinguished from negative control batches (=amplification reaction without a template). This is to be contrasted with the peak in position 2 with a melting point close to about 85° C. Said peak belongs to the perfect-match activator oligonucleotide batch and is specific for the amplification product formed. The results suggest that specific amplification products having a defined melting point close to about 85° C. are formed in the perfect-match situation. On the other hand, with mismatch activator oligonucleotides the amplification reaction is suppressed. The fluorescence signals of the mismatch activator oligonucleotide batches in this example could not be distinguished from the negative control. This shows how strong the amplification is suppressed in the mismatch situation.

In summary, it is stated: a nucleotide mismatch in the activator oligonucleotide positioned in the second region of the activator oligonucleotide can prevent an amplification.

Based on this example it can be seen that sequence variant-specific amplification systems comprising a sequence variant-specific primer and a corresponding complementary sequence variant-specific activator oligonucleotide can be made up.

### Example 4

Amplification of Human FVL Target Sequence with Mutation in the Absence of Human Genomic DNA with Wild-Type Sequence Variant

The example shows a selective amplification of a target sequence comprising a polymorphous locus that comprises two sequence variants (mutation or wild-type) (SNP variants of Coagulation Factor V Leiden gene, FVL gene).

The components of the first amplification system (first primer, second primer, activator, polymerase) and reaction conditions were selected such that the nucleic acid to be amplified (amplification fragment 1.1 comprising both primer extension products, P1.1-Ext and P2.1-Ext) preferably comprised the sequence variant with mutation in the FVL gene. The wild-type sequence variant (WT sequence) should not significantly be amplified during the first amplification. The first amplification started starting from the start nucleic acid 1.1 that was obtained as a primer extension product using single-stranded human gDNA (template comprising a target sequence). The amplification was performed until the desired amount of amplification fragments was achieved. The products generated in the first amplification (amplification fragment 1.1) could be used as templates (start nucleic acid 2.1) for the second amplification (PCR).

The components of the second amplification system (third primer, fourth primer, polymerase) and reaction conditions (PCR amplification) were selected such that both variants of the target sequence could be amplified. The second amplification (PCR) took place subsequent to the first amplification in a separate step.

The products of the first amplification (amplification fragment 1.1) or the second amplification (amplification fragment 2.1) obtained were detected by means of different methods and analyzed. A detection method comprised real-time detection by means of intercalating dyes (Eva-Green dye), another detection method comprised real-time detection by means of sequence-specific oligonucleotide probes (Taqman probes) (said probes enable discrimination according to the sequence variant: mutation vs. wild-type variant), a further analysis method comprised Sanger sequencing of amplification fragments obtained in the amplification.

A demonstration of a sequence-specific amplification was achieved in several ways. On the one hand, amplification of a target sequence comprising a mutation variant could be achieved (either only by the first amplification or by a combination of the first and second amplifications). On the other hand, amplification of a target sequence comprising a mutation variant (100 or 10 copies) could be achieved in the presence of ca. 30000 copies of human gDNA with the wild-type variant. Further, it could be shown that no measurable amplification of the target sequence with the wild-type variant (5000 copies) took place under the selected conditions.

Materials and Methods:

Target Sequence:

As the target sequence having a polymorphous locus the following variants have been selected:

FVL Sequence Variant with Mutation:

(SEQ ID NO: 12)
5′- TGGGCTAATA GGACTACTTC TAATCTGTAA GAGCAGATCC
CTGGACAGGC AAGGAATACA GGTATTTTGT - 3′

Wild-Type Sequence Variant:

(SEQ ID NO: 13)
5′ - TGGGCTAATA GGACTACTTC TAATCTGTAA GAGCAGATCC
CTGGACAGGC GAGGAATACA GGTATTTTGT - 3′

Both sequences have been derived based on information available at NCBI:

Homo sapiens chromosome 1, GRCh38.p12 Primary Assembly, Sequence ID: NC_000001.11

Coagulation Factor V with SNP 1691G>A (substitution), in codon 506.

Components in the batch to prepare the start nucleic acid 1.1

Humane gDNA: WHO standard 04/224 for human gDNA for FVL gene
mutation-bearing variant of human gDNA (03/260 Factor V Leiden homozygote) (FVL mutation)
wild-type-bearing variant human gDNA (03/254 Wild Type Factor V) (WT sequence)
primer oligonucleotide

P1F5G2-2001-203
(SEQ ID NO: 14)
5′- CACAATCATCAATACTTTTTTGTCAAAATA[CUCU GAUGCUCU]
1GTCAAAATACCTGTATT
1 = C3

The underlined sequence consists of 2′-OMe modified nucleotides (2′-O-methyl nucleotides) and corresponds to the second region of the first primer.
Bst 2.0 WarmStart DNA Polymerase (NEB 120,000 units/ml) (in the reaction used in 1:1000 dilution), hereinafter referred to as “Bst polymerase”

First Amplification System
first primer oligonucleotide

P1F5G2-1001-103
(SEQ ID NO: 15)
5′-CACAATCATCAATACTTTTTTGTCAAAATA[CUCUGAUGCUCU]1GT
CAAAATACCT
1 = C3

The underlined sequence consists of 2′-OMe modified nucleotides and corresponds to the second region of the first primer.
Block oligonucleotide

B1-P1F5G2-3501-304
(SEQ ID NO: 16)
5′- [CUCUGAUGCUCUGUCAA]2AATACCTGAAA X
2 = HEG
X = 3′ phosphate group

The underlined sequence consists of 2′-OMe modified nucleotides.
Activator oligonucleotide

CF5G2-1002-401
(SEQ ID NO:17)
5′[AGGACUACUUCUAAUCUGUAAGAGCAGAUCCCUGGACAGGCAAGGAA
UACAGGUAUU]TTGACAGAGCATCAGAGAG X
X = 3′ phosphate group

The underlined sequence consists of 2′-OMe modified nucleotides.
Second primer oligonucleotide

P2-HAF5-081-1051
(SEQ ID NO: 18)
CACAATCATCAATACTAATAGGAC 6
CTCTGGGCTAATAGGACTACTTCTAATCTGTAAGAGCA
6 = HEG

Bst 2.0 WarmStart DNA Polymerase (NEB 120,000 units/ml), hereinafter referred to as “Bst polymerase”

The following concentrations for individual components were used:

| | |
|---|---|
| first primer oligonucleotide | 0.3 μmol/l |
| block oligonucleotide | 5 μmol/l |
| activator oligonucleotide | 2 μmol/l |
| second primer oligonucleotide | 0.2 μmol/l |
| Bst polymerase | was employed in the |
| | reaction in 1:1000 dilution. |

Human gDNA (by Promega, “Human male”) in the single-stranded form was added to the batches of the first amplification in concentration of ca. 100 ng per batch (12 μl). With that, a test system with a complex genetic background was simulated. The human gDNA comprises pooled samples (ca. 30000 copies per batch).

Second Amplification System (EvaGreen Detection)
third primer oligonucleotide

P3F5G2-1001-301
(SEQ ID NO: 19)
5′- CTCGACACTACTTCAAGGACAAAATACCTGTATTCC

(employed with 0.5 μmol/l)
fourth primer oligonucleotide

P4F5G2-1001-401
(SEQ ID NO: 20)
5′- CTC TGGGCTAATA GGACTACTTC TAATATGTAA GAGCA GAT

(employed with 0.5 μmol/l)
Taq polymerase Hot Start (NEB 5,000 units/ml) (employed in the reaction diluted 1:100)
detection by means of EvaGreen dye (Jena Biosciences Stock 1:50)

The third and fourth primers were designed in the “nested format” correspondingly relative to the first and second primers.

Oligonucleotide Probes to Detect FVL System in the Second Amplification:

Second Amplification System (Probe Detection)
third primer oligonucleotide

P3F5G3-1001-3303-4 (employed with 0.5 μmol/l)
(SEQ ID NO: 21)
5′- ACTTCAAGGACAAAATACCTGT ATT

fourth primer oligonucleotide

P4F5G3-1001-3404-2
(SEQ ID NO: 22)
5′- ATATGTAA GAGCA GAT CCC

(employed with 0.5 μmol/l)
Taq polymerase Hot Start (NEB 5,000 units/ml) (employed in the reaction diluted 1:100)
Detection by means of oligonucleotide probes

The third and fourth primers were designed in the “nested format” correspondingly relative to the first and second primers.
probe oligonucleotide for FVL mutation-specific detection

S1P4-FVL-1007
(SEQ ID NO: 23)
5′- FAM - TTGACAGGCAAGG AA 3′-MGB-NFQ

(by Thermofisher) (employed with 0.2 μmol/l)
probe oligonucleotide for WT-specific detection

S1P4-FVL-1003-1
(SEQ ID NO: 24)
5′- NED - TTCTGGACAGGCGAGG 3′-MGB-NFQ

(by Thermofisher) (employed with 0.6 μmol/l)
Discriminating Base is underlined.
NFQ=non fluorescent quencher (quencher of the BlackHole series)

Buffer:

All the reactions were carried out in the same buffer. 1×Buffer Components (NEB)

20 mM Tris-HCl

10 mM (NH₄)₂SO₄

50 mM KCl

2 mM MgSO₄

0.1% Tween® 20

pH 8.8@25° C.
optionally Eva-Green dye (Jena Biosciences) (employed in the reaction diluted 1:50).
dNTP mix for Bst polymerase: dATP, dCTP, dGTP 200 μmol/l each, dUTP 400 μmol/l
dNTP mix for Taq polymerase: dATP, dCTP, dGTP, dTTP 200 μmol/l each

Process Flow:

Preparation of a Start Nucleic Acid for the First Amplification

First, the start nucleic acid 1.1 comprising the target sequence was synthesized starting from the single-stranded human genomic DNA (WHO standards for FVL and WT sequence variants) using the primer (P1F5G2-2001-203, employed with 0.2 μmol/l) by primer extension with Bst polymerase (10 min at 50° C.) and subsequently detached from the template strand by thermal denaturation at 95° C. (5 min). Preparation of the start nucleic acid 1.1 for the sequence variant of the target sequence with FVL mutation and wild-type mutation was made in separate batches. Ca. 100000 copies of human gDNA have been employed per batch. The primer used for that was made up similar as the first primer oligonucleotide P1F5G2-1001-103.

Primer extension resulted in a start nucleic acid 1.1 which comprises a primer extension product having a sequence segment complementary to the target sequence and an overhang at the 5′ end. In the first amplification such a start nucleic acid can be used as a template: a second primer can sequence-specifically bind to such a start nucleic acid 1.1 and be extended by polymerase. An activator oligonucleotide can bind to the overhang with its first region.

Performing a first amplification and detection: The start nucleic acid 1.1 (with FVL mutation and/or WT sequence) was added to the amplification batch in different dilutions (100 copies, 10 copies for FVL mutation, and 5000 copies for WT sequence). The reaction was carried out under cyclic variations of the temperature between 50° C. and 65° C. There was no spontaneous decomposition of specific amplification fragments at these temperatures. One cycle comprised incubation at 50° C. (2 min) and at 65° C. (4 min). The batches were incubated for different times (up to ca. 15 hrs). The signal from the intercalating dye EvaGreen was detected during the course of the reaction. Melting curves were established after completion of the reaction. Amplification led to an increase in the amounts of double-stranded DNA (amplification fragments 1.1) which were able to intercalate intercalating dyes, what led to a signal increase of EvaGreen.

The amplification fragments 1.1 synthesized during said reaction could be used as templates for the second amplification or for Sanger sequence analysis, respectively.

Performing a Second Amplification and Detection (EvaGreen):

Before using them in the second amplification the products of the first amplification were diluted in a ratio of 1:6000. Amplification fragments present in these diluted batches were used as start nucleic acid 2.1 for PCR reaction. PCR reaction was performed in 40 cycles. One cycle comprised: 55° C. 1 min, 68° C. 3 min, 95° C. 20 sec. The detection was carried out with Eva-Green dye. By observing the signal increase (Ct value) a relative amount of templates added could be estimated at the beginning of the reaction. The amplification fragments 2.1 synthesized in the second amplification could be used for Sanger sequencing.

Performing a Second Amplification and Detection (Probe Oligonucleotides):

Before using them in the second amplification the products of the first amplification were diluted in a ratio of 1:6000. Amplification fragments present in these diluted batches were used as start nucleic acid 2.1 for PCR reaction. PCR reaction was performed in 40 cycles. One cycle comprised: 57° C. 1 min, 95° C. 20 sec. The detection was carried out with sequence-specific oligonucleotide probes (Taqman probes with fluorescence reporter and MGB by Thermofisher). By observing the signal increase (Ct value) both a relative amount of templates added could be estimated at the beginning of the reaction and the sequence composition in the relevant sequence part of the synthesized products.

Results and Evaluation:

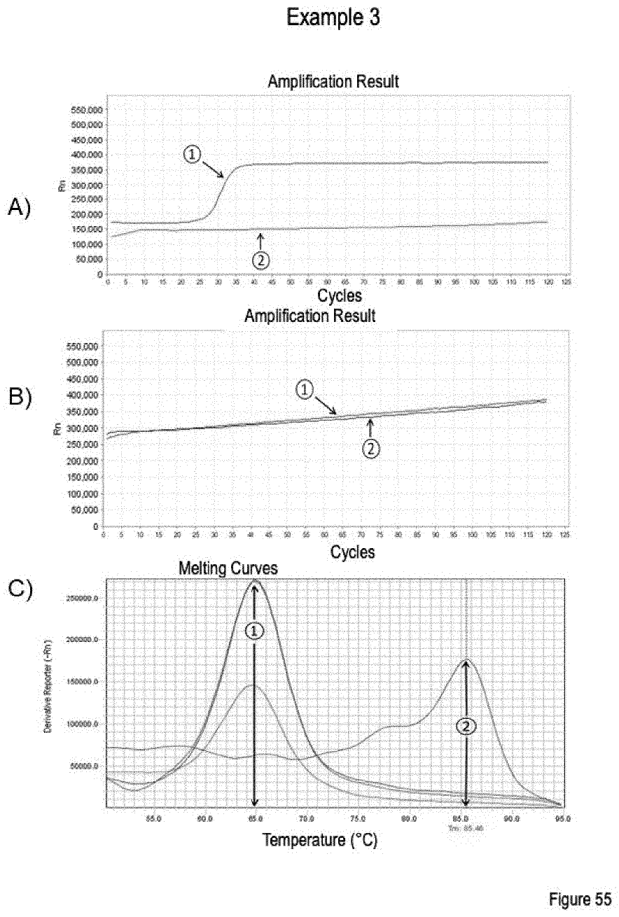

In the first amplification with batches starting from start nucleic acid 1.1 (target sequence with FVL mutation sequence variant) at initial concentrations of ca. 100 and 10 copies per batch (FIG. 55, arrows 1 and 2, A) signal course during amplification, B) melting curves) an increase in the signal (of EvaGreen dye) was detectable. In batches starting from start nucleic acid with target sequences of the WT variant at initial concentrations of ca. 5000 copies per batch no increase in the signal could be detected (FIG. 55, arrow 3). The signal was on the level of batches without a start nucleic acid. After completion of the first amplification an aliquot was taken, diluted, and added to a second amplification.

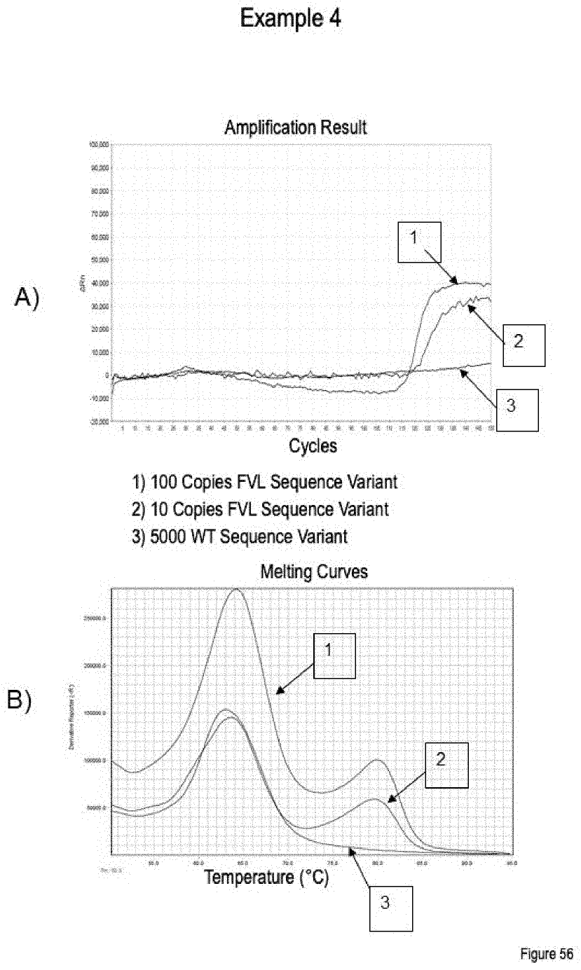

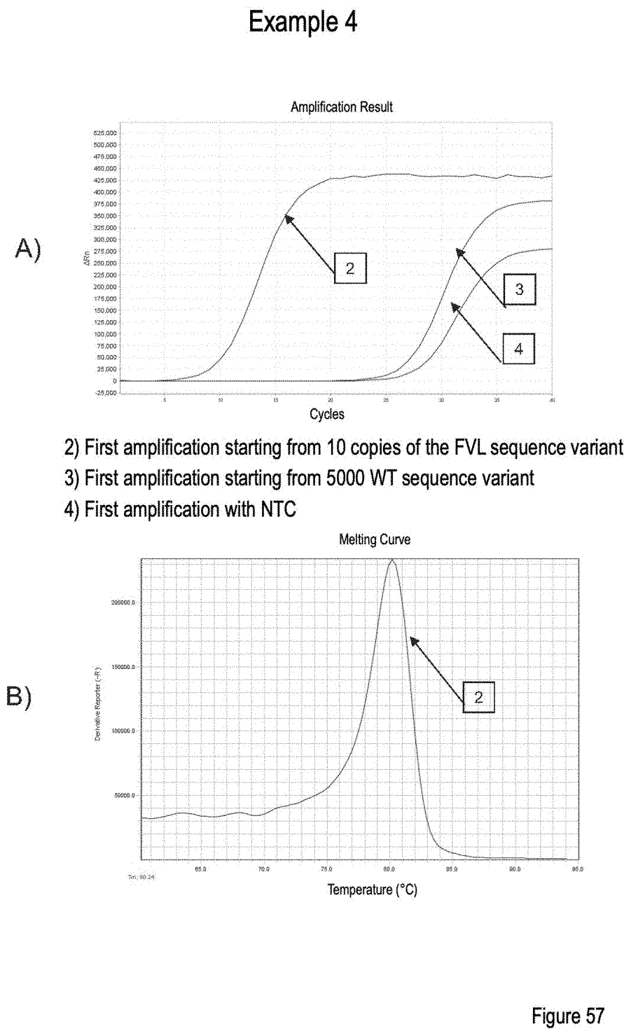

The second amplification was carried out starting from a dilution of batches of the completed first amplification (final dilution 1:6000). The second amplification used the third and fourth primers which were designed in the nested format to the first and second primers. The second amplification exhibited a specific increase in the signal in all batches of the first amplification with amplification fragments starting from start nucleic acid 1.1 with the target sequence of the FVL mutation variant (FIG. 56, arrow 2, A) shows signal course during amplification, B) shows melting curve). The increase in the signal takes place early during the PCR (Ct value ca. 5 or 8, respectively) and indicates a high concentration of amplification fragments of the first amplification. In batches starting from start nucleic acid 1.1 with the target sequence of the wild-type variant a signal was observed (FIG. 56, arrow 3, Ct value ca. 25) which was on the level of batches without start nucleic acid (FIG. 56, arrow 4, Ct value ca. 25). Comparison of time points of the increase in the signal (Ct values) with these batches shows that the amount of the amplification fragments with FVL mutation at the end of the first amplification was significantly higher than the amounts of amplification fragments with wild-type DNA. Taking into account the start nucleic acids employed (10 copies of FVL sequence variant vs. 5000 copies of the wild-type DNA) in the first amplification it could be observed that the amplification of the FVL sequence variant was significantly favored over the wild-type variant. The composition of the amplicons (FVL sequence) of single reactions was further confirmed by Sanger analysis and probe-based detection.

This result is indicative of a specific amplification of target sequences during the first amplification. The amplification fragments synthesized in the first amplification could be used as templates in the second amplification.

Distinction between two variants of the target sequence during the first amplification was by the effect of the activator oligonucleotide which was complementary designed to the target sequence having a mutation. Thus, said activator oligonucleotide comprises a mismatch to the wild-type variant of the target sequence in the nucleotide position. The activator oligonucleotide was designed such that the polymorphous locus of the target sequence lies in the third region of the activator oligonucleotide. Thus, the polymorphous locus was in the sequence segment of the target sequence which was in the 3′ direction from the first primer and in the 3′ direction from the second primer. The primers used alone could not have distinguished single sequence variants in the polymorphous locus of the target sequence.

During the first amplification there was mainly a selective multiplication of products which had sequences with a perfect complementary content to the third region of the activator (FVL target sequence having a mutation).

In this example an advantageous embodiment of the reaction conditions was selected in which on the one hand the primer concentration was smaller than the concentration of the activator oligonucleotide and on the other hand, cyclic temperature variations (between 50° C. and 65° C.) were used.

With the oligonucleotides used primer oligonucleotides form an interaction pair with the corresponding activator oligonucleotides (e.g., first primer oligonucleotide and first activator oligonucleotide). Said interaction pair under the reaction conditions of the amplification has a melting temperature of ca. 63° C. (measured at ca. 1 μmol/l concentration of both components under buffering conditions of the amplification). The first region of the primer oligonucleotide formed a complex having a melting temperature of ca. 50° C. with a complementary sequence portion of a template (e.g., first region of the first primer and the second primer extension product (P2.1-Ext), measured at a concentration of ca. 1 μmol/l of both components under the buffering conditions of the amplification).

Depending on the embodiment primers and activator oligonucleotides can be employed in different ratios.

In an advantageous embodiment, primer-activator combinations were used in which the concentration of primers was higher than the concentration of activator (e.g., primer 5 μmol/l and activator 2 μmol/l, see examples 1 to 3). In this embodiment there was an excess of primer, so that the primer extension step at a temperature of 50° C. proceeded with good yields, despite partial binding of the primer oligonucleotide to the activator oligonucleotide.

In a further advantageous embodiment (example 4) a mixture of a primer and a corresponding activator oligonucleotide was used in which the concentration of primer was lower than the concentration of the activator oligonucleotide (e.g., primer 0.5 μmol/l and activator 2 μmol/l). Thus, the activator oligonucleotide was in an excess. In this embodiment, reduction of the reaction temperature to 50° C. resulted in a rapid binding of the primer oligonucleotide to the activator oligonucleotide. This reduced the yield in the primer extension step at 50° C. and reduced the velocity of the amplification. In order to maintain a sufficient primer concentration also with low temperatures and thus, increase yields in the primer extension step, so-called block oligonucleotides have been employed. Such block oligonucleotides competed with the primer oligonucleotide for the bond to the activator oligonucleotide, but could not be extended by a polymerase themselves.

By using block oligonucleotides it was possible to employ the combination of primer oligonucleotide and activator oligonucleotide at some concentration ranges and to combine it with cyclic temperature variations. This resulted in an increase of the reaction velocity.

The structure of block oligonucleotides is substantially similar in design to the structure of primer oligonucleotides, with the following differences:
Block oligonucleotides are not extended by polymerase. This can be achieved for example by blocking the 3′ end by a modification (e.g., 3′ phosphate, 3′-C3, dideoxynucleotide), and/or by inserting terminal mismatches in the 3′ end of the block oligonucleotide.
Block oligonucleotides may vary in their sequence composition from their related primers. The number of sequence variations may be between 1 nucleotide and 20 nucleotides.

In designing block oligonucleotides, it is of advantage to keep the Tm of block oligonucleotides and activator oligonucleotides in a similar range, like the Tm of primer oligonucleotides and activator oligonucleotides. In this way, block oligonucleotides and primer oligonucleotide compete for the bond to activator oligonucleotide to a similar extent (e.g., Tm of primer-activator complex plus/minus 3° C.). By using higher concentrations of block oligonucleotides as primer oligonucleotides the binding ratio may suitably be affected.

Legend/Explanations from FIGS. 2, 18-21

FIG. 2 (Legend/Explanations)

S 1.1=first variant of the target sequence

S 1.2=second variant of the target sequence

N1=first uniform target sequence segment in which S1.1 is identical to S1.2

N2=polymorphous locus in which S1.1 is not identical to S1.2

N3=second uniform target sequence segment in which S1.1 is identical to S1.2

SN 1.1=first variant of a start nucleic acid to be expected

SN 1.2=second variant of a start nucleic acid to be expected

-----------------------

FIG. 18 (Legend/Explanations)

Variants of competitor primer P 5.1 for P1.1
P5.1 comprises no overhang for binding to activator oligonucleotide.
3′ end of P5.1 binds within the second blocking unit
P5.1 forms perfect match with SN 1.2 and may be extended by polymerase
P5.1 has the same length like the first region of P1.1

-----------------------

FIG. 19 (Legend/Explanations)

Variants of competitor primer P5.2 for P1.1
P5.2 comprises no overhang for binding to activator oligonucleotide.
3′ end of P5.2 binds within the second blocking unit
3′ terminal segment of P5.2 comprises at least one mismatch to SN 1.1
3′ terminal segment of P1.1 comprises at least one mismatch to SN 1.2
P5.2 forms perfect match with SN 1.2 and can be extended by polymerase
P5.2 has a longer binding segment on SN 1.2 than the first region of P1.1

-----------------------

FIG. 20 (Legend/Explanations)

Variants of competitor primer P 5.3 or P5.4 for P1.1
P5.3 comprises no overhang for the binding to activator oligonucleotide.
3′ end of P5.3 or P5.4 binds within the fourth blocking unit
P5.3 forms perfect match with SN 1.2 and can be extended by polymerase
P5.3 has a longer segment for binding to SN 1.2 than the first region of P1.1
P5.3 blocks the segment complementary to P1.1 in SN 1.2

-----------------------

FIG. 21 (Legend/Explanations)

Discrimination via activator oligonucleotide together with primer 2.1 with primer competition (competitor primer P6.1)

Variants of competitor primer P6.1 for P2.1,

The 3′-terminal segment of P6.1 forms perfect match with the complementary strand to SN 1.2

1. A method for amplification of a nucleic acid comprising the following steps:
a) hybridizing a first primer oligonucleotide to the 3′ segment of a strand of a nucleic acid chain to be amplified, wherein the nucleic acid chain to be amplified comprises a target sequence, wherein the first primer oligonucleotide comprises:
a first primer region in the 3′ segment of the first primer oligonucleotide that can sequence-specifically bind to a strand of a nucleic acid chain to be amplified,
a second region that is directly or via a linker linked to the 5′ end of the first primer region of the first primer oligonucleotide and that comprises a polynucleotide tail which is suitable for binding an activator oligonucleotide and supporting strand displacement (step c) by the activator oligonucleotide, wherein the polynucleotide tail remains substantially uncopied by polymerase under the selected reaction conditions,
b) extending the first primer oligonucleotide by means of a polymerase to form a first primer extension product comprising a sequence that is complementary to the target sequence of the nucleic acid chain (a) to be amplified,
c) binding the activator oligonucleotide to the polynucleotide tail of the second region of the first extended primer oligonucleotide, wherein the activator oligonucleotide comprises:
a first single-stranded region that can bind to the polynucleotide tail of the second region of the first primer oligonucleotide,
a second single-stranded region that is substantially complementary and can bind to the first region of the first primer oligonucleotide,
a third single-stranded region that is substantially complementary to at least a segment of the extension product, which has been synthesized by polymerase, of the first primer extension product,
wherein the activator oligonucleotide does not serve as template for a primer extension of the first primer oligonucleotide,
d) binding the activator oligonucleotide to the first primer region of the first extended primer oligonucleotide by displacing the strand of the nucleic acid chain to be amplified that is complementary to said first primer region,
e) binding the activator oligonucleotide to the complementary segment of the extension product of the first extended primer oligonucleotide by displacing the strand of the nucleic acid chain to be amplified that is complementary to said extension product, wherein the 3′ segment of the first primer extension product becomes single-stranded,
f) hybridizing a second oligonucleotide primer to the first primer extension product, wherein the 3′ segment of the second oligonucleotide primer comprises a sequence that can hybridize to the first primer extension product; and
g) extending the second oligonucleotide primer with polymerase to form a second primer extension product, wherein the extension takes place up to and including the first primer region of the first primer oligonucleotide and said first primer region is copied by the polymerase, wherein the polynucleotide tail of the second region remains uncopied,
h) repeating steps a)-g) until the desired degree of amplification has been achieved, characterized in that at least one of the components first primer, activator oligonucleotide, and second primer has a variation of one nucleotide to the perfect-match sequence of the target sequence, so that there is no perfect attachment of said component to the target sequence.

2. The method according to claim 1, wherein for at least one of the components first primer oligonucleotide, activator oligonucleotide, and second oligonucleotide primer, there are present such that are complementary to the target sequences and such that at least in one nucleotide differ from the complementary sequence of the target sequence, so that the detection of point mutations by amplification or no amplification can be detected.

3. The method according to claim 1, wherein at least three variants of at least one of the components first primer oligonucleotide, activator oligonucleotide, and second primer oligonucleotide are present, wherein each of these variants differs from the other variants by at least one nucleotide.

4. The method according to claim 1, wherein at least two variants of at least two of the components selected from first primer oligonucleotide, activator oligonucleotide, and second primer oligonucleotide are present, wherein each of these variants differs from the target sequence to which the variants are complementary to by at least one nucleotide.

5. The method according to claim 1, wherein the method is carried out substantially isothermal.

6. The method according to claim 1, wherein the third single-stranded region of the activator oligonucleotide is substantially complementary to the segment of the extension product, which has been synthesized by the polymerase, of the first primer extension product, which immediately follows the first primer region.

7. The method according to claim 1, wherein step (e) of the method is modified in that it comprises the binding of the activator oligonucleotide to the complementary segment of the extension product of the first extended primer oligonucleotide by displacing the strand of the nucleic acid chain to be amplified that is complementary to said extension product until said complementary strand of the nucleic acid to be amplified is detached from the first primer extension product, wherein the 3′ segment of the first primer extension product becomes single-stranded.

8. The method according to claim 1, wherein step (f) of the method is modified in that it comprises the hybridization of a second oligonucleotide primer to the first primer extension product, wherein at the same time there is at least a partial displacement of the activator oligonucleotide from the binding with the first extension product by strand displacement.

9. The method according to claim 1, wherein step (g) of the method is modified such that it comprises a displacement of the activator oligonucleotide from the binding with the first primer extension product with the participation of the polymerase.

10. The method according to claim 1, wherein step (h) of the method is modified in that it comprises the binding of the activator oligonucleotide to the uncopied polynucleotide tail of the first extended primer oligonucleotide and a displacement of the second primer extension product from the binding to the first primer extension product with the simultaneous formation of a complementary double strand with a segment of the first specific extension product of the first primer oligonucleotide.

11. The method according to claim 1, wherein the repetition of the steps is performed under such conditions that allow the repetition of the steps (a) to (g).

12. The method according to claim 1, wherein the method comprises the simultaneous amplification of the first and second primer extension products in an exponential reaction by using the first and second primer oligonucleotides and the activator oligonucleotide, wherein the formed primer extension products function as a template for the mutual synthesis.

13. Use of a kit for carrying out the method according to any of claim 1, wherein the kit contains at least one first primer oligonucleotide, at least one activator oligonucleotide, and at least one polymerase for amplification.

14. The use of a kit according to claim 13, wherein the kit further contains a second oligonucleotide primer.
